(19) United States
(12) Patent Application Publication
(10) Pub. No.: US 2021/0069371 A1
(43) Pub. Date: Mar. 11, 2021

(54) METHOD AND SYSTEM FOR DISPENSING A LIQUID FRAGRANCE INTO THE AIR

(71) Applicant: Savvy Inc., Toronto (CA)

(72) Inventors: Muneeb Muhammad MUSHTAQ, Mississauga (CA); Kashif Aftab AHMED, Scarborough (CA); Nabeel Muhammad MUSHTAQ, Mississauga (CA); John BYARD, Kleinberg (CA)

(21) Appl. No.: 17/014,257

(22) Filed: Sep. 8, 2020

Related U.S. Application Data
(60) Provisional application No. 62/897,055, filed on Sep. 6, 2019.

(51) Int. Cl. A61L 9/14; G01S 19/51; G01J 1/42

(52) U.S. Cl. CPC A61L 9/14; G01K 1/14; G01J 1/4204; G01S 19/51

(57) ABSTRACT

Systems and methods for dispensing a liquid fragrance into the air. The system includes a liquid fragrance dispenser. The liquid fragrance dispenser includes a dispenser controller for controlling operation of an atomizer. The dispenser controller, via the sensor subsystem, is configured to detect a value of an external variable of an environment of the liquid fragrance dispensing system. In response to detection of the value of the external variable, the dispenser controller variably controls an output of the liquid fragrance dispenser, for example, the duration of operation of the atomizer, the atomization output of the atomizer, or a user output interface. The system is configured to variably control the output of the dispenser, based on at least one or more of, a value of a detected external variable of the system, data representative of a liquid fragrance cartridge, and the previous atomization output of the atomizer, to achieve a target environmental fragrance condition.

### CROSS-REFERENCE TO RELATED APPLICATION(S)

None.

### FIELD

Example embodiments generally relate to systems and methods for controlling the dispensing of a fragrance into the air.

### BACKGROUND

A liquid fragrance may be dispensed into the air of a room to improve the ambiance and scent of the room. Some existing systems for dispensing a liquid fragrance may be limited to dispensing, heating, or aerosolizing the liquid fragrance at a constant rate of output once a manual switch or power source is turned on. This can lead to wastage of the liquid fragrance, as well as energy consumed, when there is more output than required. As well, the environmental conditions of the room can change over time and can vary from room to room.

Additional difficulties with existing systems may be appreciated in view of the Detailed Description of Example Embodiments, herein below.

### SUMMARY

An example embodiment is a liquid fragrance dispensing system, comprising a liquid fragrance dispenser. The liquid fragrance dispenser comprises an atomizer that is configured to atomize a liquid fragrance in fluid communication with the atomizer and output the atomized liquid fragrance; a dispenser memory; and a dispenser controller for controlling operation of the atomizer, and for executing instructions stored in the dispenser memory that, when executed, causes the dispenser controller to detect a value of an external variable of an environment of the liquid fragrance dispensing system, the external variable having a plurality of possible values, and in response, variably control an output of the liquid fragrance dispenser.

According to any one of the preceding example embodiments, the liquid fragrance dispensing system comprises a sensor subsystem that is configured to sense the external variable and provide data in relation to the sensed external variable, and the dispenser controller is configured to receive the data from the sensor subsystem for the detecting of the value of the external variable.

According to any one of the preceding example embodiments, the output of the liquid fragrance dispenser is the atomization output of the atomizer, wherein the dispenser controller is configured to: determine a target environmental fragrance condition information; determine an actual environmental condition based on the amount of atomization output of the atomizer and the value of the external variable detected by the sensor subsystem; wherein the variably control includes the dispenser controller being configured to variably control the atomization output of the atomizer to achieve the target environmental fragrance condition based on the actual environmental condition.

According to any one of the preceding example embodiments, the sensor subsystem includes a temperature sensor; the actual environmental condition includes temperature; and wherein the target environmental fragrance condition is achieved further based on the temperature.

According to any one of the preceding example embodiments, the sensor subsystem includes a humidity sensor; the actual environmental condition includes humidity; and wherein the target environmental fragrance condition is achieved further based on the humidity.

According to any one of the preceding example embodiments, the liquid fragrance dispensing system further comprises an accelerometer, and the dispenser controller is configured to determine acceleration threshold information; receive, from the accelerometer, data representative of the acceleration of the housing detected by the accelerometer; perform a comparison of the acceleration threshold information and the acceleration of the liquid fragrance dispenser; and deactivate or not activate the atomizer when the acceleration of the liquid fragrance dispenser exceeds the acceleration threshold information.

According to any one of the preceding example embodiments, the target environmental fragrance condition is achieved further based on previous atomization output of the atomizer.

According to any one of the preceding example embodiments, the liquid fragrance dispenser further comprises a user interface subsystem having a lighting subsystem, wherein the output of the liquid fragrance dispenser includes light output from the lighting subsystem, wherein the sensor subsystem includes an occupancy detection sensor, and wherein the dispenser controller is configured to: receive, from the occupancy detection sensor, data representative of an object's proximity detected by the occupancy detection sensor; and wherein the variably control includes the dispenser controller being configured to variable activate the light output of the lighting subsystem based on the object's proximity.

According to any one of the preceding example embodiments, the output of the liquid fragrance dispenser is the atomization output of the atomizer, wherein the dispenser controller is configured to: receive a control command in response to a determination of a proximity of a user equipment and the liquid fragrance dispenser based on GPS coordinates of the user equipment and GPS coordinates of the liquid fragrance dispenser; and wherein the variably control includes the dispenser controller being configured to variably control the atomization output of the atomizer based on the proximity.

According to any one of the preceding example embodiments, the liquid fragrance dispenser further comprises a user interface subsystem having a lighting subsystem, wherein the output of the liquid fragrance dispenser includes lighting intensity from the lighting subsystem, wherein the sensor subsystem includes an ambient light sensor, and the controller is configured to: receive, from the ambient light sensor, data representative of an intensity of the ambient light detected by the ambient light sensor; and wherein the variably control includes the dispenser controller being configured to variably control the lighting intensity of the lighting subsystem in general proportion to the intensity of the ambient light.

According to any one of the preceding example embodiments, said variably controlling is based on a characteristic of the liquid fragrance.

According to any one of the preceding example embodiments, the characteristic includes at least one of: a fluid type of the liquid fragrance, an odor intensity scale of the liquid fragrance, a fragrance decay rate of the liquid fragrance, a concentration of a solute in the liquid fragrance, or a viscosity of the liquid fragrance.

According to any one of the preceding example embodiments, wherein the liquid fragrance dispenser further comprises a wireless communication subsystem, the system further comprising a liquid fragrance cartridge that is releasably couplable to the atomizer, the liquid fragrance cartridge comprising a container that defines a cavity to contain the liquid fragrance; a port for providing fluid communication to the cavity of the container; a cartridge memory for storing information representative of the liquid fragrance cartridge; a wireless communication subsystem; and a cartridge controller for executing instructions stored in the cartridge memory that, when executed, causes the cartridge controller to wirelessly transmit data representative of the liquid fragrance cartridge to the liquid fragrance dispenser; wherein the dispenser controller is configured to receive the data representative of the liquid fragrance cartridge transmitted by the cartridge controller.

According to any one of the preceding example embodiments, the variably control is based on the data representative of the liquid fragrance cartridge.

According to any one of the preceding example embodiments, the data representative of the liquid fragrance cartridge received by the dispenser controller from the cartridge controller includes is a unique identification of the liquid fragrance cartridge, wherein the dispenser controller is configured to: validate the unique identification of the liquid fragrance cartridge from the dispenser memory or through communication with an external device.

According to any one of the preceding example embodiments, the dispenser controller is configured to: in response to successful validation from the validating, activate the atomizer.

According to any one of the preceding example embodiments, the liquid fragrance dispensing system further comprising the liquid fragrance, wherein: the liquid fragrance is contained in the cavity of the container; and the atomizer is disposed in fluid communication with the liquid fragrance.

According to any one of the preceding example embodiments, the transmitted data to the dispenser controller is representative of a concentration of a solute in the liquid fragrance cartridge and a viscosity of the liquid fragrance contained in the container of the liquid fragrance cartridge; and the dispenser controller is configured to: determine a target environmental fragrance condition; determine an actual environmental condition based on the atomization output of the atomizer, the concentration of the solute in the liquid fragrance, and the viscosity of the liquid fragrance; wherein the variably control includes the dispenser controller being configured to variably control the atomization output of the atomizer to achieve the target environmental fragrance condition based on the actual environmental condition.

According to any one of the preceding example embodiments, the dispenser controller is configured to: receive, from the sensor subsystem, the data in relation to the sensed external variable; wherein said determining of the actual environmental condition is further based on the data in relation to the sensed external variable.

According to any one of the preceding example embodiments, the target environmental fragrance condition is achieved further based on previous atomization output of the atomizer.

According to any one of the preceding example embodiments, the dispenser controller is configured to: transmit to the cartridge controller data representative of run time and atomization output of the atomizer; and the cartridge controller is configured to: receive from the dispenser controller the data representative of run time and atomization output of the atomizer, and update to the cartridge memory, based on the amount of the liquid fragrance in the cartridge, the run time, and the atomization output, an amount of fragrance used from the cartridge or a remaining amount of liquid fragrance in the cartridge.

According to any one of the preceding example embodiments, the cartridge controller is configured to: transmit, to the dispenser controller, data representative of an amount of liquid fragrance in the cartridge; the dispenser controller is configured to: convert data representative of run time and atomization output of the atomizer to data representative of an amount of the liquid fragrance used by the atomizer, transmit to the cartridge controller the data representative of the amount of liquid fragrance used by the atomizer or a remaining amount of liquid fragrance in the cartridge; and the cartridge controller is configured to update to the cartridge memory the amount of fragrance used from the cartridge or the remaining amount of liquid fragrance in the cartridge.

According to any one of the preceding example embodiments, the dispenser controller is configured to perform the receiving of the data representative of the unique identification of the liquid fragrance cartridge using a short range communication protocol.

According to any one of the preceding example embodiments, the short range communication protocol is a radio-frequency identification (RFID) protocol or a near field communication (NFC) protocol.

According to any one of the preceding example embodiments, the liquid fragrance dispenser comprises a housing that includes a body that defines a cavity for the releasably coupling of the liquid fragrance cartridge.

According to any one of the preceding example embodiments, the housing includes a cover that is couplable to the body for covering and uncovering the cavity, and the atomizer is housed in the cover.

According to any one of the preceding example embodiments, the cover holds the atomizer, wherein the cover is displaceable between a first position and a second position, wherein: in the first position, the atomizer is disposed relative to the cavity wherein, while the cartridge is received in the cavity, fluid communication between the liquid fragrance contained in the cartridge and the atomizer is absent; and in the second position, the atomizer is disposed relative to the cavity wherein, while the cartridge is received in the cavity, fluid communication between the liquid fragrance and the atomizer is present.

Another example embodiment is a liquid fragrance cartridge, comprising a container that defines a cavity to contain a liquid fragrance; a port for providing fluid communication to the cavity of the container; a memory for storing information representative of the liquid fragrance cartridge; a wireless communication subsystem; and a controller for executing instructions stored in the memory that, when executed, causes the controller to wirelessly transmit data representative of the liquid fragrance cartridge.

According to any one of the preceding example embodiments, the data representative of the liquid fragrance cartridge includes data representative of characteristics of the liquid fragrance contained in the container.

According to any one of the preceding example embodiments, the stored data representative of characteristics of the liquid fragrance includes information representative of a fluid type of the liquid fragrance contained in the container, and wherein the transmit data includes transmitting data representative of the fluid type.

According to any one of the preceding example embodiments, the stored data representative of characteristics of the liquid fragrance includes information representative of a rating on an odor intensity scale of the liquid fragrance contained in the container, and wherein the transmit data includes transmitting data representative of the rating on the odor intensity scale.

According to any one of the preceding example embodiments, the stored data representative of characteristics of the liquid fragrance includes information representative of a fragrance decay rate of the liquid fragrance contained in the container, and wherein the transmit data includes transmitting data representative of the fragrance decay rate.

According to any one of the preceding example embodiments, the stored data representative of characteristics of the liquid fragrance includes information representative of a concentration of a solute in the liquid fragrance contained in the container, and wherein the transmit data includes transmitting data representative of the concentration of the solute.

According to any one of the preceding example embodiments, the stored data representative of characteristics of the liquid fragrance includes information representative of a viscosity of the liquid fragrance contained in the container, and wherein the transmit data includes transmitting data representative of the viscosity.

According to any one of the preceding example embodiments, the memory stores information representative of a unique identification of the liquid fragrance cartridge, and wherein the transmit data includes transmitting data representative of the unique identification.

According to any one of the preceding example embodiments, the controller is configured to receive data representative of confirmation of validity of the unique identification of the liquid fragrance cartridge.

According to any one of the preceding example embodiments, the controller is configured to perform the transmitting of the data representative of the unique identification of the liquid fragrance cartridge using a short range communication protocol.

According to any one of the preceding example embodiments, the short range communication protocol is a radio-frequency identification (RFID) protocol or a near field communication (NFC) protocol.

According to any one of the preceding example embodiments, the memory stores information representative of an amount of liquid fragrance in the container, wherein the transmit data includes transmitting data representative of the amount of liquid fragrance in the container.

According to any one of the preceding example embodiments, the controller is configured to: receive data representative of run time and atomization output of an atomizer; and update in the memory, based on the run time and the atomization output, an amount of fragrance used from the container or a remaining amount of the liquid fragrance in the container.

According to any one of the preceding example embodiments, the controller is configured to: transmit data representative of an amount of liquid fragrance in the container; receive data representative of an amount of the liquid fragrance used by an atomizer or a remaining amount of the liquid fragrance in the container; and update in the memory the amount of fragrance used from the container or the remaining amount of the liquid fragrance in the container based on the received data representative of the amount of the liquid fragrance used.

According to any one of the preceding example embodiments, the controller is configured to detect a presence of a liquid fragrance dispenser, and, after the detecting of the presence, perform the transmit data representative of the cartridge to the liquid fragrance dispenser.

Another example embodiment is a method of dispensing liquid fragrance with a liquid fragrance dispenser, the method being executed by a controller of the liquid fragrance dispenser, the liquid fragrance dispenser having an atomizer that is configured to atomize a liquid fragrance in fluid communication with the atomizer and output the atomized liquid fragrance, the method comprising detecting a value of an external variable of an environment of the liquid fragrance dispensing system, the external variable having a plurality of possible values; and in response to the detecting, variably control an output of the liquid fragrance dispenser.

Another example embodiment is a non-transitory computer-readable medium containing instructions executable by a controller of a liquid fragrance dispenser, the liquid fragrance dispenser having an atomizer that is configured to atomize a liquid fragrance in fluid communication with the atomizer and output the atomized liquid fragrance, the method comprising instructions for detecting a value of an external variable of an environment of the liquid fragrance dispensing system, the external variable having a plurality of possible values; and instructions for, in response to the detecting, variably control an output of the liquid fragrance dispenser.

Other examples and aspects will be apparent from the disclosure and drawings provided herein.

### BRIEF DESCRIPTION OF DRAWINGS

For a more complete understanding of the present example embodiments, and the advantages thereof, reference is now made to the following description taken in conjunction with the accompanying drawings, in which:

FIG. 1 is a block diagram of a liquid fragrance dispensing system for dispensing a liquid fragrance into the air, in accordance with an example embodiment;

FIG. 2 is a perspective view of a liquid fragrance dispenser, in accordance with an example embodiment;

FIG. 3 is a perspective view of the liquid fragrance dispenser of FIG. 2;

FIG. 4 is a schematic of a lighting subsystem of the liquid fragrance dispenser of FIG. 2;

FIG. 5 is a perspective view of the liquid fragrance dispenser of FIG. 2 and a sleeve;

FIG. 6 is a perspective view of the sleeve of FIG. 5;

FIG. 7 is a perspective view of an atomizer subsystem of the liquid fragrance dispenser of FIG. 2;

FIG. 8 is a top view of the cover of the liquid fragrance dispenser of FIG. 2;

FIG. 9 is a front view of a circuit board of the liquid fragrance dispenser of FIG. 2;

FIG. 10 is a front view of a circuit board of the liquid fragrance dispenser of FIG. 2;

FIG. 11 is a block diagram of the liquid fragrance dispenser of FIG. 2;

FIG. 12 is a perspective view of the liquid fragrance dispenser of FIG. 2 and two liquid fragrance cartridges;

FIG. 13A is a perspective view of a liquid fragrance cartridge;

FIG. 13B is an exploded view of the liquid fragrance cartridge of FIG. 13A;

FIG. 14 is a block diagram of the liquid fragrance cartridge of FIG. 13A and FIG. 13B;

FIG. 15 is a flow chart depicting a method of loading a liquid fragrance cartridge into a liquid fragrance dispenser, in accordance with an example embodiment;

FIG. 16 is a flow chart depicting a method of initializing a liquid fragrance dispenser and a user equipment, in accordance with an example embodiment;

FIG. 17 is a flow chart depicting a method of managing use of a liquid fragrance cartridge, in accordance with an example embodiment;

FIG. 18 is a flow chart depicting a method of variably controlling an output of a liquid fragrance dispenser; and

FIG. 19 is a flow chart depicting a method of determining an amount of liquid fragrance in a liquid fragrance cartridge.

Similar reference numerals may have been used in different figures to denote similar components.

### DETAILED DESCRIPTION OF EXAMPLE EMBODIMENTS

Example embodiments relate to a liquid fragrance dispensing system. In some embodiments, the liquid fragrance dispensing system includes a liquid fragrance dispenser. The liquid fragrance dispenser includes a dispenser controller for controlling operation of an atomizer. The dispenser controller, via the sensor subsystem, is configured to detect a value of an external variable of an environment of the liquid fragrance dispensing system, the external variable having a plurality of possible non-zero values. In response to detection of the value of the external variable, the dispenser controller variably controls an output of the liquid fragrance dispenser, for example, the duration of operation of the atomizer (e.g. activating the atomizer for a period of time; activating the atomizer for a period of time, and then deactivating the atomizer for a period of time; activating the atomizer for a period of time, then deactivating the atomizer for a period of time, and then re-activating the atomizer for a period of time), the atomization output of the atomizer (e.g. increase, decrease, or maintain the vibration of the atomizer disc), or a user output interface.

In some embodiments, the liquid fragrance dispensing system includes a liquid fragrance cartridge. The liquid fragrance cartridge includes a cartridge controller that is in operable communication with a cartridge memory. The cartridge controller is configured to transmit data representative of the liquid fragrance cartridge, for example, to the dispenser controller. The dispenser controller is configured to receive the data representative of the liquid fragrance cartridge transmitted by the cartridge controller, and based on said data, variably control the output of the liquid fragrance dispenser.

In some examples, the liquid fragrance dispensing system is configured to variably control the output of the dispenser, based on at least one of, or more of, a value of a detected external variable of the liquid fragrance dispensing system, data representative of the liquid fragrance cartridge, and the previous atomization output of the atomizer, to achieve a target environmental fragrance condition.

The liquid fragrance dispensing system, in some embodiments, dispenses a liquid fragrance into the air of, for example, a room or a vehicle. In some embodiments, the liquid fragrance is dispensed by atomization of the liquid fragrance, such that droplets of the liquid fragrance are dispensed or sprayed into the room. In some embodiments, atomizing or atomization, include making an aerosol or a spray, mist, vapor, fog, cloud or another form of the liquid which appears to be atomized.

In some embodiments, the liquid fragrance is a solution or emulsion of a fragrant compound in a carrier. The fragrant compound is, in some embodiments, an essential oil or a blend of essential oils. The carrier is, in some embodiments, water, an alcohol such as ethanol, or a mixture of water and alcohol. In some embodiments, the liquid fragrance has one or more other additives, for example, a surfactant, a detergent, an emulsifier or a denaturing additive. In one example, the liquid comprises a mixture of ethanol, one or more essential oils, and, optionally, one or more additives. In some embodiments, the ethanol is present at 50% by volume or more or 60% by volume or more. In some embodiments, the essential oil or oils are present at a range between 1% and 20% by volume, for example, between 5% and 15% by volume. In some embodiments, the essential oil or oils are present at 5% or more by volume. In some embodiments, the essential oil or oils are present at 10% or more by volume. In some embodiments, the essential oil or oils are present at 15% or more by volume. In some embodiments, the essential oil or oils are present at 20% or more by volume. The additive, or additives, in some embodiments, are present at up to 10% by volume. In some embodiments, using a relatively large amount of ethanol, for example, 50% by volume or more or 60% by volume or more, with one or more essential oils, produces a low viscosity mixture which allows for a low power atomizer to be used, for example, 10 V at 2 A or less, 5 V at 1 A or less, or at 5 V at 2 A or less. In some embodiments, by using a relatively large amount of ethanol, an aerosol is created, or at least a fine suspension, wherein the essential oils dissipate into the air rather than settling as droplets near the atomizer.

In some embodiments, for example, the liquid fragrance dispensing system is configured to variably control the output of the dispenser based on a characteristic of the liquid fragrance. In some embodiments, for example, the characteristic of the liquid fragrance includes at least one of: a fluid type of the liquid fragrance, an odor intensity scale of the liquid fragrance, a fragrance decay rate (e.g. fragrance half life) of the liquid fragrance, a concentration of a solute in the liquid fragrance, a viscosity of the liquid fragrance, a date of manufacturing of the liquid fragrance, and an expiration date of the liquid fragrance. In some embodiments, for example, one or more of these characteristics of the liquid fragrance affects the strength, potency, or intensity of the environmental fragrance condition when the fragrance is dispersed into the air of the room.

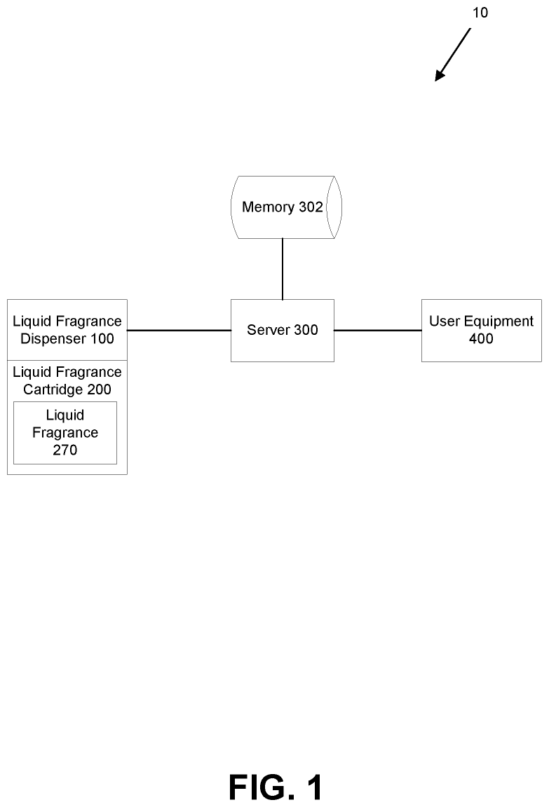

FIG. 1 illustrates a liquid fragrance dispensing system 10 for dispensing a liquid fragrance 270 into the air of the surrounding environment, in accordance with an example embodiment. The system 10 may include a liquid fragrance dispenser 100, a liquid fragrance cartridge 200 for containing the liquid fragrance 270, a server 300, and a user equipment 400. In some embodiments, the server 300 comprises at least one server and/or at least one cloud server.

In some embodiments, the liquid fragrance dispenser 100 is disposed in a premises, such as a dwelling, a building, and the like. Different liquid fragrance dispensers 100 may each be placed in a different premises, or in different areas of the same premises, such as different rooms of a building. The liquid fragrance dispenser 100 detects a value of at least one external variable of an environment in which the liquid fragrance dispenser 100 is placed.

In some embodiments, the server 300 is in a remote location from the premises in which the liquid fragrance dispenser 100 is placed. In some embodiments, the server 300 is a cloud-based server. The server 300 is configured to communicate with the liquid fragrance dispenser 100 and the user equipment 400 according to one or more communication protocols. In some embodiments, the server 300 communicates with the liquid fragrance dispenser 100 and the user equipment 400 in a secured manner, for example, via secured or encrypted communications. In some embodiments, the server 300 communicates with the liquid fragrance dispenser 100 and the user equipment 400 over the Internet (e.g. Wi-Fi) for example, or via short message service (SMS) in other examples, for notifying the liquid fragrance dispenser 100 to take certain action. In some embodiments, the server 300 provides a user interface, such as a web-portal, mobile application, or a dashboard for the user equipment 400 to connect to and control the liquid fragrance dispenser 100. In some embodiments, the server 300 includes a memory 302 for storing data from the liquid fragrance dispenser 100 and the liquid fragrance cartridge 200. The data can include a unique identifier of the liquid fragrance dispenser 100, and can include a unique identifier of the liquid fragrance cartridge 200. In some embodiments, the server 300 can communicate with the liquid fragrance cartridge 200 via the liquid fragrance dispenser 100. In some embodiments, a user can communicate with the liquid fragrance cartridge 200 via the user interface subsystem 155 of the liquid fragrance dispenser 100.

In some embodiments, the user equipment 400 is a computer, a laptop, smart phone, cell phone, a tablet, and the like. In some embodiments, the server 300 stores software updates to the liquid fragrance dispenser 100 and the liquid fragrance cartridge 200, and notifies the user equipment 400, for example, by using a flag to indicate that a software update is available. The user equipment 400 is configured to check the status of the software or the flag for software update in the server 300. In some embodiments, the server 300 notifies the user equipment 400 with the values of the detected external variables or data representative of the liquid fragrance cartridge 200 from the liquid fragrance dispenser 100 or from the liquid fragrance cartridge 200, for example, by a dedicated application, a web portal, emails, or short messages. In some embodiments, the user equipment 400 pushes the software from the server 300 to the liquid fragrance dispenser 100 or to the liquid fragrance cartridge 200 via a suitable communication modality over the Internet.

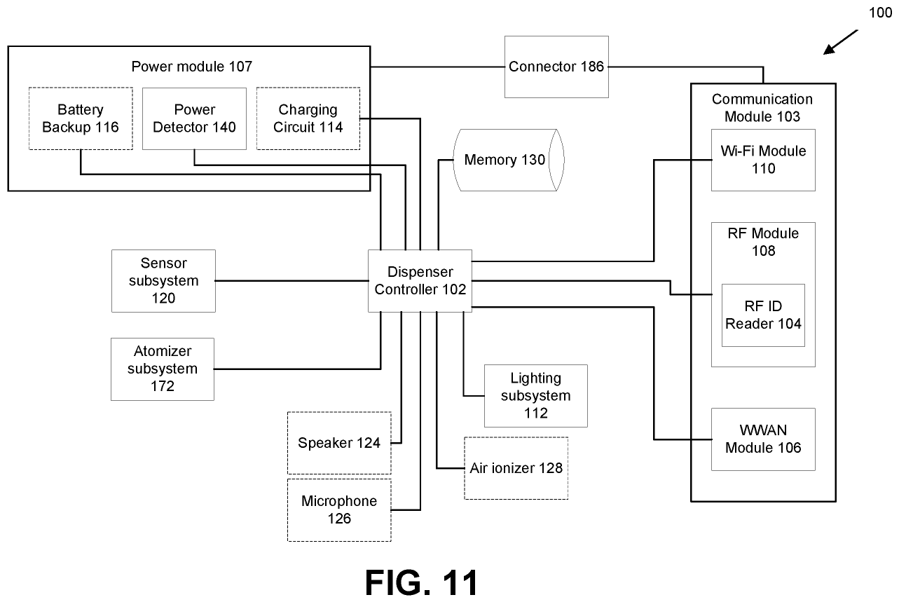

As depicted in FIG. 1, the liquid fragrance dispensing system 10 includes the liquid fragrance dispenser 100. The liquid fragrance dispenser 100 is placed in a suitable area of a premises, for detecting a value of an external variable of the environment of the liquid fragrance dispensing system 10, and, in response, variably control an output of the liquid fragrance dispenser 100. As depicted in FIG. 11, a dispenser controller 102 of the liquid fragrance dispenser 100 is in operable communication with an atomizer subsystem 172, a sensor subsystem 120, and a memory 130 of the liquid fragrance dispenser 100, for example, by sending commands to the atomizer subsystem 172, the sensor subsystem 120, and the memory 130, and receiving data from the atomizer subsystem 172, the sensor subsystem 120, and the memory 130. The dispenser controller 102 is for controlling operation of the atomizer subsystem 172. In some embodiments, the liquid fragrance dispensing system 10 includes a liquid fragrance cartridge 200 that can be removably inserted. In such embodiments, the liquid fragrance dispenser 100 is configured to be in operable communication with the liquid fragrance cartridge 200, via the dispenser controller 102, the dispenser communication module 103, a cartridge controller 202, and a cartridge communication module 203 of the liquid fragrance cartridge 200, for example, by sending data or commands to the liquid fragrance cartridge 200, or by receiving data or commands from the liquid fragrance cartridge 200. In some embodiments, the sensor subsystem 120 of the liquid fragrance dispenser 100 includes one or more sensors that are configured to detect or sense variables such as temperature and humidity of the external environment, the acceleration of the liquid fragrance dispenser 100, occupancy detection or proximity of nearby objects (such as a user or person), and the light intensity of the external environment.

In some embodiments, the liquid fragrance dispenser 100 is configured to communicate with the server 300, for example, by uploading the received sensor data of the values of the external variables or data representative of the liquid fragrance cartridge 200, as received from the sensor subsystem 120 and the liquid fragrance cartridge 200 respectively, to the server 300. The dispenser controller 102, via the sensor subsystem 120 of the liquid fragrance dispenser 100 and the cartridge controller 202, is configured to detect the value of one or more external variables or the data representative of the liquid fragrance cartridge 200, and send the associated data to the server 300. The server 300 is configured to store the uploaded data and notify a user equipment 400, for example, through an application, a web portal, by emails, or push notification. In some embodiments, data transmitted from the server 300 to the user equipment 400 is real-time ongoing data, for example, data representative of the actual environmental condition or the atomization output of the atomizer subsystem 172. In some embodiments, data transmitted from the server 300 to the user equipment 400 is specific notifications in response to determination by the dispenser controller 102 or the server 300 that a particular event occurred, based on the data representative of the value of one or more external variables or the data representative of the liquid fragrance cartridge 200. Example events include the liquid that is contained in the liquid fragrance cartridge 200 being below a threshold amount, or that the liquid fragrance dispenser 100 is tipped over.

The sensor subsystem 120 has one or more sensors that are each configured to sense or detect a variable that can be converted to analog or digital data. The sensor subsystem 102 is configured to sense the external variable and provide data in relation to the sensed external variable, and the dispenser controller 102 is configured to receive the data from the sensor subsystem 120 for the detecting of the value of the external variable. In some embodiments, the variable has a plurality of possible values. In some embodiments, the possible values of the variable includes a plurality of possible non-zero values. In some embodiments, the converted analog or digital data is in proportion to the variable being sensed. An example sensor is a temperature sensor. An example sensor is a humidity sensor. An example sensor is a sensor that is configured to detect the temperature of the liquid fragrance dispenser 100, such as the temperature of the sensor subsystem 120, the atomizer subsystem 172, the dispenser controller 102, or a user interface subsystem 155 of the liquid fragrance dispenser 100, such as a speaker 124, a microphone 126, or a lighting subsystem 112. An example sensor is an accelerometer that is configured to detect the acceleration of the liquid fragrance dispenser 100. An example sensor is an occupancy detection sensor that is configured to detect the distance, range, presence, or proximity of an object relative to the liquid fragrance dispenser 100. Example occupancy detection sensors include a time of flight sensor, a passive infrared detection sensor, and a microwave radar sensor. An example sensor is a light intensity sensor that is configured to detect the intensity of the ambient light.

In some embodiments, the sensor subsystem 120 includes one or more sensors that are wireless sensors that are configured for wireless communication. The sensor subsystem 120 wirelessly transmits data representative of the detected variable to the dispenser controller 102. In some embodiments, at least one of the sensors of the sensor subsystem 120 is disposed relative to the dispenser controller 102 such that the at least one sensor is within the range of a short range communication protocol of the liquid fragrance dispenser 100. In some embodiments, at least one of the sensors of the sensor subsystem 120 is placed out of range of a short range communication protocol of the liquid fragrance dispenser 100, but remains in operable communication with the liquid fragrance dispenser 100. In various examples, the wireless sensors include stand alone devices, wireless sensors that are part of the user equipment 400, or wireless sensors that are part of the liquid fragrance cartridge 200.

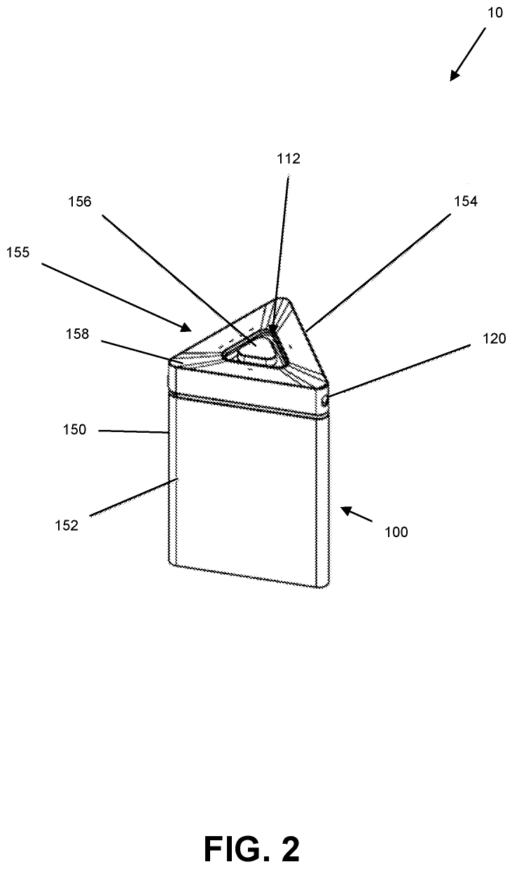

FIGS. 2 to 10 depict an example embodiment of the liquid fragrance dispenser 100. As depicted in FIG. 2, the liquid fragrance dispenser 100 includes a housing 150. In some embodiments, the housing includes a body 152 and a cover 154. As depicted in FIG. 2, in some embodiments, the liquid fragrance dispenser 100 has a shape similar to that of a triangular prism, with a longitudinal length and a triangular cross-sectional profile. In some embodiments, the liquid fragrance dispenser 100 has a cross-sectional profile other than a triangle, such as a circle, oval, square, rectangle, pentagon, hexagon, polygon, and the like. As depicted in FIG. 2, the corners of the cross-sectional profile of the liquid fragrance dispenser are rounded, to provide a desirable aesthetic and ease of holding, gripping, and manipulating the liquid fragrance dispenser 100.

In some embodiments, to provide electrical energy to the liquid fragrance dispenser 100, a power adapter is removably coupled to the liquid fragrance dispenser 100 and enables the liquid fragrance dispenser 100 to be in electrical communication with a standard 115 or 230 volt alternating current (AC) electrical outlet to power the fragrance dispenser 20. In some embodiments, the power adapter converts the AC power into low direct current (DC) power, e.g. 10 V or less or 5 V or less.

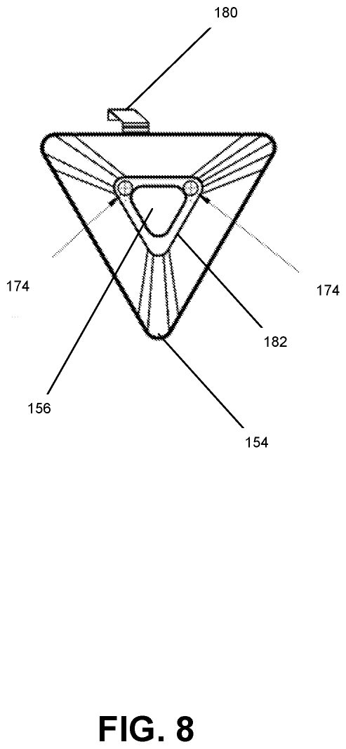

In some embodiments, electrical energy is provided to the liquid fragrance dispenser 100 via a universal serial bus (“USB”) connector 186, such as a USB Type C connector port. In some embodiments, the USB connector 186 and the dispenser controller 102 are mounted on a circuit board 184, as depicted in FIG. 8. In some embodiments, as depicted in FIG. 8, a portion of the USB connector 186 extends beyond the circuit board 184.

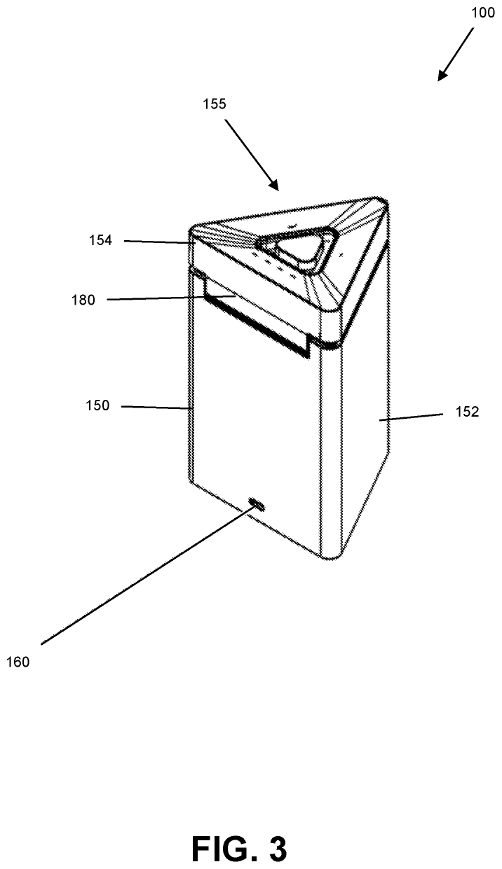

As depicted in FIG. 3, the body 152 defines a port 160 that is positioned to be aligned with the USB connector 186 when the liquid fragrance dispenser 100 is assembled. In some embodiments, where a portion of the USB connector 186 extends beyond the circuit board 184, the port 160 receives said portion of the USB connector 186. With the port 160 aligned with the USB connector 186, the corresponding USB connector may be connected to the USB connector 186 to provide electrical energy to the liquid fragrance dispenser 100.

In some embodiments, as depicted in FIG. 2 to FIG. 4, the liquid fragrance dispenser 100 includes a user interface subsystem 155, which includes a button 156, a capacitive touch surface 158, and a lighting subsystem 112.

In some embodiments, the button 156 is in operable communication with the dispenser controller 102, such that a user may input a command to the dispenser controller 102 via actuation of the button 156, for example, by applying a force to the button 156. In some embodiments, the button 156 is a spring-loaded button that will be depressed from an initial position upon actuation of the button 156, and return to the initial position upon release of said actuation. In some embodiments, the button 156 is a touch capacitive disc. In some embodiments, as depicted in FIG. 8, the button 156 is disposed in a channel 182 defined by the cover 154. In some embodiments, the button 156 is actuated to initialize the liquid fragrance dispenser 100. In some embodiments, the button 156 is actuated to change between modes of the liquid fragrance dispenser 100, for example, a continuous dispensing mode, a burst dispensing mode, a calibration mode, and the like. In some embodiments, the button 156 is actuated to activate the atomizer subsystem 172 to dispense a single burst of the liquid fragrance. In some embodiments, where the liquid fragrance dispensing system 10 comprises more than one liquid fragrance cartridge 200, the button 156 is actuated to activate the atomizer subsystem 172 to dispense a single burst of the liquid fragrances. In some embodiments, the button 156 is actuated to turn off the liquid fragrance dispenser 100.

In some embodiments, the capacitive touch surface 158 is in operable communication with the dispenser controller 102, such that a user may input a command to the dispenser controller 102 via actuation of the capacitive touch surface 158, for example, by touching the capacitive touch surface 158. In some embodiments, the capacitive touch surface 158 is a surface of the cover 154, for example, a top surface of the cover 154. In some embodiments, the capacitive touch surface 158 is actuated to activate the atomizer subsystem 172 to dispense a single burst of the liquid fragrance. In some embodiments, where the liquid fragrance dispensing system 10 comprises more than one liquid fragrance cartridge 200, for example, two liquid fragrance cartridges 200, the capacitive touch surface 158 is actuated, for example, by touching a first portion of the capacitive touch surface 158, to activate the atomizer subsystem 172 to dispense a single burst of one of the liquid fragrances, and capacitive touch surface 158 is actuated again, for example, by touching a second portion of the capacitive touch surface 158 that is different from the first portion, to activate the atomizer subsystem 172 to dispense a single burst of another one of the liquid fragrances.

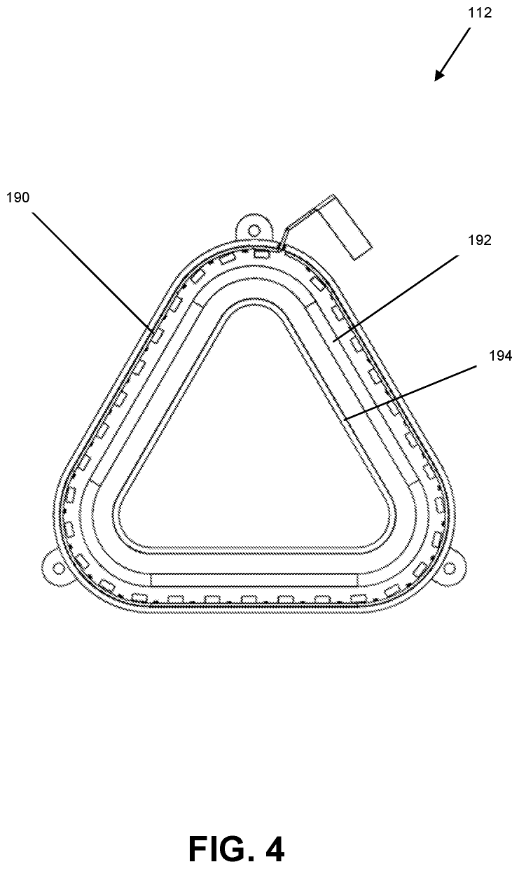

As depicted in FIG. 4, in some embodiments, the liquid fragrance dispenser 100 includes a lighting subsystem 112. In some embodiments, the lighting subsystem 112 includes a light 190, for example a light strip 190, and a light diffuser 192. In some embodiments, the light strip 190 includes a plurality of RGB LEDs. In some embodiments, the light diffuser 192 is a plastic light diffuser 192. In some embodiments, the lighting subsystem 112 is in operable communication with the dispenser controller 102, such that a control command may be transmitted to the lighting subsystem 112 for the lighting subsystem 112 to illuminate a certain colour or animation. In some embodiments, during initialization of the liquid fragrance dispenser 100, the dispenser controller 102 transmits a control command to the lighting subsystem 112 for the lighting subsystem 112 to illuminate a certain colour or animation, such a yellow colour. In some embodiments, during operation of the liquid fragrance dispenser 100, the dispenser controller 102 transmits a control command to the lighting subsystem 112 for the lighting subsystem 112 to illuminate a certain colour or animation, such as a green colour. In some embodiments, if the dispenser controller 102 determines that the amount of liquid fragrance in the liquid fragrance cartridge 200 is below a threshold amount, the dispenser controller 102 transmits a control command to the lighting subsystem 112 for the lighting subsystem 112 to illuminate a certain colour or animation, such as a red colour. Example animations include fading in, fading out, blinking, and the like.

In some embodiments, the user interface system 155 includes a microphone 126 to receive voice commands from a user. In some embodiments, the microphone 126 is in operable communication with an analog to digital converter, such that analog wave representative of a voice command are converted into digital data. In some embodiments, the digital data is filtered and segmented, and compared with a library of data representative of known words and phrases, for converting the voice command into a digital command that is transmittable to the dispenser controller 102 and processable by the dispenser controller 102.

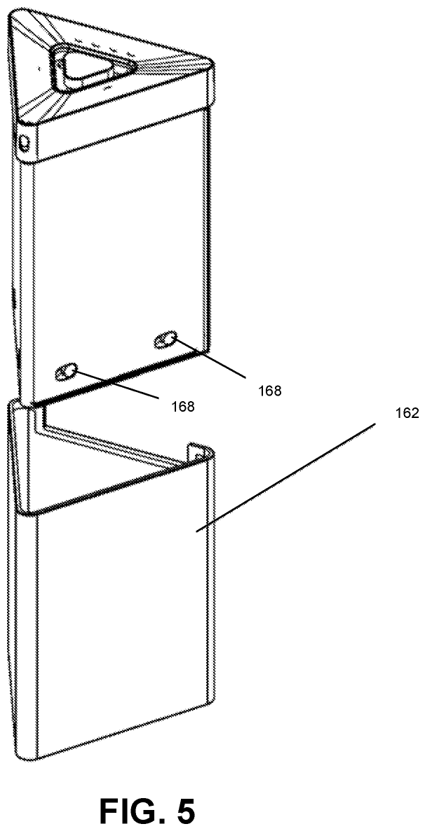

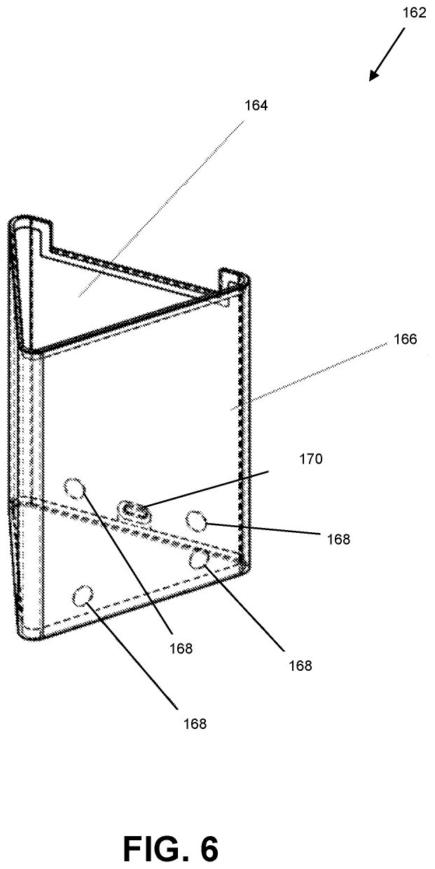

As depicted in FIG. 5 and FIG. 6, in some embodiments, the liquid fragrance dispensing system 10 includes a sleeve 162. The sleeve 162 is configured such that the body 152 is receivable in the sleeve 162. As depicted in FIG. 6, the sleeve 162 includes a shell 164 and a cloth 166. The shell 164 is configured to provide rigidity to the sleeve 162, while the cloth 166 is configured to provide a desirable texture or visual aesthetic to the sleeve 162. In some embodiments, the shell 164 and the cloth 166 are coupled, for example, using an adhesive.

The sleeve 162 is configured to be releasably coupled with the housing 150 of the liquid fragrance dispenser 100. As depicted in FIG. 6, in some embodiments, the sleeve 162 includes one or more magnets 168 for releasably coupling with corresponding magnets 168 of the housing 150. While the sleeve 162 is releasably coupled to the housing 150 and the housing 150 is received in the sleeve 162, the sleeve 162 provides a desirable visual aesthetic to the liquid fragrance dispenser 100.

As depicted in FIG. 6, in some embodiments, the sleeve 162 includes a port 170. While the sleeve 162 and the housing 150 are releasably coupled, the port 170 of the sleeve 162 is aligned with the port 160 of the housing 150, such that the USB connector 186 may be connected with a power source to provide electrical energy to the liquid fragrance dispenser 100.

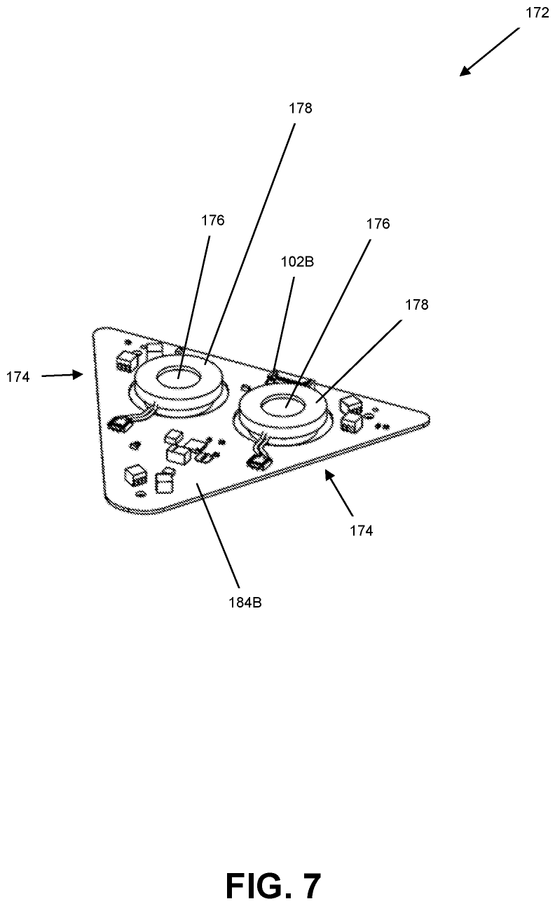

As depicted in FIG. 7, the liquid fragrance dispenser 100 includes an atomizer subsystem 172. The atomizer subsystem 172 includes including an atomizer 174. The atomizer subsystem 172 is configured to atomize a liquid fragrance in fluid communication with the atomizer 174 and output the atomized liquid fragrance. In some embodiments, the atomizer 174 includes an ultrasonic vibrating plate or a jet nebulizers. In some embodiments, the atomizer 174 is a piezoelectric atomizer or piezoelectric oscillator. In some embodiments, the atomizer 174 is a microporous atomizer, such as a 20 millimeter ceramic atomizer. The atomizer 174 is configured to generate a mist from a liquid in fluid communication with an atomizer disc 176 of the atomizer 174, via ultrasonic vibration of the atomizer disc 176, to break the surface tension of the liquid in fluid communication with the atomizer disc 176. In some embodiments, the atomizer disc 176 vibrates at a micrometer level to atomize the liquid. In some embodiments, the liquid is the liquid fragrance. A wicking pad 178 is disposed on top of the atomizer disc 176 to prevent liquid from pooling on the surface of the atomizer disc 176.

In some embodiments, the atomizer subsystem 172 and the dispenser controller 102 are in operable communication, such that the atomizer subsystem 172 may send data representative of its operational state, for example, whether the atomizer 174 is operational (e.g. operational state) or the frequency of vibration of the atomizer disc 176, and that the dispenser controller 102 may send a control command to the atomizer subsystem 172 to activate the atomizer subsystem 172, deactivate the atomizer subsystem 172, variably control the duration of operation of the atomizer subsystem 172 at an atomization output, for example, the amount of time the atomizer subsystem 172 is operational before the atomizer subsystem 172 is deactivated or the amount of time the atomizer subsystem 172 is operational before the atomizer subsystem 172 is deactivated and then reactivated, or variably control the frequency of vibration of the atomizer disc 176, thereby variably controlling the atomization output of the atomizer subsystem 172. When the frequency of vibration of the atomizer disc 176 is variably controlled to be larger or smaller, there is a corresponding change that occurs on the atomization output of the atomizer subsystem 172.

In some embodiments, the atomizer 174 is connected to or mounted on a circuit board 184B, as depicted in FIG. 7.

In some embodiments, the atomizer subsystem 172 includes more than one atomizer 174, for example, when the liquid cartridge dispenser 100 is configured to dispense a liquid from more than one liquid fragrance cartridge 200. In such embodiments, each liquid fragrance cartridge 200 has a corresponding atomizer 174. For example, where the liquid cartridge dispenser 100 is configured to dispense a liquid from a first liquid fragrance cartridge 200 and a second liquid fragrance cartridge 200, the atomizer subsystem 172 has two atomizers 174, as depicted in FIG. 7. In some embodiments, the dispenser controller 102 is configured to independent control the one or more atomizers 174 of the atomizer subsystem 172, in order to, for example, mix the dispensing of the liquids.

In some embodiments, the atomizer subsystem 172 is disposed in the cover 154, as depicted in FIG. 8. In some embodiments, the atomizer 174 is disposed in the cover 154, as depicted in FIG. 8. In such embodiments, at least a portion of the atomizer 174 is aligned with at least a portion of the channel 182, such that liquid that is atomized by the atomizer 174 may be dispersed through the channel 182. In some embodiments, as depicted in FIG. 8, while the liquid fragrance dispenser 100 is assembled, the atomizer 174 is disposed in the cover 154 and relative to the button 156 such that the liquid that is atomized by the atomizer 174 may be dispersed through the channel 182 between the cover 154 and the button 156.

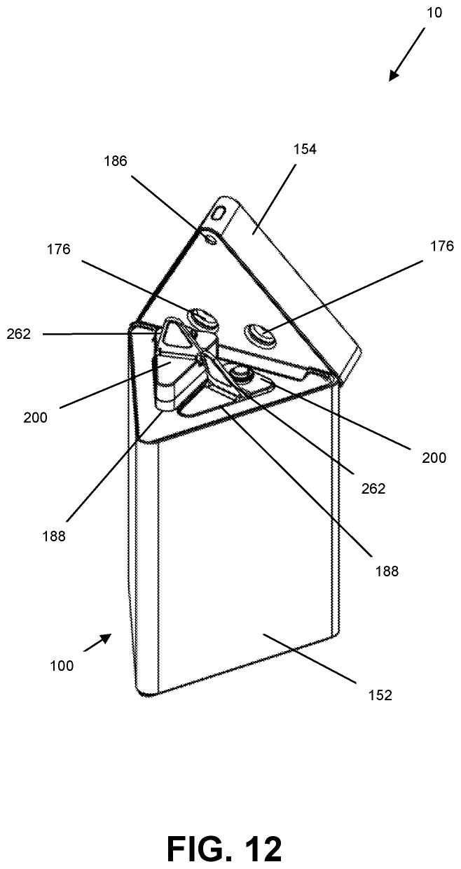

As depicted in FIG. 3 and FIG. 8, in some embodiments, the cover 154 is pivotably coupled to the body 152 by a hinge 180. In some embodiments, for example, the cover 154 is couplable to the body 152 for covering and uncovering the cavity 188. In such embodiments, the cover 154 is displaceable between a first position or an open position, as depicted in FIG. 12, and a second position or a closed position, as depicted in FIG. 2 or FIG. 3.

In some embodiments, the atomizer subsystem 172 is displaceable between a first position and a second position. In the first position, the atomizer subsystem 172 is disposed such that fluid communication between the atomizer 174 and liquid to be atomized by the liquid fragrance dispenser 100 is absent. In the second position, the atomizer subsystem 172 is disposed such that fluid communication between the atomizer 174 and liquid to be atomized by the liquid fragrance dispenser 100 is absent.

In some embodiments, where the atomizer subsystem 172 or the atomizer 174 is disposed in the cover 154, for example, housed in the cover 154, the atomizer subsystem 172 is disposed in its first position when the cover 154 is disposed in its first position, and the atomizer subsystem 172 is disposed in its second position when the cover 154 is disposed in its second position.

In some embodiments, while the liquid fragrance dispenser 100 is in an operable orientation, as depicted in FIG. 2 and FIG. 3, the atomizer subsystem 172 is disposed in an upper portion of the liquid fragrance dispenser 100, such that the atomized liquid is emitted in an upward direction out of the liquid fragrance dispenser 100. In some embodiments, the atomizer subsystem 172 is disposed above the liquid fragrance cartridge 200, such that the liquid fragrance is drawn in an upward direction towards the atomizer subsystem 172 to be atomized.

In some embodiments, the atomizer subsystem 172 runs on up to 80 V of electricity. In some embodiments, the liquid fragrance dispenser 100 includes a charge pump, and electrically converts 5 V of electricity, for example, from a power source, to up to 80 V of electricity to power the atomizer subsystem 172.

In some embodiments, the atomizer subsystem 172 does not require heating, fans, or propellants for causing the atomization of the liquid fragrance.

In some embodiments, the liquid fragrance dispenser 100 includes a dispenser controller 102. The dispenser controller 102 is in operable communication with the atomizer subsystem 172 and the dispenser memory 130. The dispenser controller 102 is for controlling operation of the atomizer subsystem 172, and for executing instructions stored in the dispenser memory 130 that, when executed, causes the dispenser controller 120 to detect a value of an external variable of an environment of the liquid fragrance dispensing system 10, the external variable having a plurality of possible values, for example, non-zero values, and in response, variably control an output of the dispenser 100.

In some embodiments, the dispenser memory 130 is rewritable memory.

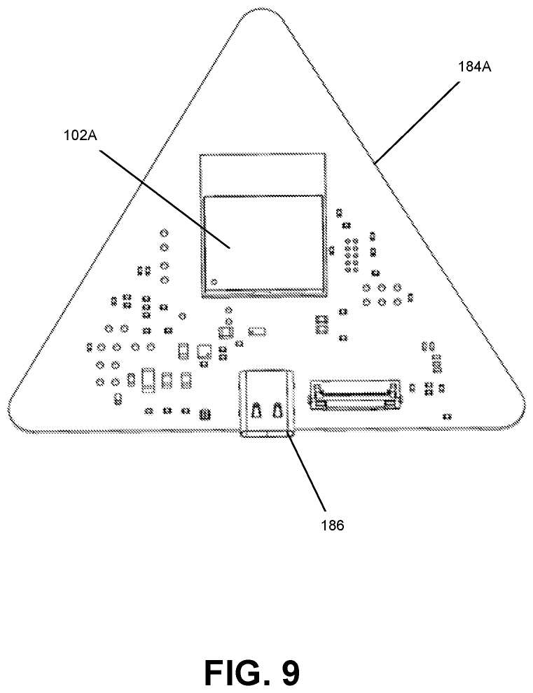

In some embodiments, the dispenser controller 102 is mounted on a circuit board 184A as depicted in FIG. 9. In some embodiments, the dispenser controller 102 is disposed in the body 152 of the housing 150 of the liquid fragrance dispenser 100. In some embodiments, the dispenser controller 102 is disposed at a bottom end of the body 152 of the housing 150 of the liquid fragrance dispenser 100. In some embodiments, the dispenser controller 102 controls the operation of the liquid fragrance dispenser 100. In some embodiments, the power management circuitry, a communication module 103 for wireless communication, and the memory 130 are mounted or printed on the circuit board 184A. The communication module 103 can be configured for communication via a number of suitable standards, such as Bluetooth, Wi-Fi, cellular, etc.

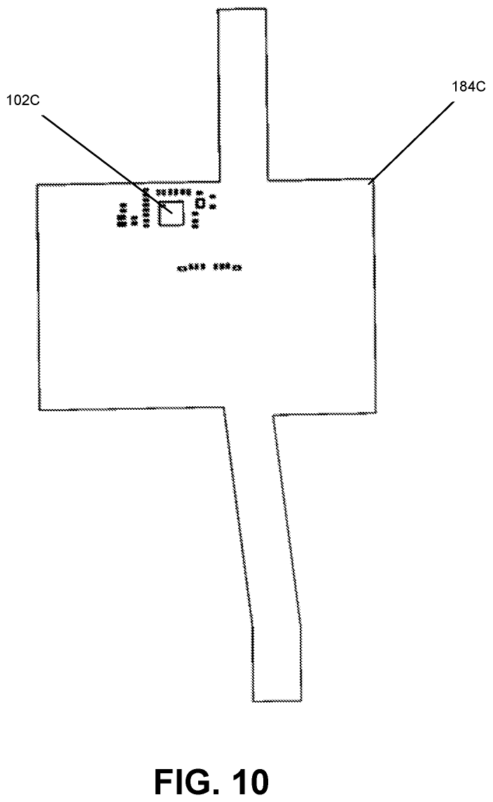

In some embodiments, the liquid fragrance dispenser 100 includes one dispenser controller 102 for controlling the operation of the liquid fragrance dispenser 100. In some embodiments, the liquid fragrance dispenser 100 includes more than one dispenser controller 102, with the dispenser controllers 102 configured to control different functions of the liquid fragrance dispenser 100. In some embodiments, as depicted in FIG. 9, the liquid fragrance dispenser 100 includes a dispenser controller 102A that is mounted on the circuit board 184A, and is configured to control the overall operation of the liquid fragrance dispenser 100. In some embodiments, the dispenser controller 102A is disposed at a bottom end of the body 152 of the housing 150 of the liquid fragrance dispenser 100. In some embodiments, as depicted in FIG. 10, the liquid fragrance dispenser 100 includes a dispenser controller 102C that is mounted on a circuit board 184C, and is configured to detect the presence of a liquid fragrance cartridge 200 and validate the liquid fragrance cartridge 200. In some embodiments, the circuit board 184C is disposed along a longitudinal length of the liquid fragrance dispenser 100, for example, generally parallel to or disposed in opposition to one of the sides of the liquid fragrance dispenser 100. In some embodiments, as depicted in FIG. 7, the liquid fragrance dispenser 100 includes a dispenser controller 102B that is mounted on a circuit board 184B, and is configured to control the operation of the atomizer subsystem 172, for example, activating or deactivating the atomizer subsystem 172, or changing the vibration frequency of the atomizer disc 176. In some embodiments, where the atomizer is received in the cover 154, the circuit board 184B is disposed in the cover 154. In some embodiments, the liquid fragrance dispenser 100 includes a dispenser controller that is configured to control the operation of the lighting subsystem 112, for example, by sending a control command to the lighting subsystem 112 to illuminate a certain colour or animation. In some embodiments, said dispenser controller is disposed in the cover 154. In some embodiments, the liquid fragrance dispenser 100 includes a dispenser controller that is configured to control the operation of the capacitive touch surface 158, for example, by sending a control command to the atomizer subsystem 172 or the lighting subsystem 112, upon detection that the capacitive touch surface 158 is touched. In some embodiments, said dispenser controller is disposed in the cover 154.

FIG. 11 depicts an example configuration of the liquid fragrance dispenser 100. As depicted in FIG. 11, in some embodiments, the liquid fragrance dispenser 100 includes a dispenser controller 102, a communications module 103, and a power module 107. In some embodiments, the liquid fragrance dispenser 100 is connectable to an external power source, for example, via the USB connector 186 and a corresponding connection that connects the USB connector 186 to the external power source. In some embodiments, the external power source can be a power outlet, portable battery, portable generator, external battery, battery dock or charging dock, etc. In some embodiments, for example, the liquid fragrance dispenser 100 is wirelessly connectable to the external power source for wirelessly energizing the liquid fragrance dispenser 100.

In some embodiments, the dispenser controller 102 includes a processor or a central processing unit (CPU), a memory 130 such as a ROM, RAM, persistent memory, or flash memory for storing data, and input or output peripherals. The dispenser controller 102 may act as a central controller for controlling all of the communications of the liquid fragrance dispenser 100, the liquid fragrance cartridge 200, the server 300, and the user equipment 400. In some embodiments, the liquid fragrance dispenser 100 has a liquid fragrance dispenser identifier, such as a Media Access Control (MAC) address paired with a unique serial identification number, which may be stored in the memory 130, for example. The MAC address and serial number uniquely identify the liquid fragrance dispenser 100 on the system 10. In some embodiments, the MAC address is included in each communication between the liquid fragrance dispenser 100 and the server 300, and between the liquid fragrance dispenser 100 and the user equipment 400, to identify the liquid fragrance dispenser 100. In some embodiments, the liquid fragrance dispenser 100 communicates directly with the user equipment 400 via wireless communications, such as with the Wi-Fi or cellular radio function. In some embodiments, the liquid fragrance dispenser 100 may act as a hub to other smart home devices, or other liquid fragrance dispensers 100 or the liquid fragrance cartridge 200, or acting as a hot-spot for the liquid fragrance dispenser 100 or the liquid fragrance cartridge 200 placed in recreation vehicles or mobile homes. In some embodiments, in response to a communication received from the server 300, the dispenser controller 102 communicates with the server 300 using a RESTful API, which is a set of Hypertext Transfer Protocol (HTTP) POST and GET requests. In some embodiments, the dispenser controller 102 communicates with the server 300 with the RESTful API to configure a Wi-Fi (Wireless Fidelity) network. In some embodiments, the dispenser controller 102 communicates with the server 300 using a web sockets, long polling sockets, or message queuing telemetry transport (MQTT).

The dispenser controller 102 communicates with the liquid fragrance cartridge 200, the server 300, and/or the user equipment 400 via the communications module 103. In some embodiments, the dispenser controller 102 receives data from the liquid fragrance cartridge 200 and/or from the sensor subsystem 120, saves the data to a memory, and processes the received data. The data may be real time data or historical data. In some embodiments, the dispenser controller 102 processes the data by, for example, comparing data with one or more preset thresholds. In some embodiments, the dispenser controller 102 processes the data by, for example, calculating or determining an actual environmental condition based on the received data. In some embodiments, for example, the variably controlling of the output of the dispenser 100 includes the dispenser controller 102 being configured to variably control the duration of operation of the atomizer subsystem 172 (e.g. activating the atomizer 174 for a period of time; activating the atomizer 174 for a period of time, and then deactivating the atomizer 174 for a period of time; activating the atomizer 174 for a period of time, then deactivating the atomizer 174 for a period of time, and then re-activating the atomizer 174 for a period of time), or to variably control the atomization output, of the atomizer subsystem 172 to achieve the target environmental fragrance condition based on the actual environmental condition. In some embodiments, the target environmental fragrance condition is achieved further based on the previous atomization output of the atomizer 174. In some embodiments, the dispenser controller 102 sends the results of the processed data to the user equipment 400 via the communication module 103, for example, the Wi-Fi module 110. In some embodiments, the dispenser controller 102, based on the processed data, variably controls an output of the liquid fragrance dispenser 100, for example, by variably controlling the duration of operation or atomization output of the atomizer subsystem 172.

In some embodiments, the dispenser controller 102 is configured to upload the data received from the sensor subsystem 120 or the liquid fragrance cartridge 200, or the processed data based on the received data, to the server 300, through the communication module 103. In some embodiments, the dispenser controller 102 sends data, including the sensed data from the sensor subsystem 120 or from the liquid fragrance cartridge 200, to the server 300 periodically, such as once every hour, to update the server 300 with, for example, the latest temperature and humidity, operational status of the atomizer subsystem 120, the ambient light intensity, proximity of nearby objects, among other information. In some embodiments, such data, for example, detected from the sensor subsystem 120 or from the liquid fragrance cartridge 200, can then be transmitted from the server 300 to the user equipment 400 (e.g. push or pull). In some embodiments, as a default, wireless communications between the liquid fragrance dispenser 100 and the server 300 use a Wi-Fi network, or may bypass (do not use or require) any Wi-Fi Network.

In some examples, the data that is uploaded includes the MAC address and a serial identification number of the liquid fragrance dispenser 100 to identify the liquid fragrance dispenser 100, and the identification for the liquid fragrance cartridge 200, such as the MAC address and the serial number of the liquid fragrance cartridge 200 or the identifier of the liquid fragrance cartridge 200, to identify the liquid fragrance dispenser 100 or the liquid fragrance cartridge 200 that has generated the sensed data or was the origin or source of the data. The data can be contained in one or more communications. Some of the data, for example the identification of the liquid fragrance dispenser 100 or the liquid fragrance cartridge 200 and the MAC address, may be contained in a header of a communication (e.g. HTTP communication), while the data relating to the value of the sensed external variables or the data representative of the liquid fragrance cartridge 200 can be the payload of the HTTP communication. Other example triggers for sending the data are as follows: the dispenser controller 102 may also send the data from the sensor subsystem 120 or the liquid fragrance cartridge 200 when the power loss is detected, when the sensor subsystem 120 recognized by the liquid fragrance dispenser 100 detects a change in the environment, such as temperature or humidity or light intensity, or when the dispenser controller 102 recognizes that the amount of liquid in the liquid fragrance cartridge 200 is below a threshold amount. In some embodiments, the server 300 stores the received data or the processed data from the liquid fragrance dispenser 100. In some embodiments, the server 300 communicates the data or the processed results to the user equipment 400 according to the preference of the user, for example, via emails, push notification, and the like.

In some embodiments, the dispenser controller 102 is configured to control the environment of a premises. For example, based on the data from the sensor subsystem 120 or from the liquid fragrance cartridge 200, the dispenser controller 102 variably controls an output of the liquid fragrance dispenser 100, such as an output from the lighting subsystem 112, a speaker 124, or the atomizer subsystem 172.

In some embodiments, the communication module 103 includes a short range communication that is used to determine that a liquid fragrance cartridge 200 is placed in proximity to the liquid fragrance dispenser 100. In some embodiments, the communication module 103 includes a short range communication that is used to detect or determine that a liquid fragrance cartridge 200 is releasably or removably coupled to the liquid fragrance dispenser 100, for example, the atomizer subsystem 172 of the liquid fragrance dispenser 100. In some embodiments, the dispenser controller 102 is configured to detect the releasable or removable coupling of the liquid fragrance cartridge 200 to the atomizer subsystem 172 using a short range communication protocol. In some embodiments, said short range communication protocol is a radio-frequency identification (RFID) protocol or a near field communication (NFC) protocol. As depicted in FIG. 11, in some embodiments, the communication module 103 include a radiofrequency identification (RF ID) reader 104, an RF module 108, and/or a Wi-Fi module 110. In some embodiments, for example, the communication module 103 includes a Wireless Wide Area Network (WWAN) module, that functions as a wireless communication module, or a wireless communication subsystem, for the liquid fragrance dispenser 100 to access standard wireless communications services, such as communications services provided by GSM, GPRS, 3G, LTE, and 5G wireless networks, and for establishing communication between the liquid fragrance dispenser 100, the liquid fragrance cartridge 200, and the server 300. The dispenser controller 102 controls the communication module 103. In some embodiments, the RF module 108 includes the RF ID reader 104. In some embodiments, the communication module 103 includes a wireless communication subsystem.

The RF ID reader 104 reads or detects the identifier uniquely associated with each of the liquid fragrance cartridge 200, for example, from a RF ID, an NFC tag, and the like, when the liquid fragrance cartridge 200 is tapped to or placed in a close proximity with the liquid fragrance dispenser 100, for example, when the liquid fragrance cartridge 200 is inserted or received in a cavity 188 defined by the housing 150 of the liquid fragrance dispenser 100, as depicted in FIG. 12. In some examples, the RF ID reader 104 sends the identifiers read from the liquid fragrance cartridge 200 to the dispenser controller 102 for the liquid fragrance dispenser 100 to recognize the liquid fragrance cartridge 200 and for the liquid fragrance dispenser 100 to store the identifiers in the memory 130 of the liquid fragrance dispenser 100. The dispenser controller 102 then registers the liquid fragrance cartridge 200 with the liquid fragrance dispenser 100 based on their respective RF identifiers. In other words, with the RF identifiers, the liquid fragrance dispenser 100 recognizes respective a liquid fragrance cartridge 200. The recognizing process will be described in greater detail below. The liquid fragrance cartridge 200 may send its identifier (e.g. alphanumeric 9 digit code or the Media Access Control (MAC) address) to the liquid fragrance dispenser 100 via any near field communications modules, such as infrared or Bluetooth™.

RF module 108 allows the liquid fragrance dispenser 100 to transmit and/or receive data in the form of wireless signals with the corresponding RF module of the liquid fragrance cartridge 200, using for example unlicensed frequency spectrum, for example on 915 MHz band. Example embodiments that refer to the unlicensed frequency spectrum can also be applied to one unlicensed frequency channel. The RF module 108 may include power amplifying circuits for amplifying the RF signals, and frequency modulation circuits for modulating the signals to the selected radio frequency, and antennas for the RF signals to be radiated to the liquid fragrance cartridge 200 or to receive the RF signals from the liquid fragrance cartridge 200. In some embodiments, the data transmitted and/or received between the RF module 108 of the liquid fragrance dispenser 100 and the RF module 208 of the liquid fragrance cartridge 200 is transmitted and/or received via a short range communication protocol, such as a radio-frequency identification (RFID) protocol or a near field communication (NFC) protocol, executed by the RF module 108 and the RF module 208.

The Wi-Fi module 110 provides circuits that enable the liquid fragrance dispenser 100 to use Wi-Fi networks and to transmit data to the server 300 or the liquid fragrance cartridge 200, and to receive data from the liquid fragrance cartridge 200 or from the server 300. In some embodiments, the Wi-Fi module 110 includes a Wi-Fi transceiver. In some embodiments, a user uses the user equipment 400 to configure the Wi-Fi module 110 via the server 300, for example, via a cloud based web-portal. The Wi-Fi configuration process will be described in great detail below.

In some embodiments, the Wi-Fi module 110 scans available Wi-Fi networks, and connects the liquid fragrance dispenser 100 to a selected Wi-Fi network. In some embodiments, the Wi-Fi module 110 detects loss of the Wi-Fi networks and loss of the Internet connection. In some embodiments, the Wi-Fi module 110 makes HTTP request over SSL and open a TCP socket over SSL so that the Wi-Fi module 110 may access a webpage using TCP/IP protocol. In some examples, all of the communications between the liquid fragrance dispenser 100 and the server 300 is encrypted. In some embodiments, the encryption is transport layer security (TLS) encryption.

In some embodiments, the liquid fragrance dispenser 100 activates the Wi-Fi module 110 as the primary communication module when the liquid fragrance dispenser 100 is initialized, for example, powered on.

In some embodiments, the power module 107 includes a power detection circuit, such as a power detector 140, to determine when outlet power is lost. The power detector 140 is a presence/absence power detector 140, in some embodiments, for example. In some embodiments, the power detector 140 measures the specific signal from the outlet (e.g., power, voltage or current). When the input power from the outlet of the premises is lost, drops below a threshold, or is fluctuating, the battery backup 116 is configured to seamlessly supply power to the liquid fragrance dispenser 100, for example, by the rechargeable battery. Typically, the rechargeable battery is capable of supplying power the liquid fragrance dispenser for at least 24 hours. In some embodiments, the dispenser controller 102 reports the remaining power of the battery to the server 300 and the user equipment 400. When the power is lost, the dispenser controller 102 reports the power loss to the server 300 as an alert event, for example via HTTP request and/or to the user equipment 400 via emails, text messages, or push notification. In some embodiments, the liquid fragrance dispenser 100 uses only the Wi-Fi module 110 to transmit the data received from the sensor subsystem 120 or the liquid fragrance cartridge 200 to the server 300, the RF module 108 remains active for receiving messages, such as anomalies, from the sensor subsystem 120 or the liquid fragrance cartridge 200r.

In some embodiments, the power module 107 includes a charging circuit 114, and a battery backup 116. The charging circuit 114 receives the power from an outlet of a premises, converts the received power to appropriate voltage and current, and supplies the converted power to various elements of the liquid fragrance dispenser 100. In some embodiments, the battery backup 116 includes a rechargeable battery, such as a rechargeable Lithium ion battery. In some embodiments, the charging circuit 114 directly supplies the converted power to the battery backup 116 for charging the rechargeable battery, the dispenser controller 102, and communication module 103, the sensor subsystem 120, the atomizer subsystem 172, and the user interface subsystem 155, which includes the lighting subsystem 112 and the speaker 124. In some embodiments, the charging circuit 114 supplies the converted voltage and current to the battery backup 116 for charging the rechargeable battery, and the rechargeable battery of the battery backup 116 supplies power to the liquid fragrance dispenser 100, such as the dispenser controller 102, and communication module 103, the sensor subsystem 120, the atomizer subsystem 172, and the user interface subsystem 155, which includes the lighting subsystem 112 and the speaker 124. In some embodiments, the power module 107 includes a switch to turn on or off of the liquid fragrance dispenser 100.

In some embodiments, when the input power from the power outlet is lost, the liquid fragrance dispenser 100 operates in a sleep mode, in which the dispenser controller 102 turns off the communication module 103 and only activates the one or more elements of the communication module 103 when necessary, and/or periodically.

In some embodiments, the liquid fragrance dispenser 100 includes the user interface subsystem 155, which includes a lighting subsystem 112. The lighting subsystem 112 provides visual indication, for example, the status of the liquid fragrance dispenser 100. The lighting subsystem 112, is, for example, a LED light or an LED light strip. In some embodiments, the dispenser controller 102 controls the colours of the lighting subsystem 112, based on events of the liquid fragrance dispenser 100 or the data received from the sensor subsystem 112 or the liquid fragrance cartridge 200. In some embodiments, different colours of the lighting subsystem 112 indicates different statuses of the liquid fragrance dispenser 100. In some embodiments, the lighting subsystem 112 is green when the liquid fragrance dispenser 100 is connected with both cellular radio network and a Wi-Fi network; the lighting subsystem 112 is yellow when the liquid fragrance dispenser 100 is connected with only the cellular radio network. In some embodiments, different colours are used during the boot up process of the liquid fragrance dispenser 100 to indicate signal strength of the cellular radio network and a Wi-Fi network, so that proper location may be selected to place the liquid fragrance dispenser 100. In some embodiments, when the dispenser controller 102 determines that based on the data received from the sensor subsystem 120 or the liquid fragrance cartridge 200 that an output of the liquid fragrance dispenser 100 is being variably controlled, for example, the duration of operation of the atomizer subsystem 172 or atomization output of the atomizer subsystem 172, based on an actual environmental condition as detected by the sensor subsystem 120, to achieve the target environmental fragrance condition, the dispenser controller 102 sends a control command to the lighting subsystem 112 to cause the lighting subsystem 112 to have a first colour, such as a blue colour. If the dispenser controller 102 determines that the target environmental fragrance condition is being achieved, based on the actual environmental condition, the dispenser controller 102 sends a control command to the lighting subsystem 112 to cause the lighting subsystem 112 to have a second colour, such as a green colour. In some embodiments, when the input power to the liquid fragrance dispenser 100 from the premises is lost, the battery backup 116 supplies the power to the liquid fragrance dispenser 100 and the dispenser controller 102 sends a control command to the lighting subsystem 112 to cause the lighting subsystem 112 to have emit a bright white light, such that the liquid fragrance dispenser 100 functions as a wayfinder or nightlight.

In some embodiments, the liquid fragrance dispenser 100 includes a sensor subsystem 120. The sensor subsystem 120 is in operable communication with the dispenser controller 102, for example, integrated within a same packaging of the liquid fragrance dispenser 100.

An example sensor is a temperature sensor for detecting and transmitting data representative of the ambient temperature to the dispenser controller 102. An example sensor is a humidity sensor for detecting and transmitting data representative of the ambient humidity to the dispenser controller 102. An example sensor is a sensor that is configured to detect the temperature of the liquid fragrance dispenser 100, such as the temperature of the sensor subsystem 120, the atomizer subsystem 172, the dispenser controller 102, or the user interface subsystem 155 of the liquid fragrance dispenser 100, such as a speaker 124, a microphone 126, or a lighting subsystem 112. An example sensor is an accelerometer that is configured to detect the acceleration of the liquid fragrance dispenser 100. An example sensor is an occupancy detection sensor (e.g. time of flight sensor, passive infrared detection sensor, occupancy detection sensor, etc.) that is configured to detect the distance, range, presence, or proximity of an object relative to the liquid fragrance dispenser 100. An example sensor is a light intensity sensor that is configured to detect the intensity of the ambient light.

In some embodiments, the liquid fragrance dispenser 100 includes a speaker 124 for generating audio signals or alerts. In some embodiments, the speaker 124 generates a first audio signal, such as a beep, to indicate that the liquid fragrance cartridge 200 has been detected and recognized by, and the identifier of the liquid fragrance cartridge 200 is temporarily stored in memory or RAM of the liquid fragrance dispenser 100. In some embodiments, the speaker 124 generate a second audio signal to indicate that the liquid fragrance cartridge 200 has been registered with the server 300, and the identifier of the liquid fragrance cartridge 200 has been stored in persistent memory of the liquid fragrance dispenser 100. In some embodiments, the first sound is accompanied by a first visual output, such as a first LED color output from the lighting subsystem 112 of the liquid fragrance dispenser 100, such as green, and the second sound is accompanied by a second visual output from the lighting subsystem 112 of the liquid fragrance dispenser 100, such as a second LED color output (e.g., can also be green). If the liquid fragrance cartridge 200 is not successfully recognized by the liquid fragrance dispenser 100, in some embodiments, the speaker 124 generates an audio signal, and is accompanied by a visual output from the lighting subsystem 112 of the liquid fragrance dispenser 100, such as an LED color output, such as a red colour. In some embodiments, once the liquid fragrance cartridge 200 has been registered with the server 300, the icon of the liquid fragrance cartridge 200 may be displayed on a user interface, such as a dashboard. In some embodiments, all of the audio signals and sounds are the same sound. In some embodiments, the audio signals and the sounds are different sounds.

In some embodiments, the Wi-Fi module 110 of a liquid fragrance dispenser 100 is configured to report signal strength of available Wi-Fi networks to the server 300. The user equipment 400 may use the signal strength information to indicate the signal strength of the available Wi-Fi networks at one location of the premises. In some embodiments, the liquid fragrance dispenser 100 has a display screen or an icon for displaying signal strength of the available Wi-Fi networks at the location of the liquid fragrance dispenser 100, for example for several minutes, so that a user can determine the optimal location for placing the liquid fragrance dispenser 100 in a premises.

In some embodiments, the liquid fragrance dispenser 100 generates an audio and/or a visual signal to indicate the signal strength of the available Wi-Fi networks. In some embodiments, if the signal strength of a Wi-Fi network detected by the Wi-Fi module 110 is below −100 dBm, the lighting subsystem 112, such as an LED, of the liquid fragrance dispenser 100 may be controlled by the dispenser controller 102 to illuminate a red colour. In some embodiments, if the signal strength of a Wi-Fi network detected by the Wi-Fi module 110 is between 85 dBm to −100 dBm, the lighting subsystem 112 is controlled by the dispenser controller 102 to emit a yellow colour. In some embodiments, if the signal strength of a Wi-Fi network detected by the Wi-Fi module 110 is above −85 dBm, the lighting subsystem 112 is controlled by the dispenser controller 102 to emit a green colour. Similarly, the liquid fragrance dispenser 100 is configured to generate different sounds from the speaker 124, for example, from a low volume to a high volume to indicate the strength of the signal strength from low to high.

In some embodiments, the dispenser memory 130 stores information or data representative of a target environmental fragrance condition. In some embodiments, the target environmental fragrance condition is a fragrance level in the room or premises in which the liquid fragrance dispenser 100 is disposed, as perceived by a user of the liquid fragrance dispensing system 10. In some embodiments, the target environmental fragrance condition is an amount of fragrance in the volume of air or a concentration of fragrance in the air. In some embodiments, the environmental condition contributes to an environmental ambiance of a room. In some embodiments, the user of the system 10 can select or choose the target environmental fragrance condition, such as “low”, “medium”, or “high”. In some embodiments, the user of the system 10 can select or choose the target environmental fragrance condition by inputting a control command to the system 10, for example, to the server 300 or the dispenser controller 102, via the user equipment 400 or via actuation of the button 156 or the capacitive touch surface 158. In some embodiments, the target environmental fragrance condition is a personal preference as selected by the user of the system 10.

In some embodiments, the sensor subsystem 120 of the liquid fragrance dispenser 100 is configured to detect a value of an external variable of the environment of the liquid fragrance dispensing system 10, and the dispenser controller 102 is in operable communication with the sensor subsystem 120. In some embodiments, the sensor subsystem 120 of the liquid fragrance dispenser 100 is configured to continuously detect a value of an external variable of the environment of the liquid fragrance dispensing system.

In some embodiments, the dispenser controller 102 is configured to determine a target environmental fragrance condition information, for example, via the memory 130, the user equipment 400, or a user interface subsystem 155, determine an amount of atomization output of the atomizer subsystem 172, determine an actual environmental condition based on the amount of atomization output of the atomizer and the value of the external variable detected by the sensor subsystem 120, and variably controls an output of the liquid fragrance dispenser. In some embodiments, the variably control includes the dispenser controller 102 being configured to variably control the duration of operation of the atomizer 174 at an atomization output to achieve the target environmental fragrance condition based on the actual environmental condition. In some embodiments, the variably control includes the dispenser controller 102 being configured to variably control the atomization output or atomization rate of the atomizer 174 to achieve the target environmental fragrance condition based on the actual environmental condition. In some embodiments, the target environmental fragrance condition is achieved further based on previous atomization output of the atomizer.

In some embodiments, the sensor subsystem 120 continuously detects the value of the external variable, and the liquid fragrance dispensing system 10 uses the continuously detected value of the external variable as an input for modulating the amount of dispersion of the liquid fragrance to maintain a certain environmental condition that is based on the target environmental fragrance condition.

For example, the user sets their target environmental fragrance condition to “high”, for example, in order for the user to clearly detect the fragrance being dispersed by the liquid fragrance dispenser 100 in the room. The dispenser 102 is configured to determine an actual environmental condition based on the atomization output of the atomizer subsystem 172 and the value of the external variable detected by the sensor subsystem 120. Based on the actual environmental condition, the dispenser controller 102 is configured to determine the operating conditions of the atomizer subsystem 172 (e.g. duration of operation of the atomizer subsystem 172, and/or the atomization output of the atomizer subsystem 172) to achieve the target environmental fragrance condition. Based on this determination, the dispenser controller 102 sends a control command to the atomizer subsystem 172 to variably control the duration of operation of the atomizer 174 (e.g. activating the atomizer 174 for a period of time; activating the atomizer 174 for a period of time, and then deactivating the atomizer 174 for a period of time; activating the atomizer 174 for a period of time, then deactivating the atomizer 174 for a period of time, and then re-activating the atomizer 174 for a period of time), or to variably control (e.g. increase, decrease, or maintain) the vibration of the atomizer disc 176, such that the atomizer 174 disperses an amount of the liquid fragrance into the room to achieve the target environmental fragrance condition. In this regard, the liquid fragrance dispensing system 10 is configured to detect a value of an external variable and an existing atomization output, and variably control the atomizer subsystem 172 based on said detection to reflect or achieve the target environmental fragrance condition. In some embodiments, the target environmental fragrance condition is achieved further based on the previous atomization output of the atomizer 174.

In some embodiments, the sensor subsystem 120 includes a temperature sensor, the actual environmental condition includes temperature, and the target environmental fragrance condition includes temperature. In such embodiments, the dispenser controller 102 is configured to receive, from the temperature sensor, data representative of the temperature detected by the temperature sensor, determine the actual environmental condition based on the data representative of the atomization output of the atomizer subsystem 172 and the temperature detected by the temperature sensor, and variably control the output of the atomizer subsystem 172 to achieve the target environmental fragrance condition based on the actual environmental condition.

In some embodiments, the sensor subsystem 120 includes a humidity sensor, the actual environmental condition includes humidity, and the target environmental fragrance condition includes temperature. In such embodiments, the dispenser controller 102 is configured to receive, from the humidity sensor, data representative of the humidity detected by the humidity sensor, determine the actual environmental condition based on the data representative of the atomization output of the atomizer subsystem 172 and the humidity detected by the humidity sensor, and variably control the output of the atomizer subsystem 172 to achieve the target environmental fragrance condition based on the actual environmental condition.

In some embodiments, the system comprises an accelerometer. In some embodiments, for example, the sensor subsystem 120 includes an accelerometer. In such embodiments, the dispenser controller 102 is configured to determine acceleration threshold information, receive acceleration threshold information from the dispenser memory 130, receive, from the accelerometer, data representative of the acceleration of the liquid fragrance dispenser 100 as detected by the accelerometer, perform a comparison of the acceleration threshold information and the acceleration of the liquid fragrance dispenser 100, and deactivate the atomizer subsystem 172 based on the comparison. In this regard, the dispenser controller 102 is configured to deactivate the atomizer subsystem 172, for example, when the liquid fragrance dispenser 100 is falling or has fallen from an upright operational position.

In some embodiments, the liquid fragrance dispenser 100 comprises the user interface subsystem 155 having a lighting subsystem 112, and the sensor subsystem 120 includes an occupancy detection sensor. In some embodiments, the occupancy detection sensor includes a time of flight sensor. In some embodiments, the time of flight sensor generates a signal representative of a proximity of an object, or the distance between the sensor and the object. The distance between the time of flight sensor and the object, D, is calculated at time T with electromagnetic radiation at light speed c, is D=cT. The time of flight sensor emits radiation, which travels towards the object and is then reflected back by the object to the sensor receiver. The distance covered is now 2D at time T. The relationship can be written as:

$$D = \frac{cT}{2}$$

In such embodiments, the dispenser controller 102 is configured to receive, from the time of flight sensor, data representative of an object's proximity detected by the time of flight sensor; and wherein the variably controlling an output of the dispenser 100 includes the dispenser controller 102 being configured to variably activate the light output of the lighting subsystem 112 based on the object's proximity. In this regard, the liquid fragrance dispenser 100 operates when a user is proximate the liquid fragrance dispenser 100 within a distance threshold (e.g. when the user is home), and the liquid fragrance dispenser 100 does not when a user is outside of the distance threshold (e.g. when the user is not home). In some embodiments, when the dispenser controller 102 determines that there is movement within a distance threshold of the liquid fragrance dispenser 100 (e.g. the user is proximate the liquid fragrance dispenser 100), the dispenser controller 102 sends a control command to the atomizer subsystem 172 to activate or variably control the atomizer 174 to disperse a fragrance in the room. In some embodiments, when the dispenser controller 102 determines that there is movement within a distance threshold of the liquid fragrance dispenser 100 (e.g. the user is proximate the liquid fragrance dispenser 100), the dispenser controller 102 sends a control command to the lighting subsystem 112 to for the lighting subsystem 112 to illuminate a light. In some embodiments, when the dispenser controller 102 determines that there is no movement within a distance threshold of the liquid fragrance dispenser 100 (e.g. the user is not proximate the liquid fragrance dispenser 100), the dispenser controller 102 sends a control command to the atomizer subsystem 172 to deactivate or variably control the atomizer 174, such that a fragrance is not dispersed into the room, or the dispersion rate of the fragrance is reduced.

In some embodiments, the occupancy detection sensor includes a passive infrared detection sensor. In some embodiments, the passive infrared detection sensor measures infrared radiation radiating from objects in the field of view of the sensor, and generates a signal representative of a proximity or presence of an object in the field of view of the passive infrared detection sensor. In some embodiment, the passive infrared detection sensor detects changes in the amount of infrared radiation that is directed at or detected by the passive infrared detection sensor. In some embodiments, said changes in the amount of infrared radiation is representative of the temperature and surface characteristics of objects in the field of view of the sensor, and representative of a person in the field of view of the passive infrared detection sensor. For example, when a person walks into and then out of the field of view of the passive infrared detection sensor, the sensor detects a temperature change, namely, an increase of temperature from room temperature to body temperature, and then a decrease of temperature from body temperature to room temperature. Based on this detection of temperature change, the passive infrared detection sensor generates a signal representative of the temperature change and transmits the signal to the dispenser controller 102. The signal is representative of an indication that movement, and therefore, the presence or proximity, of an object, such as a person, is detected in the field of view of the passive infrared detection sensor. In such embodiments, the dispenser controller 102 is configured to receive, from the passive infrared detection sensor, data representative of an object's proximity or presence detected by the passive infrared detection sensor; and wherein the variably controlling an output of the dispenser 100 includes the dispenser controller 102 being configured to variably activate the light output of the lighting subsystem 112 based on the object's proximity or presence. In this regard, the liquid fragrance dispenser 100 operates when a user is proximate the liquid fragrance dispenser 100 within a distance threshold based on the field of view of the passive infrared detection sensor (e.g. when the user is home), and the liquid fragrance dispenser 100 does not when a user is outside of said distance threshold (e.g. when the user is not home). In some embodiments, when the dispenser controller 102 determines that there is movement within a distance threshold of the liquid fragrance dispenser 100 (e.g. the user is within the field of view of the passive infrared detection sensor and proximate the liquid fragrance dispenser 100), the dispenser controller 102 sends a control command to the atomizer subsystem 172 to activate or variably control the atomizer 174 to disperse a fragrance in the room. In some embodiments, when the dispenser controller 102 determines that there is movement within a distance threshold of the liquid fragrance dispenser 100 (e.g. the user is proximate the liquid fragrance dispenser 100), the dispenser controller 102 sends a control command to the lighting subsystem 112 to for the lighting subsystem 112 to illuminate a light. In some embodiments, when the dispenser controller 102 determines that there is no movement within a distance threshold of the liquid fragrance dispenser 100 (e.g. the user is not proximate the liquid fragrance dispenser 100), the dispenser controller 102 sends a control command to the atomizer subsystem 172 to deactivate or variably control the atomizer 174, such that a fragrance is not dispersed into the room, or the dispersion rate of the fragrance is reduced.

In some embodiments, the occupancy detection sensor includes a microwave radar sensor. In some embodiments, the microwave radar sensor continuously emits microwave radiation, and measures the amount of time that the emitted microwave radiation returns to the sensor after being reflected by an object. The microwave radar sensor measures changes in the amount of time that the emitted microwave radiation returns to the sensor. In some embodiments, the microwave radar sensor measures a phase change of the microwave radiation that has returned to the sensor. In some embodiments, said changes in the amount of time for the emitted microwave radiation to return to the sensor, or the phase change of the returned microwave radiation, is representative of a person in the field of view of the microwave radar sensor. For example, when a person walks into and then out of the field of view of the microwave radar sensor, the sensor detects a time change, namely, a change in the amount of time that the emitted microwave radiation returns to the sensor, compared to the amount of time that the emitted microwave radiation returns to the sensor if the person is not in the field of view of the sensor. Based on this detection of time change, the microwave radar sensor generates a signal corresponding to the time change and transmits the signal to the dispenser controller 102. The signal is representative of an indication that the presence or proximity of an object, such as a person, is detected in the field of view of the microwave radar sensor. In such embodiments, the dispenser controller 102 is configured to receive, from the microwave radar sensor, data representative of an object's proximity or presence detected by the microwave radar sensor; and wherein the variably controlling an output of the dispenser 100 includes the dispenser controller 102 being configured to variably activate the light output of the lighting subsystem 112 based on the object's proximity or presence. In this regard, the liquid fragrance dispenser 100 operates when a user is proximate the liquid fragrance dispenser 100 within a distance threshold based on the field of view of the microwave radar sensor (e.g. when the user is home), and the liquid fragrance dispenser 100 does not when a user is outside of said distance threshold (e.g. when the user is not home). In some embodiments, when the dispenser controller 102 determines that there is movement within a distance threshold of the liquid fragrance dispenser 100 (e.g. the user is within the field of view of the microwave radar sensor and proximate the liquid fragrance dispenser 100), the dispenser controller 102 sends a control command to the atomizer subsystem 172 to activate or variably control the atomizer 174 to disperse a fragrance in the room. In some embodiments, when the dispenser controller 102 determines that there is movement within a distance threshold of the liquid fragrance dispenser 100 (e.g. the user is proximate the liquid fragrance dispenser 100), the dispenser controller 102 sends a control command to the lighting subsystem 112 to for the lighting subsystem 112 to illuminate a light. In some embodiments, when the dispenser controller 102 determines that there is no movement within a distance threshold of the liquid fragrance dispenser 100 (e.g. the user is not proximate the liquid fragrance dispenser 100), the dispenser controller 102 sends a control command to the atomizer subsystem 172 to deactivate or variably control the atomizer 174, such that a fragrance is not dispersed into the room, such that the dispersion rate of the fragrance is reduced.

In some embodiments, based on the data representative of the amount of time for the emitted microwave radiation to return to the sensor, the dispenser controller 102 is configured to determine the size of the room in which the liquid fragrance dispenser 100 is disposed. For example, when the amount of time for the emitted microwave radiation to return to the sensor is below a first threshold, the dispenser controller 102 is configured to determine that the liquid fragrance dispenser 100 is disposed in a relatively small room. For example, when the amount of time for the emitted microwave radiation to return to the sensor is above a first threshold and below a second threshold, the second threshold being higher than the first threshold, the dispenser controller 102 is configured to determine that the liquid fragrance dispenser 100 is disposed in a relatively medium-sized room. For example, when the amount of time for the emitted microwave radiation to return to the sensor is above the second threshold, the dispenser controller 102 is configured to determine that the liquid fragrance dispenser 100 is disposed in a relatively large room. In some embodiments, data representative of the room size is input from the user interface subsystem 155, for example, the button 156, the capacitive touch surface 158, or the microphone 126. In some embodiments, data representative of the room size is input from the user equipment 400. Based on the room size, whether determined by the dispenser controller 102 or input by the user, the dispenser controller 102 is configured to variably control the output of the atomizer subsystem 172 to achieve the target environmental fragrance condition.

In some embodiments, the sensor subsystem 120 includes a volatile organic compound sensor, the actual environmental condition includes air quality based on the amount of volatile organic compounds in the air, and the target environmental fragrance condition includes air quality based on the amount of volatile organic compounds in the air. In some embodiments, the dispenser controller 102 is configured to receive, from the volatile organic compound sensor, data representative of the air quality by the volatile organic compound sensor. In some embodiments, the dispenser controller 102 is configured to determine if the air quality is below a threshold air quality. When the dispenser controller 102 determines that the air quality is below a threshold air quality, the dispenser controller 102 sends a control command to the lighting subsystem 112 for the lighting subsystem 112 to illuminate a light (e.g. a red light) to alert the user. In some embodiments, when the dispenser controller 102 determines that the air quality is below a threshold air quality, the dispenser controller 102 sends a control command to the user equipment 400 for the user equipment 400 to display a warning, for example, on the screen of the user equipment 400, to alert the user. In some embodiments, the dispenser controller 102 is configured to determine the actual environmental condition based on the data representative of the atomization output of the atomizer subsystem 172 and the air quality detected by the volatile organic compound sensor, and variably control the output of the atomizer subsystem 172 to achieve the target environmental fragrance condition based on the actual environmental condition.

In some embodiments, for example, the liquid fragrance dispenser 100 includes an air ionizer 128. As depicted in FIG. 11, the air ionizer 128 is in operable communication with the dispenser controller 102. In some embodiments, the air ionizer 128 is configured to produce negative ions that are dispersed in the environment, which, in some embodiments, improves the mood of a user. In some embodiments, during activation or variable control of the atomizer subsystem 172, the dispenser controller 102 sends a control command to the air ionizer 128 to activate the air ionizer 128 to produce negative ions.

In some embodiments, the dispenser controller 102, the server 300, and the user equipment 400 are co-operatively configured to provide geo-fencing functionality for the liquid fragrance dispensing system 10. In some embodiments, the server 300 or the user equipment 400 is configured to receive data representative of GPS coordinates of the liquid fragrance dispenser 100, for example, by user input via the user equipment 400. In some embodiments, the server 300 or user equipment 400 are configured to determine, based on the GPS coordinates of the user equipment 400 and the GPS coordinates of the liquid fragrance dispenser 100, the proximity of the user equipment 400 relative to the liquid fragrance dispenser 100, and, based on said determination, send a control command to the dispenser controller 102 to variably control the output of the dispenser 100. In some embodiments, the variably controlling the output of the dispenser 100 includes variably controlling the duration of operation of the atomizer 174, or variably controlling the atomization output of the atomizer 174. In this regard, for example, the server 300 or the user equipment 400 uses the GPS coordinates of the user equipment 400 and the liquid fragrance dispenser 100 to confirm that the user is proximate the liquid fragrance dispenser 100, before activating the atomizer subsystem 172 to disperse a liquid fragrance, such that the liquid fragrance dispenser 100 is operational only when a user is nearby. In some embodiments, for example, the liquid fragrance dispensing system 10 uses the GPS coordinates of the user equipment 400 and the GPS coordinates of the liquid fragrance dispenser 100 to variably control the duration of operation or atomization output of the atomizer subsystem 172. For example, based on the GPS coordinates of the user equipment 400 and the GPS coordinates of the liquid fragrance dispenser 100, if the user is far from the liquid fragrance dispenser 100, the atomizer 174 is not activated, or activated with a relatively short duration or a relatively low atomization output. As another example, based on the GPS coordinates of the user equipment 400 and the GPS coordinates of the liquid fragrance dispenser 100, if the user is proximate to the liquid fragrance dispenser 100, the atomizer 174 is activated, or activated with a relatively long duration or a relatively high atomization output.

In some embodiments, based on the times and durations that the atomizer subsystem 172 is activated or deactivated, the dispenser controller 102, the server 300, and/or the user equipment 400 can automatically determine a schedule of operation of the liquid fragrance dispenser 100, for example, via machine learning. In some embodiments, the user is away from home at certain periods of the day (e.g. the user is at work), and the user is at home during other certain periods of the day. In such embodiments, via machine learning, the dispenser controller 102, the server 300, and/or the user equipment 400 determines that the liquid fragrance dispenser 100 is operational only during certain periods of time during the day, and is not operational during other certain periods of time during the day. In some embodiments, the dispenser controller 102, the server 300, and/or the user equipment 400 performs a comparison of the schedule of operation and the GPS coordinates of the user equipment 400, and based on the comparison, determines if the atomizer subsystem 172 should be activated. In this regard, the liquid fragrance dispensing system 10 is configured to automatically activate the liquid fragrance dispenser 100 by learning the schedule of operation of the liquid fragrance dispenser 100, and by determining that the user is proximate the liquid fragrance dispenser 100.

In some embodiments, the liquid fragrance dispenser 100 comprises the user interface subsystem 155 having the lighting subsystem 112 operating at a lighting intensity, and the sensor subsystem 120 includes an ambient light sensor. In some embodiments, the controller 102 is configured to receive, from the ambient light sensor, data representative of the ambient light detected by the ambient light sensor, and variably control the lighting intensity of the lighting subsystem 112 in general proportion to the intensity of the ambient light. In some embodiments, the dispenser controller 102 controls the lighting intensity of the lighting subsystem 112, such that the lighting subsystem 112 emits a light of desired intensity. For example, the dispenser controller 102 may increase the intensity of the emitted light if the dispenser controller 102 determines that it is dark, the dispenser controller 102 may decrease the intensity of the emitted light if the dispenser controller 102 determines that it is bright, or the dispenser controller 102 may deactivate the lighting subsystem 112 when it is night time.

In some embodiments, the sensor subsystem 120 includes one or more sensors that is configured to detect the operating conditions of the liquid fragrance dispenser 100, such as the temperature of the dispenser controller 102. In such embodiments, the dispenser controller 102 is configured to receive operating condition threshold information from the dispenser memory 130, receive, from said one or more sensors, data representative of the operating conditions of the liquid fragrance dispenser 100, perform a comparison of the operating condition threshold information and the operating conditions of the liquid fragrance dispenser 100, and deactivate the atomizer subsystem 172 or not activate the atomizer subsystem 172 based on the comparison. In this regard, the dispenser controller 102 is configured to deactivate the atomizer subsystem 172, for example, when the liquid fragrance dispenser 100 is operating at an unsafe temperature.

In some embodiments, it is the server 300 that processes the data as detected by the sensor subsystem 120, performs the comparisons where applicable, and variably controls the output of the liquid fragrance dispenser 100. In some embodiments, for example, the server 300 is configured to determine a target environmental condition, determine an amount of atomization output of the atomizer 172, and receive data representative of the value of the external variable sensed by the sensor subsystem 102. In some embodiments, for example, said information and data is transmitted to the server 300 by the dispenser controller 102. The server 300 is further configured to receive said data and information, and determine an actual environmental condition based on the data representative of the atomization output of the atomizer subsystem 172 and the value of the external variable detected by the sensor subsystem 120, and sends a control command to the dispenser controller 102 to variably control the output (e.g. duration of operation, and/or atomization output) of the atomizer subsystem 172 to achieve the target environmental fragrance condition based on the actual environmental condition.

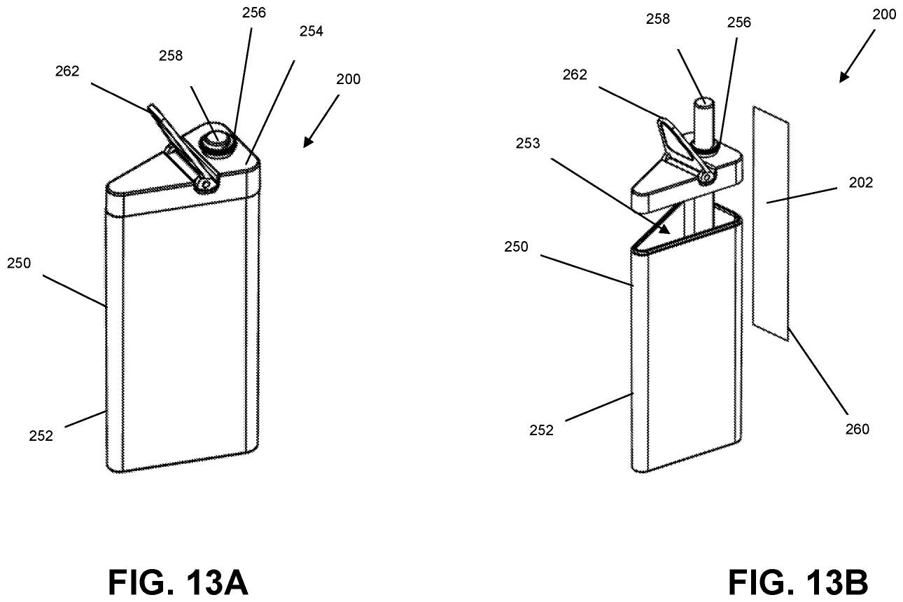

In some embodiments, as depicted in FIG. 12, FIG. 13A, and FIG. 13B, the liquid fragrance dispersing system 10 includes a liquid fragrance cartridge 200. As depicted in FIG. 13A and FIG. 13B, the liquid fragrance cartridge 200 includes a housing 250. In some embodiments, the housing 250 includes a body 252 and a cover 254. In some embodiments, the body 252 and the cover 254 are releasably couplable, for example, by friction fit, interference fit, snap fit, and the like. In some embodiments, the body 252 and the cover 254 are releasably couplable with a fastener. In some embodiments, the body 252 and the cover 254 are releasably couplable with a hinge. In some embodiments, the body 252 is a container 252 that defines a cavity 253 to contain a liquid fragrance. In some embodiments, the housing 250 is manufactured using a plastic such as polyethylene terephthalate (“PET”) that is formed, for example blow moulded or injection moulded, and holds a liquid fragrance, such as a mixture of one or more essential oils and a carrier, to be atomized. In some embodiments, the housing 250 is manufactured using a flexible material to avoid depressurization of the liquid fragrance cartridge 200 during atomization of the liquid fragrance.

In some embodiments, the housing 250 of the liquid fragrance cartridge 200 is shaped generally similarly to the housing 150 of the liquid fragrance dispenser 100 to have a pleasant aesthetic. As depicted in FIG. 12, FIG. 13A, and FIG. 13B, the housing 250 has a triangular prism shape generally similar to that of the housing 150.

In some embodiments, the liquid fragrance cartridge 200 includes a port 256 for providing fluid communication to the cavity 253 of the container 252. As depicted in FIG. 13A and FIG. 13B, in some embodiments, the port 256 is defined on the cover 254.

In some embodiments, the liquid fragrance cartridge 200 includes a wick 258. The wick 258 may be a cotton wick or a synthetic wick. In some embodiments, the wick 258 is sufficiently porous such that the wick 258 draws the liquid fragrance out of the cavity 253 and to the exposed end of the wick 258, as depicted in FIG. 13A and FIG. 13B, at a sufficient fluid flow (e.g. 30 mm per hour).

In some embodiments, the liquid fragrance cartridge 200 includes a cartridge controller 202 that is in operable communication with a cartridge memory 216, for executing instructions stored in the cartridge memory 216 that, when executed, causes the cartridge controller 202 to transmit data representative of the liquid fragrance cartridge 200. In some embodiments, for example, the cartridge controller 202 is for executing instructions stored in the cartridge memory 216 that, when executed, causes the cartridge controller to wirelessly transmit data representative of the liquid fragrance cartridge 200 to the liquid fragrance dispenser 100. In some embodiments, the data representative of the liquid fragrance cartridge 200 includes data representative of the characteristics of the liquid fragrance cartridge 200. As depicted in FIG. 13B, in some embodiments, the cartridge controller 202 is mounted to a circuit board 260, which is mounted to the body 252 of the liquid fragrance cartridge 200.

In some embodiments, the cartridge memory 216 includes information representative of an identifier of a liquid fragrance contained in the liquid fragrance cartridge 200. In some embodiments, the identifier is a code on an RFID chip, an NFC tag, a memory coupled to an interface, a label, and the like. In some embodiments, the cartridge controller 202 is configured to transmit said identifier information to the dispenser controller 102, such that the dispenser controller 102 can automatically determine the liquid fragrance loaded in the liquid fragrance dispenser 100.

In some embodiments, the liquid fragrance cartridge 200 is releasably couplable to the atomizer subsystem 172, for example, the atomizer 174, of the liquid fragrance dispenser 100. In such embodiments, while the liquid fragrance cartridge 200 is releasably coupled to the atomizer subsystem 172 of the liquid fragrance dispenser 100, the liquid fragrance contained in the liquid fragrance cartridge 200 is in fluid communication with the atomizer 174 for atomization by the atomizer 174.

In some embodiments, as depicted in FIG. 13A and FIG. 13B, the liquid fragrance cartridge 200 includes a tab 262. As depicted in FIG. 13A and FIG. 13B, in some embodiments, the tab 262 is connected to the cover 254, for example, pivotably connected to the cover 254. In some embodiments, the tab 262 is connected to the housing 250. The tab 262 is displaceable between a first position, and a second position, as depicted in FIGS. 13A and 13B. In some embodiments, the tab 262 is displaceable between the first position and the second position, for example, via an application of a force, such as a pushing force or a pulling force, to the tab 262. In the first position, the tab 262 is disposed relative to the cover 254 or the housing 250 such that the tab 262 may not be gripped, for example, by a user. For example, while the tab 262 is in the first position, the tab 262 is resting on or supported by the cover 254, or otherwise not extending outwardly relative to the cover 254. In some embodiments, to maintain the aesthetic appearance of the liquid fragrance dispenser 200, the tab 262 has a shape such that, while the tab 262 is in the first position, the tab 262 and the cover 254 are co-operatively configured to provide the aesthetic of a continuous, one-piece, component. In the second position, the tab 262 is disposed relative to the cover 254 or the housing 250 such that the tab 262 may be gripped, for example, by a user. As depicted in FIGS. 13A and 13B, while the tab 262 is in the second position, the tab 262 is extending outwardly relative to the cover 254, such that the tab 262 may be gripped or pinched. While the liquid fragrance cartridge 200 is disposed in the cavity 188, and the tab 262 is in the second position and is being gripped or pinched, in response to a pulling force applied to the tab 262, the liquid fragrance cartridge 200 may be pulled out of the cavity 188.

In some embodiments, as depicted in FIG. 12, the body 152 defines a cavity 188 for receiving the liquid fragrance cartridge 200. In some embodiments, the body 152 defines a cavity 188 for receiving each of the liquid fragrance cartridges 200, for example, two liquid fragrance cartridges. In some embodiments, for example, the cavity 188 is for the releasably coupling of the liquid fragrance cartridge 200 to the atomizer 174. In some embodiments, the shape of the cavity 188 is similar to the shape of the housing 250 of the liquid fragrance cartridge 200, such that lateral movement of the liquid fragrance cartridge 200 while the liquid fragrance cartridge 200 is received in the cavity 188 is limited.

In some embodiments, as depicted in FIG. 12, the atomizer subsystem 172 is received in the cover 154, and the liquid fragrance cartridge 200 is received in the cavity 188 defined by the body 152.

In such embodiments, the cover 154 is displaceable between a first position, as depicted in FIG. 12, and a second position, as depicted in FIG. 2. In the first position, the atomizer subsystem 172 is disposed relative to the cavity 188 such that, while the liquid fragrance cartridge 200 is received in the cavity 188, fluid communication between the liquid fragrance contained in the cartridge 200 and the atomizer 174 is absent. In the second position, the atomizer subsystem 174 is disposed relative to the cavity 188 such that, while the cartridge 200 is received in the cavity 188, fluid communication between the liquid fragrance and the atomizer 174 is present. In the second position, in some embodiments, one end of the wick 258 is extending into the cavity 253 and in liquid communication with the liquid fragrance contained in the cavity 253, and the exposed end of the wick 258 is in liquid communication with the atomizer disc 174, and drawing the liquid fragrance from the cavity 253 to the atomizer disc 174 at a sufficient flow rate, for example, 30 mm per hour.

In some embodiments, the liquid fragrance dispenser 100 has a spring-loaded feature, such as a spring-loaded piston a spring-loaded latch, for releasably coupling the liquid fragrance dispenser 100 and the liquid fragrance cartridge 200. The spring-loaded feature is displaceable between a first position and a second position. In the first position, the spring-loaded feature is extended, and in the second position, the spring-loaded feature is retracted. In some embodiments, while the spring-loaded feature is in the first position, after the liquid fragrance cartridge 200 is inserted into the cavity 188, an upper portion of the liquid fragrance cartridge 200 extends beyond the cavity 188 (see, for example, the left liquid fragrance cartridge 200 depicted in FIG. 12). In some embodiments, while the liquid fragrance cartridge 200 is received in the cavity 188, a downward force is applied to the liquid fragrance cartridge 200, such that the spring-loaded feature is displaced from the first position to the second position, and the spring-loaded feature is retracted. With the spring-loaded feature in the retracted position, the liquid fragrance cartridge 200 is received in the cavity 188, with the housing 250 of the liquid fragrance cartridge 200 received in the cavity 188, to releasably couple the liquid fragrance dispenser 100 and the liquid fragrance cartridge 200. To remove the liquid fragrance cartridge 200, a downward force is applied to the liquid fragrance cartridge 200, such that the spring-loaded feature is displaced from the second position to the first position, and the spring-loaded feature is extended, such that the upper portion of the liquid fragrance cartridge 200 extends beyond the cavity 188. A user may grip the portion of the liquid fragrance cartridge 200 that extends beyond the cavity 188 to remove the liquid fragrance cartridge 200 from the cavity 188.

As depicted in FIG. 12, a magnet 186 is disposed in the cover 154. The magnet 186 is configured to attract to the body 152, or a magnet disposed in the body 152, while the cover is disposed in the second position, in order to releasably couple the cover 154 and the body 152 while the cover 154 is in the second position. In some embodiments, the liquid fragrance dispenser 100 includes a sensor that is configured to detect that the magnet 186 is releasably coupling the cover 154 and the body 152. Said sensor is configured to send a signal representative of the magnet 186 not releasably coupling the cover 154 and the body 152 (e.g. the cover 154 is disposed in the first position, or that the cover 154 is open) to the dispenser controller 102. In response, the dispenser controller 102 is configured to send a control command to the atomizer subsystem 172 to deactivate the atomizer subsystem 172. In this regard, the liquid fragrance dispenser 100 is configured to deactivate while the cover 154 is disposed in the first position, or when the cover 154 is open.

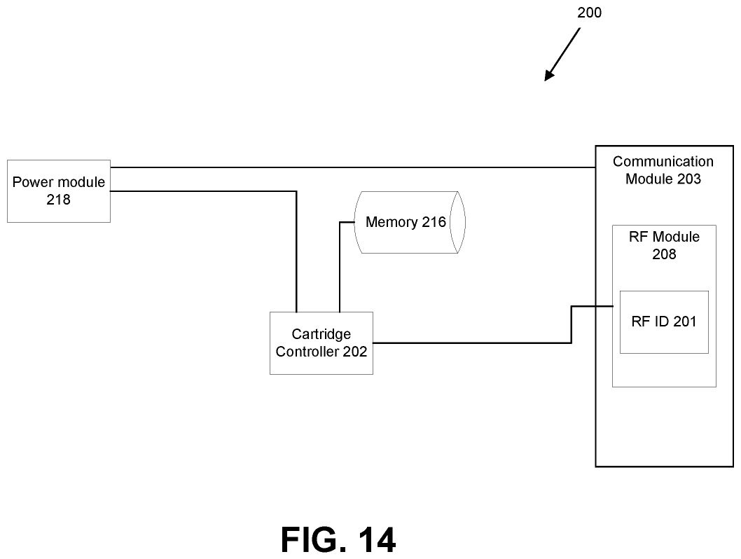

FIG. 14 illustrates an example liquid fragrance cartridge 200. As depicted, in some embodiments, the liquid fragrance cartridge 200 includes an RF ID 201, a cartridge controller 202, a communications module 203, a memory 216, and a power module 218. The cartridge controller 202 is connected with and controls the communication module 203. The liquid fragrance cartridge 200 includes a unique liquid fragrance cartridge ID, such as an alphanumeric 9 digit code or a MAC address of the liquid fragrance cartridge 200. In some embodiments, cartridge controller 202 transmits data representative of the liquid fragrance cartridge 200, which in some embodiments, includes the liquid fragrance cartridge ID, to the dispenser controller 102, periodically or real time.

The RF ID 201 contains an identifier uniquely associated with the liquid fragrance cartridge 200. When the liquid fragrance cartridge 200 is placed at a close proximity of the liquid fragrance dispenser 100, for example, received in the cavity 188 defined by the body 152 of the liquid fragrance dispenser 100, the identifier of the RF ID 201 is read by the RF ID reader 104 of the liquid fragrance dispenser 100. The RF ID reader 104 works on the same frequency as the RF ID. The RF ID reader 104 sends the identifier to the dispenser controller 102. The dispenser controller 102 associates the liquid fragrance cartridge 200 with the identifier. In some embodiments, the RF ID 201 is an NFC tag. The recognizing process will be described below.

In some embodiments, a liquid fragrance cartridge is removed from the liquid fragrance dispenser 100. The user equipment 400, via the server 300, sends a text message to the liquid fragrance dispenser 100 to remove a liquid fragrance cartridge 200 with a specified cartridge identifier.

In some embodiments, the cartridge controller 202 include a processor/a CPU, a memory such as RAM, and input/output peripherals. In some examples, the cartridge controller 202 receives commands from the dispenser controller 102 of the liquid fragrance dispenser 100 via the communications module 203. The cartridge controller 202 then implement the commands from the dispenser controller 102 of the liquid fragrance dispenser 100.

In some embodiments, the memory 216 is substantially similar to the memory 130. In some embodiments, the memory 216 is rewritable memory. The cartridge memory 216 is for storing information representative of the liquid fragrance cartridge 200. In some embodiments, the cartridge controller 202 is in operable communication with the cartridge memory 216, for executing instructions stored in the cartridge memory 216 that, when executed, causes the cartridge controller 202 to transmit data representative of the liquid fragrance cartridge 200. In some embodiments, the cartridge controller 202 is transmitting data representative of the liquid fragrance cartridge 200 in response to a control command from the dispenser controller 102. In some embodiments, the data representative of the liquid fragrance cartridge 200 includes data representative of the characteristics of the liquid fragrance cartridge 200.

In some embodiments, the cartridge memory 216 stores information representative of a fluid type of the liquid fragrance contained in the liquid fragrance cartridge 200, and the cartridge controller 202 is configured to transmit data representative of the fluid type of the liquid fragrance. In some embodiments, for example, the data transmitted by the cartridge controller 202 includes transmitting data representative of the fluid type of the liquid fragrance.

In some embodiments, the cartridge memory 216 stores information representative of a rating on an odor intensity scale of the liquid fragrance contained in the liquid fragrance cartridge 200, and the cartridge controller 202 is configured to transmit data representative of the rating on the odor intensity scale of the liquid fragrance. In some embodiments, for example, the data transmitted by the cartridge controller 202 includes transmitting data representative of the rating on the odor intensity scale of the liquid fragrance.

In some embodiments, the cartridge memory 216 stores information representative of a fragrance decay rate (e.g. fragrance half life) of the liquid fragrance contained in the liquid fragrance cartridge 200, and the cartridge controller 202 is configured to transmit data representative of the half life of the liquid fragrance. In some embodiments, for example, the data transmitted by the cartridge controller 202 includes transmitting data representative of the half life of the liquid fragrance. For example, some types of liquid fragrance may last longer than others.

In some embodiments, the cartridge memory 216 stores information representative of a concentration of a solute in the liquid fragrance contained in the liquid fragrance cartridge 200, and the cartridge controller 200 is configured to transmit data representative of the concentration of the solute in the liquid fragrance. In some embodiments, for example, the data transmitted by the cartridge controller 202 includes transmitting data representative of the concentration of the solute in the liquid fragrance.

In this regard, in some examples the cartridge memory 216 stores information representative of a strength of the liquid fragrance contained in the liquid fragrance cartridge 200.

In some embodiments, the cartridge memory 216 stores information representative of a viscosity of the liquid fragrance contained in the liquid fragrance cartridge 200, and the cartridge controller 202 is configured to transmit data representative of the viscosity of the liquid fragrance. In some embodiments, for example, the data transmitted by the cartridge controller 202 includes transmitting data representative of the viscosity of the liquid fragrance.

In some embodiments, the cartridge memory 216 stores information representative of a date of manufacturing of the liquid fragrance contained in the liquid fragrance cartridge 200, and the cartridge controller 202 is configured to transmit data representative of the date of manufacturing of the liquid fragrance. In some embodiments, for example, the data transmitted by the cartridge controller 202 includes transmitting data representative of the date of manufacturing of the liquid fragrance.

In some embodiments, the cartridge memory 216 stores information representative of an expiration date of the liquid fragrance contained in the liquid fragrance cartridge 200, and the cartridge controller 202 is configured to transmit data representative of the expiration date of the liquid fragrance. In some embodiments, for example, the data transmitted by the cartridge controller 202 includes transmitting data representative of the expiration date of the liquid fragrance.

In some embodiments, the cartridge memory 216 stores information representative of a unique identification of the liquid fragrance cartridge 200, and the cartridge controller 202 is configured to transmit data representative of the unique identification of the liquid fragrance cartridge 200. In some embodiments, for example, the data transmitted by the cartridge controller 202 includes transmitting data representative of the unique identification of the liquid fragrance.

In some embodiments, the cartridge controller 202 is configured to receive data representative of confirmation of validity of the unique identification of the liquid fragrance cartridge, for example, that is validated by the dispenser controller 102 or by the server 300.

In some embodiments, the cartridge memory 216 stores information representative of an amount of liquid fragrance in the liquid fragrance cartridge 200, and the cartridge controller 202 is configured to transmit data representative of the amount of liquid fragrance in the liquid fragrance cartridge 200.

In some embodiments, the cartridge controller 202 is configured to receive data representative of run time and atomization output of an atomizer subsystem, for example, the atomizer subsystem 172 of the liquid fragrance dispenser 100, from the dispenser controller 102, and determine, based on the amount of liquid fragrance in the liquid fragrance cartridge 200, the run time, and the atomization output, a remaining amount of the liquid fragrance in the liquid fragrance cartridge 200. In some embodiments, for example, the cartridge controller 202 is configured to access data from the cartridge memory 216 representative of an amount of the liquid fragrance in the cartridge 200, receive data representative of run time and atomization output of an atomizer, and update in the memory 216, based on the amount of the liquid fragrance in the liquid fragrance cartridge, the run time, and the atomization output, a remaining amount of the liquid fragrance in the liquid fragrance cartridge. In some embodiments, the remaining amount of liquid fragrance in the liquid fragrance cartridge 200 is an updated amount of liquid fragrance in the liquid fragrance cartridge 200 after the atomizer subsystem 172 has been operational for a period of time, such as the run time. In some embodiments, the remaining amount of liquid fragrance in the liquid fragrance cartridge 200 is the amount of liquid fragrance in the liquid fragrance cartridge 200 less the amount of liquid fragrance that is atomized and emitted, which is based on the atomization output and the run time. In some embodiments, the cartridge controller 202 transmits data representative of the remaining amount of liquid fragrance in the liquid fragrance cartridge 200 to the memory 216 to be stored in the memory 216. In some embodiments, for example, the cartridge controller 202 is configured to access data from the cartridge memory 216 representative of an amount of the liquid fragrance in the cartridge 200, receive the data representative of run time and atomization output of the atomizer 174 of the liquid fragrance dispenser 100 from the dispenser controller 102; and update, based on the amount of the liquid fragrance in the cartridge 200, the run time, and the atomization output, a remaining amount of liquid fragrance in the cartridge 200 to the cartridge memory 216. In this regard, the liquid fragrance cartridge 200, via the cartridge controller 202 and the memory 216, has information of its remaining amount of liquid fragrance, such that the liquid fragrance cartridge 200 may be used with other liquid fragrance dispensers 100. In some embodiments, said other liquid fragrance dispensers 100 retrieves the data representative of the liquid fragrance cartridge 200, which, in some embodiments, includes the remaining amount of liquid fragrance in the liquid fragrance cartridge 200 from the liquid fragrance cartridge 200. Accordingly, where the liquid fragrance dispenser 100 and the liquid fragrance cartridge 200 are in operable communication via the short range communication protocol, such as an RFID protocol or an NFC protocol, a liquid fragrance dispenser 100 retrieves the remaining amount of liquid fragrance in the liquid fragrance cartridge 200 from the liquid fragrance cartridge 200 itself, even if a wireless network or data network is unavailable, such that the dispenser controller 102 or the server 300 can automatically determine the remaining amount of liquid fragrance in the liquid fragrance cartridge 200.

In some examples, rather than storing to the cartridge memory 216 the remaining amount of the liquid fragrance in the liquid fragrance cartridge 200, in some example embodiments the cartridge controller 202 is configured to receive and store an amount of the liquid fragrance used (as converted by the cartridge controller 202 or the dispenser controller 102 the run time, and the atomization output). The stored amount of the liquid fragrance used can be accessed later by the cartridge controller 202 or the dispenser controller 102 to calculate the remaining amount of the liquid fragrance in the liquid fragrance cartridge 200.

In some embodiments, for example, the cartridge controller 200 is configured to detect a presence of a liquid fragrance dispenser, and, in response, transmit data representative of the cartridge to the liquid fragrance dispenser.

In some embodiments, the amount of liquid in the liquid fragrance cartridge 200 is a function of the viscosity or the density of the liquid. In some embodiments, the present amount or the updated amount of liquid is the liquid fragrance cartridge 200 is determinable by the liquid fragrance dispensing system 10 without a sensor that is configured to detect an amount of liquid in the liquid fragrance cartridge 200, such as a level sensor or a volume sensor.

In some embodiments, the cartridge controller 202 performs a preliminary processing of the data in the cartridge memory 216, for example, averaging the data for a specific period, and then sending the preprocessed data to the liquid fragrance dispenser 100 via the communication module 203. In some embodiments, the cartridge controller 202 averages the amount of liquid fragrance in the liquid fragrance cartridge 200 over a period of time to determine the rate of consumption of the liquid fragrance stored in the liquid fragrance cartridge 200 by the liquid fragrance dispensing system 10.

The communication module 203 provides communication channels or communication modalities between the cartridge controller 202 of the liquid fragrance cartridge 200 and the liquid fragrance dispenser 100. In some embodiments, the communication module 203 is a wireless communication subsystem. The communication module 203, as depicted in FIG. 14, includes an RF module 208. In some embodiments, the RF ID 201 is included in the RF module 208. The RF module 208 is similar to the RF module 108. The configurations of the RF module 208 is similar to the RF module 108 and Wi-Fi module 110 as described with respect to the liquid fragrance dispenser 100. The Wi-Fi module 110 may be used to communicate with the liquid fragrance dispenser 100, for example, for receiving software updates from the server 300. The RF module 208 of the liquid fragrance cartridge 200 and the RF module 108 of the liquid fragrance dispenser 100 are configured to establish a wireless channel at the frequency band, for example on the unlicensed frequency spectrum, such as 915 MHz. A liquid fragrance cartridge 200 and the liquid fragrance dispenser 100 may communicate with each other via the unlicensed frequency band or channel. In some embodiments, the liquid fragrance cartridge 200 and the liquid fragrance dispenser 100 communicate with each other via a short range communication protocol, such as a radio-frequency identification (RFID) protocol or a near field communication (NFC) protocol. In this regard, the dispenser controller 102 is configured to detect the releasable coupling of the liquid fragrance cartridge 200 to the atomizer subsystem 172. In some embodiments, the dispenser controller 102 is configured to detect that the liquid fragrance cartridge 200

The communication module 203 of the liquid fragrance cartridge 200 and Wi-Fi module 110 of the liquid fragrance dispenser 100 allows the liquid fragrance cartridge 200 to communicate with the liquid fragrance dispenser 100 via a Wi-Fi network. For example, the Wi-Fi network can be used as a backup to the unlicensed frequency spectrum for the cartridge controller 202 of the liquid fragrance cartridge 200 to transmit data from the cartridge memory 216 to the dispenser controller 102 of the liquid fragrance dispenser 100, and receive commands from the dispenser controller 102 of liquid fragrance dispenser 100.

In some embodiments, communication between the liquid fragrance dispenser 100 and the liquid fragrance cartridge 200 is via a short range communication protocol, such as RF ID, Bluetooth™, and Wi-Fi.

The power module 218 supplies power to the liquid fragrance cartridge 200, such as the cartridge controller 202 and the communication module 203. In some embodiments, for example, the power module 218 includes an RF coil that is externally energized. In some embodiments, where the liquid fragrance dispenser 100 and the liquid fragrance cartridge 200 are in operable communication via a short range communication protocol, such as NFC, which is executed by the RF module 108 and the RF module 208, the liquid fragrance cartridge 200 does not have a power module 218. In such embodiments, the liquid fragrance dispenser 100, for example, the RF module 108, is an active component, and the liquid fragrance cartridge 200, for example the RF module 208, is a passive component. In such embodiments, during operation of the liquid fragrance dispenser 100, the liquid fragrance dispenser 100, for example, the communication module 103, generates an electromagnetic or RF field, and the liquid fragrance cartridge 200 can draw its operating power from said electromagnetic or RF field.

In some embodiments, the power module 218 is charged via wireless charging, or charged via electrical communication with a power source, for example, with a metal contact.

In some embodiments, the power module 218 contains one or more disposable batteries, such as CR2032 or AAA batteries. The power module 218 supplies the power to the liquid fragrance cartridge 200 for at least a desired period, such as six months.

In some embodiments, the cartridge controller 202 configures the liquid fragrance cartridge 200 in a deep sleep mode to conserve battery usage while the liquid fragrance cartridge 200 is constantly or periodically transmitting information stored in its memory 216. In some embodiments, the cartridge controller 202 configures the liquid fragrance cartridge 200 to wake up immediately when a predetermined threshold is reached. For example, the liquid fragrance cartridge 200 wakes up upon sensing that information from the cartridge memory 216 is being requested. In some embodiments, the liquid fragrance cartridge 200 wakes up periodically or at specific scheduled time to report the data representative of the liquid fragrance cartridge 200 to the dispenser controller 102 of the liquid fragrance dispenser 100. In some embodiments, the liquid fragrance cartridge 200 reports the remaining battery life to the liquid fragrance dispenser 100, and in response, the liquid fragrance dispenser 100 is configured to report the remaining battery life to the user equipment 400.

In some embodiments, the liquid fragrance cartridge 200 only reports or transmits data to the liquid fragrance dispenser 100, but does not receive commands from the liquid fragrance dispenser 100. In some embodiments, the liquid fragrance cartridge 200 supports bi-directional communication between the liquid fragrance dispenser 100 and the liquid fragrance cartridge 200. In some embodiments, the liquid fragrance cartridge 200 transmits the data from the cartridge memory 216 to the liquid fragrance dispenser 100, and receives commands from the liquid fragrance dispenser 100. In some embodiments, such wireless communications between the liquid fragrance cartridge 200 and the liquid fragrance dispenser 100 use the unlicensed frequency spectrum, and bypass (do not use or require) any short range communication protocol or Wi-Fi Network. In some embodiments, the liquid fragrance dispenser 100 and the liquid fragrance cartridge 200 are in operable communication via a short range communication protocol, such as RFID or NFC, which is executed by the RF module 108 and the RF module 208.

In some embodiments, the server 300 is configured to notify the user, for example, by sending an email to the user equipment 400 if a liquid fragrance cartridge 200 has not transmitted data for a predetermined period.

In some embodiments, the RF modules 108 and 208 are not included in both the liquid fragrance dispenser 100 and the liquid fragrance cartridge 200. In some embodiments, the liquid fragrance dispenser 100 includes more than one status indicator, such as the lighting subsystem 112

In some embodiments, for example, the cartridge controller 202 and the dispenser controller 102 are in operable communication. In some embodiments, for example, the cartridge controller 202 is configured to transmit data representative of the liquid fragrance cartridge 200 to the dispenser controller 102. In some embodiments, for example, the dispenser controller 102 is configured to receive the data representative of the liquid fragrance cartridge 200 transmitted by the cartridge controller 202, and the variably controlling of the output of the liquid fragrance dispenser 100 is based on the data representative of the liquid fragrance cartridge 200.

In some embodiments, to initialize the liquid fragrance dispensing system 10, after the liquid fragrance dispenser 100 is removed from the packaging, the liquid fragrance dispenser 100 is powered on, for example, with an a battery dock or charging dock, a battery of the battery backup 116, or electricity supply from an outlet by plugging the liquid fragrance dispenser 100 into an electrical outlet, for example, on a wall. In some embodiments, the liquid fragrance dispenser 100 is powered on with a user removing a tab that prevents the battery in the liquid fragrance dispenser 100 from powering the liquid fragrance dispenser 100.

After the liquid fragrance dispenser 100 boots up with the electricity supply from a power outlet or a battery, in some embodiments, the liquid fragrance dispenser 100 connects to a cloud service provided by the server 300. As depicted in FIG. 1, in some embodiments, the user may use an electrical user equipment 400, such as a laptop, a smart phone or a tablet, to access the server 300, for example, by a user interface such as a dashboard supported by a web-portal provided by the server 300. In some embodiments, the user equipment 400 open a web browser, such as Internet Explorer® or Chrome®, to register the liquid fragrance dispenser 100 with the server 300 via the interface. In some embodiments, the user registers the liquid fragrance dispenser 100 with the server 300 by providing the user interface a custom code, for example, a 9 digit code, that is assigned to and uniquely identifies the liquid fragrance dispenser 100.

The interface provides a setup sequence to guide a user to complete the registration of the liquid fragrance dispenser 100 with the server 300. In some embodiments, the user provides additional information such as name for the liquid fragrance dispenser 100, location of the liquid fragrance dispenser 100, etc., to the server 300. In some examples, the user provides cell phone numbers for the server 300 to reach the user by text message using, for example, Wi-Fi, and email addresses for the user to receive alerts from the server 300. In some embodiments, the cell phone number and the emails addresses are associated with the relevant people of a premises in which the liquid fragrance dispensing system 20 is deployed, for example, the owner of the premises, friends of owner, landlord of the owner, or the neighbors of the owner. In addition to text messages and emails, the user may access the information provided by the liquid fragrance dispenser 100 from server 300 in various manners, including a website directed to the server 300, mobile applications connected with the server 300, smart home monitoring systems, and the like.

In some embodiments, the liquid fragrance dispenser 100 is controlled at least in part via the user equipment 400, either directly or through the server 300. In some embodiments, the liquid fragrance dispenser 100 is controlled at least in part by the user equipment 400 communicating through via the server 300. In some embodiments, the communications with the liquid fragrance dispenser 100 are through a wireless network communication, such as a wireless Wi-Fi router, located in a building where the liquid fragrance dispenser 100 is located. In some embodiments, the router communicates with the liquid fragrance dispenser 100, the user equipment 400, and is further connected through the Internet to the server 300. In some embodiments, the user equipment 400 is in operable communication with the server though the router, or through another Internet linkage available to it, for example, a cellular telephone network or a Wi-Fi router in another building.

In some embodiments, an application program (“app”) is downloadable and executable in the user equipment 400. The user equipment 400 operates the app that displays information related to operation of the liquid fragrance dispensing system 10 on a screen of the user equipment 400. In some embodiments, a user inputs commands for operation of the liquid fragrance dispensing system 10 through the user equipment 400. In some embodiments, the user equipment 400 stores data related to operation of the liquid fragrance dispensing system 10, which includes user preferences. In some embodiments, data related to operation of the liquid fragrance dispensing system 10 is stored in the server memory 302.

In some embodiments, the server 300 processes instructions between the user equipment 400 and the liquid fragrance dispenser 100. In some embodiments, the server 300 stores customer information related to the operation of the liquid fragrance dispenser 100. In some embodiments, the server 300 receives, processes or stores information from the liquid fragrance dispenser 100 or the liquid fragrance cartridge 200. In some embodiments, the server 300 tracks the number of bursts of fragrance that have been made from the liquid fragrance cartridge 200 so as to provide an estimate of the amount of liquid fragrance in the liquid fragrance cartridge 200 to the user via the user equipment 400. In some embodiments, the server 300 supports a website that enables communication with the user. In some embodiments, the user can download the app from the server 400 through the website. In some embodiments, the user orders replacement liquid fragrance cartridges 200 through the website. In some embodiments, communications between the user and the website occurs through an E-commerce computer that, for example, provides for encrypted or otherwise secure transmission and storage of credit card and other personal information of the user. In some embodiments, the server 300 processes information relevant to the operation of the liquid fragrance dispensing system 10, for example, aggregated information on the purchase or consumption of liquid fragrance cartridges 200 by fragrance type, season, or distribution channel that is used to plan manufacture, shipping, or warehousing of replacement liquid fragrance cartridges 200.

In some embodiments, after being unboxed, the liquid fragrance dispenser 100 is plugged in. Once plugged in, the liquid fragrance dispenser 100 will start up, initialize its Bluetooth radio, Wi-Fi radio and flash memory, and then idle. In some embodiments, the lighting subsystem 112 of the liquid fragrance dispenser 100 will turn red to indicate that there is no Wi-Fi information saved in the flash memory. In some embodiments, the user installs and runs the app onto their user equipment 400.

On launching, the app causes an “add device” wizard to appear on the screen of the user equipment 400. While following the instructions in the wizard, the user will be presented with a screen to input the network information, for example, network name and password, for connecting to a network, such as via the router.

In some embodiments, the app will connect to the liquid fragrance dispenser 100 over a Bluetooth low energy connection (or via Wi-Fi connection initially), and upload the network information to the liquid fragrance dispenser 100. In some embodiments, the liquid fragrance dispenser 100 will then attempt to connect to the router. A success or error response will be returned to the user equipment 400 via Bluetooth or Wi-Fi, and displayed to the user on the screen of the user equipment 400.

Once connected to the router, the liquid fragrance dispenser will attempt to connect to the server 300 over a Message Queue Telemetry Transport (MQTT) connection. In some embodiments, the communication between the liquid fragrance dispenser 100, the server 300, and user equipment 400 uses a pub/sub model in which all parties can subscribe to topics, and post messages to them. All parties listening in on those topics will receive the messages. Once connected to the MQTT server, the liquid fragrance dispenser 100 will remain idle until the user equipment 400 has established a connection to the MQTT server.

Once the wizard is complete on the user equipment 400, a main screen for the app will appear on the user equipment 400. In the background, the app is attempting a connection to the MQTT server. Once connected, the app sends a “status request” packet to a certain channel composed of the MAC address for the liquid fragrance dispenser 100 and the word “control”. The liquid fragrance dispenser 100 (which has subscribed to that channel earlier) will receive this message and report back the presence of the one or more liquid fragrance cartridges 200, as detected by the dispenser controller 102. In some embodiments, other status factors are reported back, such as whether the cover 154 of the liquid fragrance dispenser 100 is closed. In some embodiments, the user interface of the app will show visual cues on the user equipment 400 to indicate that the liquid fragrance cartridges 200 is present or removed from the liquid fragrance dispenser 100.

In some embodiments, the liquid fragrance dispenser remains in a tight loop reporting various parameters, such as changes in the opening or closing of the cover 154, presence of one or more liquid fragrance cartridges 200, detection of the values of the external variables by the sensor subsystem 120 relative to certain reference values, whether the button 156 is being pressed, and whether the capacitive touch surface 158 is being touched. In some embodiments, the dispenser controller 102 or the server 300 listens for messages from the user equipment 400 via the app, such as commands to emit a burst from a liquid fragrance cartridge 200 or from another liquid fragrance cartridge 200.

In some embodiments, when the liquid fragrance dispenser 100 receives a command to emit burst of fragrance, the dispenser controller 102 sends a control command to the atomizer 174 to atomize the liquid in the liquid fragrance cartridge 200, and begins a timer. Once a predetermined amount of time, for example 0.1 to 1.0 seconds, has elapsed, power is disconnected from the atomizer 174 to stop atomizing the liquid fragrance.

In some embodiments, the user, through the user equipment 400 and a user interface of the app, sets schedules for when the user wishes for emission of fragrance from one or more of the liquid fragrance cartridges 200. Once a schedule is entered into the app, the schedule is serialized into a packet and sent over a MQTT channel, via the server 300. The liquid fragrance dispenser 100 will receive the packet, decode it, and store the schedule to its non-volatile flash memory to be executed at the appropriate time.

Since communication occurs over the MQTT server, in some embodiments, the server 300 stores all information in the communications, for example, for analytical and troubleshooting purposes.

In some embodiments, the liquid fragrance dispenser 100 subscribes to a common Over The Air (OTA) update channel. When a firmware update is available, the server 300 will send out a message to all devices listening on that channel that a firmware update is available. The packet will contain a version number, and file name. The liquid fragrance dispenser 100 will compare the version number to its own firmware. If the version number in the message is higher than the current version, the liquid fragrance dispenser 100 will perform a request to download the new firmware file from the server 300. The file will be downloaded via an HTTP GET request. The liquid fragrance dispenser 100 will then validate the firmware using a Cyclic Redundancy Check (CRC). If the CRC passes, the firmware upgrade process will start, and the liquid fragrance dispenser 100 will then reboot.

Next, the user may cause the liquid fragrance cartridge 200 to be recognized by the liquid fragrance dispenser 100, for example by detecting the liquid fragrance cartridge 200 as being in proximity to the liquid fragrance dispenser 100. In some embodiments, once a liquid fragrance cartridge 200 is inserted in the cavity 188 defined by the housing 150 of the liquid fragrance dispenser 100 or placed in a close proximity sufficient for the RF ID reader 104 of the liquid fragrance dispenser 100 to read the identifier contained in the RF ID 201, the identifier associated with the RF ID 201 of the liquid fragrance cartridge 200 is read by the RF ID reader 104 of the liquid fragrance dispenser 100. In some embodiments, the RF ID reader 104 forwards the identifier to the dispenser controller 102 of the liquid fragrance dispenser 100. If the dispenser controller 102 determines that the identifier does not exist in its memory 130 and that the liquid fragrance cartridge 200 associated with the identifier has not recognized or registered with any other liquid fragrance dispensers 100 (e.g. using resident memory 130 and/or verification from the server 300), the dispenser controller 102 stores the identifier of the liquid fragrance cartridge 200 in its memory 130. In some embodiments, the liquid fragrance dispenser 100 sends a dispenser identifier (e.g. hexadecimal identifier, MAC address) to the liquid fragrance cartridge 200, for storing in memory of the liquid fragrance cartridge 200. In some embodiments, after the liquid fragrance cartridge 200 is recognized with the liquid fragrance dispenser 100 and the identifier of the liquid fragrance cartridge 200 has been stored in the memory 130, the liquid fragrance cartridge 200 sends further information to the liquid fragrance dispenser 100, such as the MAC address of the liquid fragrance cartridge 200. As such, the liquid fragrance cartridge 200 is now paired or registered with the liquid fragrance dispenser 100. By making the liquid fragrance cartridge 200 registered in the liquid fragrance dispenser 100, the dispenser controller 102 recognizes the liquid fragrance cartridge 200 by associating the liquid fragrance cartridge 200 with the identifier to uniquely identify the liquid fragrance cartridge 200 in the liquid fragrance dispenser 100. It should be appreciated that in addition to the RF ID near field communications technologies, the liquid fragrance cartridge 200 may send its unique identifier to the liquid fragrance dispenser 100 via any other short range communication protocols, such as infrared or Bluetooth™ or NFC. In some embodiments, the RF ID is a passive RF ID, while in other example embodiments the RF ID is an active RF ID.

If the liquid fragrance cartridge 200 identifier is already associated with previous liquid fragrance dispenser 100, a request can be sent to user equipment 400 associated with the previous liquid fragrance dispenser 100, to query whether the liquid fragrance cartridge 200 can now be registered to the new liquid fragrance dispenser 100. In some embodiments, each of the liquid fragrance cartridges 200 are exclusive in that they can only be paired with one liquid fragrance dispenser 100.

In some embodiments, the liquid fragrance dispenser 100 generates signals to show that the liquid fragrance cartridge 200 has been recognized by the liquid fragrance dispenser 100. For example, the liquid fragrance dispenser 100 generates a beep and flash the lighting subsystem 112 to indicate that the liquid fragrance cartridge 200 has been recognized by and registered with the liquid fragrance dispenser 100, and saved to memory.

After the liquid fragrance cartridge 200 is recognized by the liquid fragrance dispenser 100, the liquid fragrance dispenser 100 sends the information of the liquid fragrance cartridge 200, such as one or more of the identifier contained in the RF ID 201, the manufacturer identification or serial number, vendor identification or serial number, or the MAC address of the liquid fragrance cartridge 200 to the server 300. In some embodiments, the server 300 stores the information of the liquid fragrance cartridge 200 on the memory of the server 300, so that liquid fragrance cartridge 200 is registered with and recognized by, or paired with, the server 300, and the server 300 therefore recognizes the liquid fragrance cartridge 200 and its association (registration) with the liquid fragrance dispenser 100.

After the liquid fragrance cartridge 200 has been registered with the liquid fragrance dispenser 100 and the server 300, all of the subsequent communications between the liquid fragrance cartridge 200 and the liquid fragrance dispenser 100 contain at least one of the sensor identifiers to indicate the source of the data, namely from the liquid fragrance cartridge 200.

Additional liquid fragrance cartridges 200 may be associated to the liquid fragrance dispenser 100 by the same process described above.

After the liquid fragrance cartridge 200 has been registered with the liquid fragrance dispenser 100 and the server 300, in some embodiments, the server 300 initiate a user interface, such as a dashboard, on the user equipment 400. The user equipment 400 communicates with the server 300 using the app, or have a generic application such as a web browser, and use such programs to display the user interface. The information that the liquid fragrance cartridge 200 has registered with the liquid fragrance dispenser 100 is shown on the user interface on the electronic user equipment 400. For example, an icon of a registered or detected liquid fragrance cartridge 200 will appear on the user interface. Dashboard is provided to the electronic user equipment 400 to display the information of system 10.

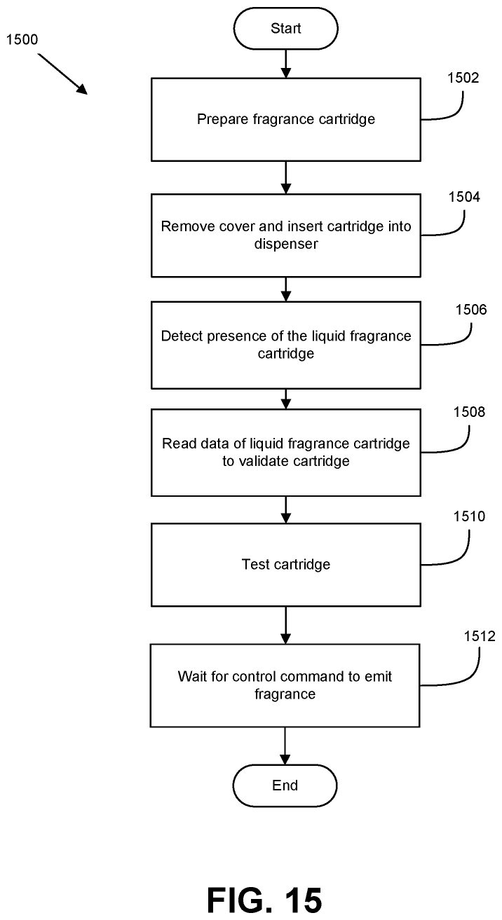

FIG. 15 is a flow chart depicting a method 1500 of loading a liquid fragrance cartridge 200 into a liquid fragrance dispenser 100.

At 1502, the liquid fragrance cartridge 200 is prepared for operable communication with the liquid fragrance dispenser 100. In some embodiments, the liquid fragrance cartridge 200 is filled or refilled, for example, by the manufacturer or a refilling station, and the information representative of the liquid fragrance cartridge 200, such as the unique identification of the cartridge 200, the liquid type contained in the cartridge 200, and the amount of liquid contained in the cartridge 200, is saved onto the memory 216. In some embodiments, one or more caps, seals, and the like, of the liquid fragrance cartridge 200 for preventing or mitigating leakage of liquid fragrance from the cartridge 200 are removed prior to operable communication with the liquid fragrance dispenser 100.

At 1504, the liquid fragrance cartridge 200 and the liquid fragrance dispenser 100 are disposed in operable communication. In some embodiments, the liquid fragrance cartridge 200 is inserted into the cavity 188 defined by the housing 150 of the liquid fragrance dispenser 100.

At 1506, the dispenser controller 102 detects that the cartridge controller 202, and therefore, the liquid fragrance cartridge 200, is present. The dispenser controller 102 confirms that the liquid fragrance dispenser 100 and the liquid fragrance cartridge 200 are in operable communication, for example, by flashing a light via the lighting subsystem 112 or sending a message to the user equipment 400.

At 1508, in some embodiments, the cartridge controller 202 transmits data representative of the liquid fragrance cartridge 200 to the dispenser controller 102. In some embodiments, the data is included in the RF ID 201 of the liquid fragrance cartridge 200, and is read by the RF ID tag reader of the liquid fragrance dispenser 100. The dispenser controller 102 transmits the data to the server 300. The data representative of the liquid fragrance cartridge 200, in some embodiments, includes the unique identifier for the individual liquid fragrance cartridge 200. In some embodiments, the data includes the name or type of the fragrance contained in the liquid fragrance cartridge 200, or the amount of liquid contained in the liquid fragrance cartridge 200. The server 300 processes the data to confirm that it is on a list of valid identifiers (e.g. identifiers applied during the manufacture of liquid fragrance dispensers 200 filled with properly formulated fragrance) before allowing the liquid fragrance dispenser 100 to atomize the liquid fragrance in the liquid fragrance cartridge 200. In some embodiments, the memory 302 contains a database linking the data with information related to the liquid fragrance cartridge 200, such as fragrance type, time of installation, and number of bursts emitted. In some embodiments, where the server 300 does not confirm that the identifier of the liquid fragrance cartridge 200 is on a list of valid identifiers (e.g. the liquid fragrance cartridge 200 is a counterfeit cartridge or improperly reused), the server 300 sends a control command to the dispenser controller 102, such that the dispenser controller 102 sends a control command to the atomizer subsystem 172 to not activate.

At 1510, in some embodiments, the liquid fragrance cartridge 200 is tested by pushing a button rendered on a screen of the user equipment 400 when a liquid fragrance cartridge 200 is installed. In some embodiments, by pushing the test button, the liquid fragrance dispenser 100 atomizes the liquid in the one or more liquid fragrance cartridges 200 to emit a burst of fragrance from the liquid fragrance cartridges 200, and prime the atomizer subsystem 172, for example, by wetting the atomizer disc 176 with the liquid fragrance from the liquid fragrance cartridge 200.

At 1512, the liquid fragrance dispenser 100 waits for a control command, for example, from the server 300, from the user interface subsystem 155 (e.g. the button 156 or the capacitive touch 158), or from the user equipment 400, to emit bursts of fragrance.

In some embodiments, the cartridge controller 202 is in operable communication with the cartridge memory 216, for executing instructions stored in the cartridge memory 216 that, when executed, causes the cartridge controller 202 to transmit data representative of the liquid fragrance cartridge 200. In some embodiments, the dispenser controller 102 is configured to receive the data representative of the liquid fragrance cartridge 200 transmitted by the cartridge controller 202, and based on the data representative of the liquid fragrance cartridge 200, variably control the output of the dispenser 100.

In some embodiments, the dispenser controller 102 is configured to: (i) receive valid cartridge identification information, for example, from the memory 302 or from the memory 130, (ii) receive data representative of a unique identification of the liquid fragrance cartridge 200, (iii) perform a comparison of the valid cartridge identification information and the unique identification of the liquid fragrance cartridge 200, and (iv) determine validity of the cartridge 200 based on the comparison. In other examples, step (iii) is performed using symmetric keys, asymmetric keys, tokens, or other authentication and encryption mechanisms. Data communicated between the dispenser controller 102 and the cartridge controller 202 can be encrypted or signed using symmetric keys or asymmetric keys, as applicable.

In some embodiments, for example, the dispenser controller 102 is configured to receive data representative of a unique identification of the liquid fragrance cartridge 20 and validate the unique identification of the liquid fragrance cartridge 200 from the dispenser memory or through communication with an external device.

In some embodiments, the dispenser controller 102 is configured to, in response to successful validation of the liquid fragrance cartridge 200, activate the atomizer subsystem 172 or the atomizer 174 or vary the atomization output of the atomizer 174.

In some embodiments, the liquid fragrance is contained in the cavity 253 of the liquid fragrance cartridge 200, and the atomizer 174 is disposed in fluid communication with the liquid fragrance. In such embodiments, in response to activation of the atomizer subsystem 172 by the dispenser controller 102 based on the determination of the validity of the liquid fragrance cartridge 200, the liquid fragrance is atomized.

In some embodiments, the dispenser controller 102 is configured to, based on the determination of the validity, vary the output the atomizer subsystem 172.

In some embodiments, the user configures a registered liquid fragrance cartridge 200 on the dashboard displayed on the user equipment 400. In some embodiments, the users assigns a name to a registered liquid fragrance cartridge 200. In some embodiments, the user sets alert parameters of the registered liquid fragrance cartridge 200 on the dash board which is associated with the server 300. The user may choose the desired alerts to receive and the manners of sending the alerts. In some embodiments, the user selects to receive a communication on a user equipment 400 from the server 300, or an email at the email address provided when the amount of liquid fragrance in the liquid fragrance cartridge 200 is below a threshold amount. In some embodiments, the power outage is detected by the dispenser controller 102 from the charging circuit 114, or the liquid fragrance dispenser 100 includes a power sensor, or the power detection circuit, to detect the power supply status for the power outlet, and an alert is sent to the user equipment 400. In some embodiments, these alert parameters are stored on the server 300.

In some examples, the alert parameters are set for one liquid fragrance cartridge 200 at a time or all registered liquid fragrance cartridges 200 at the same time, for example, via the RF modules 108 and 208. In some embodiments, the alert parameters are a threshold of a particular environmental variable defined using the user equipment 400, and an alert is sent to in response to the variable as detected by the sensor subsystem 120 exceeding the threshold (or going below, as appropriate). In some embodiments, a combination of environmental variables are detected by the sensor subsystem 120 and determined as exceeding a combined threshold (or going below, appropriate). In some embodiments, a combined threshold is based on data sensed from two or more sensors of the sensor subsystem 120. In some embodiments, the particular alert parameters are set by a system administrator, the server 300, regulatory or government requirements, or automatically determined using machine learning.

In some embodiments, the user equipment 400 sets operating parameters of a liquid fragrance cartridge 200, such as atomization rate of the atomizer subsystem 172, and reporting said frequency of the atomizer subsystem 172. The user equipment 400 implements the operating parameters by sending them, via the interface of the server 300, to the dispenser controller 102 of the liquid fragrance dispenser 100 for implementing the operating parameters. In some embodiments, the dispenser controller 102 forwards the operating parameters to the cartridge controller 202 of the liquid fragrance cartridge 200 for implementing the operating parameters.

After the one or more liquid fragrance cartridges 200 have been detected and recognized in the liquid fragrance dispenser 100 and configured, a user may select to connect the liquid fragrance dispenser 100 and the liquid fragrance cartridges 200 to a communication network, such as a using RF modules 108 and 208. In some embodiments, a user, at any time, selects to connect the liquid fragrance dispenser 100 to a Wi-Fi network before the liquid cartridges 200 have been detected and recognized in and configured in the liquid fragrance dispenser 100.

In some embodiments, the user selects or clicks on a Wi-Fi configuration button on the user interface, such as a dashboard, displayed on the electronic user equipment 400 to initiate a Wi-Fi network selecting process. In response to the section or click, the server 300 sends a real time text message to the liquid fragrance dispenser 100. The server 300, via the dispenser controller 102, request the Wi-Fi module 110 to scan available Wi-Fi networks and to provide the name of the available Wi-Fi networks. In some embodiments, if the liquid fragrance dispenser 100 is uses a Wi-Fi network when the server 300 sends the real time text message, the liquid fragrance dispenser 100 disconnects the Wi-Fi network in use before scanning available Wi-Fi networks, and then reconnects to the previous Wi-Fi network for sending the scan results to the server 300.

In some embodiments, in response to the user's selection of the Wi-Fi configuration button, the liquid fragrance dispenser 100 generates an audio and/or visual indication to show that the liquid fragrance dispenser 100 starts scanning available Wi-Fi networks in the premises. In some embodiments, the liquid fragrance dispenser 100 generates a beep sound and/or flash the LED 112 to indicate that the liquid fragrance dispenser 100 is scanning for available Wi-Fi network.

In some embodiments, the liquid fragrance dispenser 100 scans available Wi-Fi network and forwarded the information of the Wi-Fi networks to the server 300. The server 300 sends the name of the available Wi-Fi networks to the user equipment 400 on the user interface for the user to select a Wi-Fi network, providing login credentials, such as password, in order for the liquid fragrance dispenser 100 to use the selected Wi-Fi network. For example, the available networks are listed on the dashboard of the electronic user equipment 400. In some embodiments, the user selects the name of an available Wi-Fi network, enters their password, and save the selected Wi-Fi network and the password to the server 300. The liquid fragrance dispenser 100 receives the selected Wi-Fi network and the login credentials from the cloud service provided by the server 300, stores the selected Wi-Fi network and the login credentials in memory 130 of the liquid fragrance dispenser 100. As such, with the information of the selected Wi-Fi network and the login credentials, the liquid fragrance dispenser 100 is connected to the selected Wi-Fi network for subsequent wireless communication. In some example, the lighting subsystem 112 of the liquid fragrance dispenser 100 indicates that the liquid fragrance dispenser 100 has successfully connected to the Wi-Fi network, for example, by flashing with a different color and/or an audible output such as a beep.

In some embodiments, when a Wi-Fi network is not functioning for a period or the liquid fragrance dispenser 100 cannot log into the Wi-Fi network, the server 300 commands the liquid fragrance dispenser 100 to disconnect from the Wi-Fi network by sending a communication, such as “WIFI-CLEAR”.

If a Wi-Fi network becomes unavailable for a predetermined period, in some embodiments, the dispenser controller 102 reports this event to the server 300. The server 300 notifies the user equipment 400, for example, via emails. The server 300 instruct the dispenser controller 102 to remove the Wi-Fi network from the available network list.

In some embodiments, the liquid fragrance cartridge 200 is configured to use a wireless channel for communication, such as unlicensed frequency spectrum, for example, on 915 MHz. A power outage usually accompanies failure of a router, resulting in loss of the Wi-Fi network. In some examples, the unlicensed frequency spectrum is the default setting, and the liquid fragrance cartridge 200 automatically uses a backup wireless modality such as the Wi-Fi network if the unlicensed frequency spectrum is not available.

Referring still to the backup Wi-Fi option of the liquid fragrance cartridge 200, in some embodiments, once the liquid fragrance dispenser 100 has connected to the Wi-Fi network via the Wi-Fi module 108, the dispenser controller 102 of the liquid fragrance dispenser 100 send commands to the liquid fragrance cartridges 200 detected and recognized in the liquid fragrance dispenser 100 for the liquid fragrance cartridge 200 to connect with the Wi-Fi network. The commands may include the name of the selected Wi-Fi network, the login credentials to the Wi-Fi network. Accordingly, with the commands from the liquid fragrance dispenser 100, the liquid fragrance cartridges 200 may be configured to access the same Wi-Fi network with the RF module 208. As well, a respective address such as an IP address (static or dynamic) can be exchanged between the liquid fragrance dispenser 100 and the liquid fragrance cartridge 200, and stored in each other's respective memory. As such, the liquid fragrance dispenser 100 and the liquid fragrance cartridges 200 may communicate with each other by the same Wi-Fi network via respective W-Fi modules.

Other redundancies of wireless modalities can be used in other example embodiments.

In some examples, when Wi-Fi is not available, the other wireless communication modalities are still used as a default. If after a period of time (days), the liquid fragrance dispenser 100 is still unable to connect to a Wi-Fi network, for example, due to a network name change or password change, the electronic user equipment 400 receives an alert and a link from the liquid fragrance dispenser 100. By clicking the link, the liquid fragrance dispenser 100 rescans available Wi-Fi networks at premises. The availability of a Wi-Fi network may not affect the connectivity between the liquid fragrance dispenser 100 and the liquid fragrance cartridge 200. For example, the liquid fragrance cartridge 200 may not need to re-detected and re-recognized in the liquid fragrance dispenser 100 if an available Wi-Fi network becomes unavailable or if no Wi-Fi network is available.

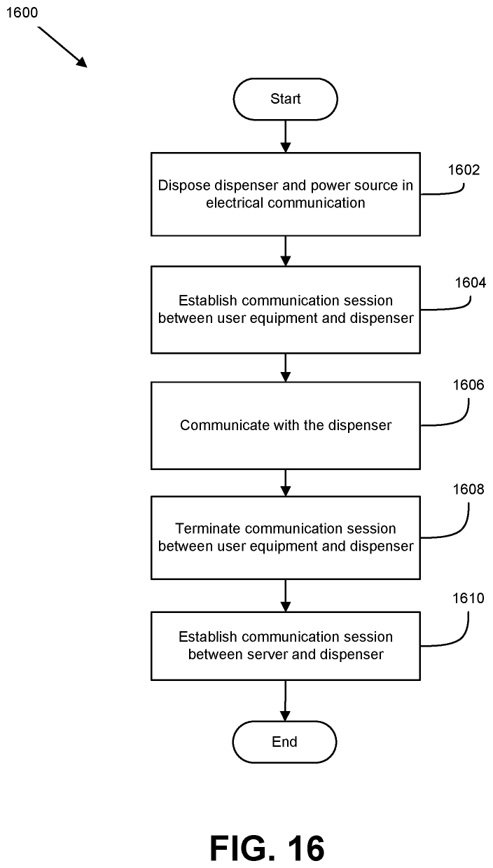

FIG. 16 is a flow chart depicting a method 1600 of initializing the liquid fragrance dispenser 100 and the user equipment 400. In some embodiments, the app is downloaded to the user equipment 400 through the server 300. In some embodiments, the app provides instructions for the remainder of the initial set up process, or these provides may be provide in print or through the server 300 via a website.

At 1602, in some embodiments, the liquid fragrance dispenser 100 is disposed in electrical communication with a power source via the connector 186, such that the power module 107 provides power to the components of the liquid fragrance dispenser 100.

At 1604, a communication session is established between the user equipment 400 and the liquid fragrance dispenser 100, for example, through a router, or directly, for example, by Bluetooth™ communication.

At 1606, the local network name and password are inputted to be used by the server 300 to communicate with the liquid fragrance dispenser 100. In some embodiments, the location of the liquid fragrance dispenser 100 and a customer identifier may be inputted, which, in some embodiments, are generated without reference to any actual personal information of a user. Said customer identifier may be used to help manage multiple liquid fragrance dispensers 100 owned by a single user but placed in different rooms.

At 1608, the communication session between the user equipment 400 and the liquid fragrance dispenser 100 is terminated.

At 1610, the server 300 establishes a communication session with the liquid fragrance dispenser 100. The liquid fragrance dispenser 100 and the server 300 are now in operable communication with each other, for example, through the router.

With the communication network established between the liquid fragrance cartridge 200 and the liquid fragrance dispenser 100, the liquid fragrance cartridge 200 transmits data representative of the liquid fragrance cartridge 200 to the dispenser controller 102. As the liquid fragrance cartridge 200 is associated with a unique sensor identifier contained in the RFID 201 or a MAC address, both registered in the liquid fragrance dispenser 100 and the server 300, the liquid fragrance cartridge 200 sends the data representative of the liquid fragrance cartridge 200 to the liquid fragrance dispenser 100 containing at least the identifier, so that the liquid fragrance dispenser 100 associates received data from the liquid fragrance cartridge 200.

In addition, the sensor subsystem 120 begins to detect values of the external variables and transmit data representative of the values of the detected external variables to the dispenser controller 102. As the liquid fragrance dispenser 100 is associated with a unique identifier, the data representative of the values of the external variables of the environment has at least the identifier, so that the data is associated with the liquid fragrance dispenser 100.

In some embodiments, the liquid fragrance dispenser 100 sends the data from the liquid fragrance cartridge 200 and from the sensor subsystem 120 to the server 300, via the Wi-Fi module 110. In some embodiments, the Wi-Fi is the default and another communication modality is the backup.

As described above, the liquid fragrance dispenser 100 is registered with server 300 by providing a unique liquid fragrance dispenser identifier, such as custom code, or hexadecimal number. In some embodiments, the liquid fragrance dispenser 100 sends the data from the sensor subsystem 120 and the liquid fragrance cartridge 200 to the server 300 by adding its custom code in the sensed data. As such, the server 300 knows the source of the data, such as from a specific liquid fragrance cartridge 200 and a specific liquid fragrance dispenser 100. In some embodiments, the dispenser controller 102 preliminarily processes the data from the sensor subsystem 120 and from the liquid fragrance cartridge 200, and only sends the processed results to the server 300, together with the identifier of the liquid fragrance cartridge 200 and the custom code of the liquid fragrance dispenser 100.

In some embodiments, the server 300 then processes the data received from the liquid fragrance dispenser 100, such as the data representative of the value of the external variable of the environment of the liquid fragrance dispensing system 10, and the data representative of the liquid fragrance cartridge 200. In some embodiments, the server 300 compares the data with the alert parameters set by the user to determine whether an alert should be sent to the user equipment 400. In some embodiments, the sensor subsystem 120 detects the ambient temperature, the ambient humidity, operating conditions of the liquid fragrance dispenser 100, acceleration of the liquid fragrance dispenser 100, occupancy detection, and ambient light intensity. In some embodiments, the data representative of the liquid fragrance cartridge 200 includes fluid type, fragrance strength or concentration, viscosity, date of manufacturing, expiry date, a unique identification of the cartridge, and the amount of liquid fragrance.

In some embodiments, all of the communication from the server 300 to the electronic user equipment 400 indicates the name or identifier of the liquid fragrance dispenser 100, the name or the identifier of the liquid fragrance cartridge 200, and the type of error condition. For example, in some embodiments, the communications note that the actual environmental condition of a particular liquid fragrance dispenser 100 is different (e.g. higher or lower) than the target environmental fragrance condition. In some embodiments, the communications note that a particular liquid fragrance cartridge 200 contains an amount of liquid below a threshold amount. This information allows a user to quickly and readily recognize a condition and its associated locations, especially when a user has multiple liquid fragrance dispensers 100 at different premises and multiple liquid fragrance cartridges 200 per liquid fragrance dispenser 100.

In some embodiments, the server 300 sends an alert to the electronic user equipment 400 when the alert conditions defined in the user equipment 400 are triggered. For example, an alert is triggered when the actual environmental condition, as determined by the dispenser controller 102 based on the sensed data from the sensor subsystem 120, is different from the target environmental fragrance condition as set by the user by a particular range, or when the amount of liquid fragrance in the cartridge 200 is below a threshold amount. For example, when an alert is triggered in server 300, the server 300 sends the alert to the electronic user equipment 400, such as by a communication using Wi-Fi, or by an email. In some embodiments, the server 300 also changes in status colour in the user interface, such as on the dashboard, or pop up windows or messages on the user interface displayed on the electronic user equipment 400. When the actual environmental condition is within an acceptable range of the target environmental fragrance condition, or when the amount of liquid fragrance in the liquid fragrance cartridge 200 is above a threshold amount, the server 300 sends another alert to the electronic user equipment 400 to in the same manners.

In some embodiments, the liquid fragrance dispenser 100 processes the data relating to the values of the external variables and determines the alert conditions, and notifies the server 300, who in turn notifies the user equipment 400. In some embodiments, each liquid fragrance cartridge 200 is configured to process its own sensed environmental variables and is configured to determine the alert conditions, and notifies the liquid fragrance dispenser 100, who in turn notifies the server 300, who in turn notifies the user equipment 400.

In some examples, the server 300 instructs a liquid fragrance dispenser 100 or a liquid fragrance cartridge 200 to reboot, for example, by sending a command in a communication via Wi-Fi from the server 300 to the liquid fragrance dispenser 100. When the liquid fragrance dispenser 100 receives the command for the server 300 to reboot, the dispenser controller 102 of the liquid fragrance dispenser 100 performs the reboot. If the command is for a liquid fragrance cartridge 200 registered under the liquid fragrance dispenser 100 to reboot, the dispenser controller 102 forwards this command to the cartridge controller 202 of the liquid fragrance cartridge 200 to reboot.

In some embodiments, the liquid fragrance dispensing system 10 is configured to variably control an output of the liquid fragrance dispenser 100 based on the data representative of the liquid fragrance cartridge 200.

In some embodiments, the cartridge controller 202 is configured to transmit data representative of a concentration of a solute in the liquid fragrance cartridge 200 (e.g. a strength of the liquid fragrance) and a viscosity of the liquid fragrance contained in the liquid fragrance cartridge 200 to the dispenser controller 120. The dispenser controller 102 is configured to determine a target environmental fragrance condition, and determine an output from the atomizer subsystem 172 (e.g. duration of operation, atomization output of the atomizer subsystem 172). The dispenser controller 102 is further configured to receive, from the cartridge controller 202, the data representative of the concentration of the solute in the liquid fragrance and the viscosity of the liquid fragrance, and determine an actual environmental condition based on the data representative of the atomization output of the atomizer 172, the concentration of the solute in the liquid fragrance, and the viscosity of the liquid fragrance. The dispenser controller 102 is configured to variably control the output of the dispenser 100 by being configured to variably control the duration of operation or atomization output of the atomizer to achieve the target environmental fragrance condition based on the actual environmental condition. In some embodiments, for example, the dispenser controller 102 is configured to determine an actual environmental condition based on the data representative of the atomization output of the atomizer subsystem 172, the concentration of the solute in the liquid fragrance, and the viscosity of the liquid fragrance, determine the operating configuration of the atomizer subsystem 172 to achieve the target environmental fragrance condition based on the actual environmental condition, and variably control the output of the atomizer subsystem 172 based on the determination.

In this regard, the output of the atomizer subsystem 172 can be variably controlled based on the data representative of the liquid fragrance cartridge 200.

In some embodiments, the actual environmental condition is determined by the dispenser controller 102 based on the data representative of the liquid dispenser cartridge 200 and the data representative of the value of the external variable of the environment of the liquid dispensing system 10.

In some embodiments, the dispenser controller 102 is configured to receive, from the sensor subsystem 120, data representative of the external variable detected by the sensor subsystem 120, determine the actual environmental condition based on the data representative of the atomization output of the atomizer subsystem, the concentration of the solute in the liquid fragrance, the viscosity of the liquid fragrance, and the external variable, and to variably control the output of the dispenser 100 by being configured to variably control the duration of operation or atomization output of the atomizer subsystem 172 to achieve the target environmental fragrance condition based on the actual environmental condition. In this regard, the determining of the actual environmental condition is based on a characteristic of the liquid fragrance cartridge 200 and on the value of the external variable, and the output of the atomizer subsystem 172 can be variably controlled based on the data representative of the liquid fragrance cartridge 200, and the data detected by the sensor subsystem 120.

In some embodiments, the server 300 is configured to variably control the output of the atomizer subsystem 172 based on the data representative of the liquid fragrance cartridge 200 and the data detected by the sensor subsystem 120. In such embodiments, the cartridge controller 202 is configured to transmit data representative of a concentration of a solute in the liquid fragrance cartridge 200 (e.g. a strength of the liquid fragrance) and a viscosity of the liquid fragrance contained in the liquid fragrance cartridge 200 to the dispenser controller 120. The dispenser controller 102 is configured to determine a target environmental fragrance condition, determine an amount of atomization output of the atomizer, and receive, from the cartridge controller 202, the data representative of the concentration of the solute in the liquid fragrance and the viscosity of the liquid fragrance. The dispenser controller 102 is configured to transmit said information and data to the server 300, and the server 300 is configured to determine an actual environmental condition based on the data representative of the atomization output of the atomizer subsystem 172, the concentration of the solute in the liquid fragrance, and the viscosity of the liquid fragrance, and send a control command to the dispenser controller 102 to variably control the output of the dispenser 100, for example, by variably controlling the duration of operation or the atomization output of the atomizer subsystem 172 to achieve the target environmental fragrance condition based on the actual environmental condition.

In some embodiments, the dispenser controller 102 is configured to receive, from the sensor subsystem, data representative of the value of the external variable detected by the sensor subsystem 102, and transmit the information and data to the server 300, which is configured to determine the actual environmental condition based on the data representative of the atomization output of the atomizer subsystem 172, the concentration of the solute in the liquid fragrance, the viscosity of the liquid fragrance, and the value of the external variable, and to variably control the output of the dispenser 100 by being configured to variably control the duration of operation or atomization output of the atomizer 174 to achieve the target environmental fragrance condition based on the actual environmental condition.

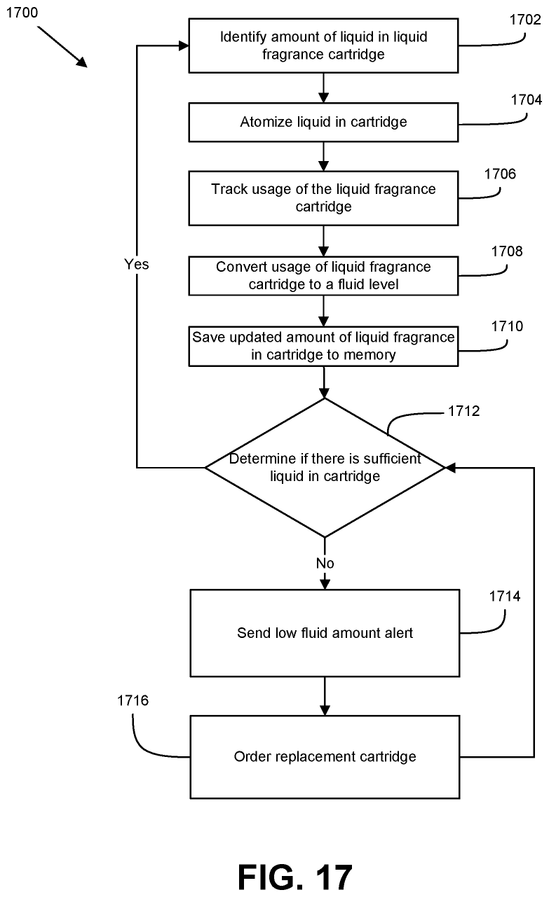

FIG. 17 is a flow chart depicting a method 1700 of managing use of a liquid fragrance cartridge 200.

At 1702, after a new liquid fragrance cartridge is in operable communication with the liquid fragrance dispenser 100, as described herein, the present amount of liquid in the cartridge 200 is determined, for example, from data transmitted by the cartridge 200 via the cartridge controller 202 saved on the memory 216, or from data transmitted by the server 300 from a database maintained by the server 300, or from data transmitted by the dispenser controller 102 from the memory 130. In some embodiments, data representative of the amount of liquid fragrance that is presently in the cartridge 200 is stored in the memory 216, and the cartridge controller 202 is configured to transmit the data to the server 300, or transmit the data to the dispenser controller 102, which then transmits the data to the server 300 for the server 300 to determine the amount of liquid fragrance in the liquid fragrance cartridge 200. In some embodiments, the liquid fragrance dispenser 100, in particular the dispenser controller 102, is configured to determine the amount of liquid fragrance in the liquid fragrance cartridge 200.

At 1704, the dispenser controller 102 sends a control command to the atomizer subsystem 172 to atomize the liquid in the liquid fragrance cartridge 200. In some embodiments, the atomization output of the atomizer subsystem 172 is based on at least one of, or both of, an external variable of the environment of the liquid fragrance dispensing system 10 that is detected by the sensor subsystem 120, and data representative of the liquid fragrance cartridge 200.

At 1706, in some embodiments, as the liquid fragrance dispenser 100 is operational and the atomizer subsystem 172 is atomizing the liquid fragrance, the usage of the liquid fragrance cartridge 200, or the amount of emitted liquid fragrance from the liquid fragrance cartridge 200 is tracked. For example, the number of bursts of liquid fragrance is counted. To track the usage of the liquid fragrance cartridge 200 or the emissions from the liquid fragrance dispenser 100, in some embodiments, the liquid fragrance dispenser 100 sends a message to the server 300 every time the atomizer subsystem 172 is activated to emit a burst of fragrance, the server 300 tracks instructions given to the liquid fragrance dispenser 100 through the user equipment 400 or a burst button 156 on the liquid fragrance dispenser 100, the dispenser controller 102 or the server 300 tracks the periods of times that the atomizer subsystem 172 is active and the atomization output of the atomizer subsystem 172 at said times, or the liquid fragrance dispenser 100 maintains a count in its memory 130. In some embodiments, the count is maintained either as a forward count starting from 0 to a predetermined maximum number of bursts that can be emitted from a liquid fragrance cartridge 200, or as a count down starting from the predetermined maximum number of bursts that can be emitted from a liquid fragrance cartridge 200.

At 1708, the usage of the liquid fragrance cartridge 200 or the amount of emitted liquid fragrance is converted to an estimated amount of liquid fragrance in the liquid fragrance cartridge 200. For example, the burst count corresponds to an amount of liquid fragrance dispersed. In some embodiments, this conversion is done automatically as the count is updated. The conversion is expressed, for example, as a percentage of liquid fragrance used or remaining, or the amount of liquid fragrance dispensed as a mass or a volume, on the user equipment 400 while the user has the app open, or as an alert sent from time to time from the server 300 or the liquid fragrance dispenser 100 to the user equipment 400. In some embodiments, data representative of the amount of liquid remaining in the liquid fragrance cartridge 200 is transmitted to the cartridge controller 202 to be stored in the memory 216. In some embodiments, the amount of emitted liquid fragrance is a function of the run time of the atomizer subsystem 172 and the atomization rate or output of the atomizer subsystem 172. In some embodiments, the remaining amount of liquid fragrance in the liquid fragrance cartridge 200 is the initial amount of liquid fragrance less the emitted or dispersed amount of liquid fragrance.

At 1710, In some embodiments, for example, the dispenser controller 102 is configured to determine the remaining amount of liquid in the liquid fragrance cartridge 200 by receiving the data representative of the present amount of liquid in the liquid fragrance cartridge 200 and the run time and atomization output of the atomizer subsystem 172, determines the amount of liquid that was dispersed based on tracked usage of the liquid fragrance cartridge, which, in some embodiments, is based on the amount of bursts, the amount of emitted fragrance in each burst, and the run time of the atomizer subsystem 172, and determines the remaining amount of liquid in the liquid fragrance cartridge 200 by taking the difference between the present amount of liquid in the liquid fragrance cartridge 200 and the amount of dispersed liquid. In some embodiments, for example the remaining amount of liquid in the liquid fragrance cartridge 200 is saved to the memory 130. In some embodiments, the dispenser controller 102 is configured to transmit data representative of remaining amount of liquid in the liquid fragrance cartridge 200 to the server 300.

In some embodiments, for example, the cartridge controller 202 is configured to determine the remaining amount of liquid in the liquid fragrance cartridge 200 by receiving the data representative of the present amount of liquid in the liquid fragrance cartridge 200 and the run time and atomization output of the atomizer subsystem 172. In some embodiments, for example, the cartridge controller 202 transmits data representative of the remaining amount of liquid in the liquid fragrance cartridge 200 to the memory 216 to be stored in the memory 216. In this regard, the liquid fragrance cartridge 200 is usable with other liquid fragrance dispensers 100, as the remaining amount of liquid in the liquid fragrance cartridge 200 is saved in the memory 216.

In some embodiments, for example, the server 300 is configured to determine the remaining amount of liquid in the liquid fragrance cartridge 200 by receiving the data representative of the present amount of liquid in the liquid fragrance cartridge 200 and the run time and atomization output of the atomizer subsystem 172. In some embodiments, for example the remaining amount of liquid in the liquid fragrance cartridge 200 is saved to the memory 302. In some embodiments, the server 300 is configured to transmit data representative of remaining amount of liquid in the liquid fragrance cartridge 200 to the dispenser controller 102.

At 1712, the dispenser controller 102 or the server 300 determines if there is sufficient liquid in the liquid fragrance cartridge 200. If the dispenser controller 102 or the server 300 determines that there is sufficient liquid in the liquid fragrance cartridge 200, then the amount of liquid in the cartridge 200 is identified in accordance with 1702. If the dispenser controller 102 or the server 300 determines that there is insufficient liquid in the liquid fragrance cartridge 200, then a low fluid amount alert is sent in accordance with 1708.

When the dispenser controller 102 or the server 300 determines that a threshold amount of liquid fragrance has been reached or that a threshold amount of fragrance has been dispensed for a particular liquid fragrance cartridge 200, it deems the liquid fragrance cartridge 200 to be sufficiently low in liquid fragrance such that its replacement or replenishment should be planned to enable continued use.

In some embodiments, to indicate that one or more liquid fragrance cartridges 200 is running low on liquid fragrance, the dispenser controller 102 or the server 300 sends a control command to the user interface subsystem 155 of the liquid fragrance dispenser 100, such as the speaker 124 or the lighting subsystem 112, to generate a sound or emit a light, such as one or more indicator lights, lighting patterns or colors, sounds, vibration, etc. The dispenser controller 102 or the server 300 may also be configured to push a message via email, Wi-Fi, etc. to the user equipment 400.

In some embodiments, an NFC tag is attached to the liquid fragrance cartridge 200 and is programmed with information describing the liquid fragrance cartridge 200, for example, a maximum number of bursts of the liquid fragrance cartridge 200, and an encrypted serial number. When the liquid fragrance cartridge 200 is disposed in operative communication with the liquid fragrance dispenser 100, the dispenser controller 102 polls the NFC tag, receives and decrypts the serial number, and sends the serial number to the server 300 for verification. If the serial number is recognized by the server 300 as representing a valid or verified liquid fragrance cartridge 200, the server 300 instructs the liquid fragrance dispenser 100 to enter a bursting mode. In the bursting mode, the liquid fragrance dispenser 100 will act on instructions to emit bursts of fragrance. In some embodiments, the liquid fragrance dispenser 100 maintains a counter within its flash memory 130 to track the number of bursts emitted from the liquid fragrance cartridge 200. Once the number of bursts is reached, the liquid fragrance dispenser 100 transitions itself to a non-bursting mode on and will no longer emit bursts from liquid fragrance cartridge 200.

Once the amount of liquid fragrance in the liquid fragrance cartridge 200 is deemed to be below a threshold level, for example, once the maximum number of bursts has been reached, the server 300 will cancel all future instructions to emit bursts, or instruct the liquid fragrance dispenser 100 to enter a non-bursting mode, or the liquid fragrance dispenser 100 will put itself into a non-bursting mode. This prevents further bursts from being emitted from the liquid fragrance cartridge 200 linked to the count. The liquid fragrance cartridge 200 is discarded. Further bursts are permitted only after a unique identifier from a new replacement liquid fragrance cartridge 200 is provided as the new liquid fragrance cartridge 200 is loaded. The server 300 will only validate a liquid fragrance cartridge 200 once, such that a liquid fragrance cartridge 200 cannot be used after it is deemed empty.

At 1714, once the amount of liquid fragrance in the liquid fragrance cartridge 200 is deemed to be below a threshold level, for example, once the number of bursts remaining reaches a predetermined level, the server 300 or the dispenser controller 102 sends a low fragrance amount alert to the user equipment 400. The alert, once received by the user equipment 400 and processed by the app, provides a notice on the screen of the user equipment 400 indicating that the amount of fragrance in the liquid fragrance cartridge 200 is low. In some embodiments, the alert message is displayed continuously or periodically while the liquid fragrance dispenser 100 continues to emit bursts from the liquid fragrance cartridge 200. In some embodiments, in response to the alert, the dispenser controller 102 sends a control command to the user interface subsystem 155 to, for example, emit a light or light pattern with the lighting subsystem 112, such as a flashing or continuous yellow or red colour, or to emit a sound with the speaker 124.

At 1716, in response to the alert, the server 300 or the dispenser controller 102 sends a control command to the user equipment 400, such that a virtual button is displayed on the screen of the user equipment 400 via the app. A control command can be transmitted to the server 300 from the user equipment 400 representative of ordering a replacement liquid fragrance cartridge 200. The server 300 processes the ordering message according to a replacement cartridge protocol.

In some embodiments, after the new cartridge 200 is ordered and disposed in operable communication with the liquid fragrance dispenser 100, the dispenser controller 102 or the server 300 determines if there is sufficient liquid in the new liquid fragrance cartridge 200, in accordance with 1707.

In some embodiments, the user can shop for one or more liquid fragrance cartridge 200 from the website, which may contain a larger selection of fragrances, payment, or delivery options.

In some embodiments, the cartridge controller 202 is configured to track usage of the liquid fragrance cartridge 200, and configured to convert the usage of the liquid fragrance cartridge 200 to a fluid level, an amount of remaining liquid fragrance, a percentage of remaining liquid fragrance, and the like.

In some embodiments, the method 1700 is performed continuously in real time. In some embodiments, the method 1700 is performed periodically, for example, every one minute. In some embodiments, a portion of the method 1700, such as 1702 and 1704, is performed for a given fragrance session, and the remaining portion of the method 1700 is performed upon completion of the fragrance session.

In some embodiments, when the dispenser controller 102 or the server 300 determines that a threshold value has been reached for the amount of liquid fragrance in the liquid fragrance cartridge 200 and deems that a replacement or replenishment is needed, the dispenser controller 102 or the server 300 transmits a notification to the application executing on user equipment 400. The application on the user equipment, when executed, polls the dispenser controller 102 over the wireless network to determine the status information of the liquid fragrance dispenser 100. This status information includes its current configuration, and whether the liquid fragrance in one or more of the liquid fragrance cartridge 200 is running low and needs replenishing. If the received status information indicates that a liquid fragrance cartridge 200 should be replaced, the application executing on the user equipment 400 presents an alert screen to the user. The alert screen presents a button asking the user if they would like to order a refill liquid fragrance cartridge 200. Upon activation of the button for the first time by a user, the application presents another screen wherein the user can make a liquid fragrance and quantity selection, and will then be prompted for shipping and billing information. Upon completing the second screen and activating an order button after confirming the order, the order is processed. For subsequent refills, the user may choose to use the information previously provided or may edit the information.

In some embodiments, the liquid fragrance dispensing system 10 is configured to automatically order a replacement liquid fragrance cartridge 200 upon determination by the dispenser controller 102 or the server 300 that the amount of liquid fragrance in the liquid fragrance cartridge 200 being used to dispense the liquid fragrance is below a threshold amount. In some embodiments, when it is determined that the amount of liquid fragrance in the liquid fragrance cartridge 200 is below a threshold amount, the dispenser controller 102 or the server 300 automatically orders a replacement liquid fragrance cartridge 200. In some embodiments, the data representative of the replacement liquid fragrance cartridge 200 that is ordered is the same as the data representative of the liquid fragrance cartridge 200 that is being used. In some embodiments, the replacement liquid fragrance cartridge 200 that is ordered is based on the user's selection of liquid fragrance and quantity that was input into the user equipment 400 when first ordering a replacement liquid fragrance cartridge 200.

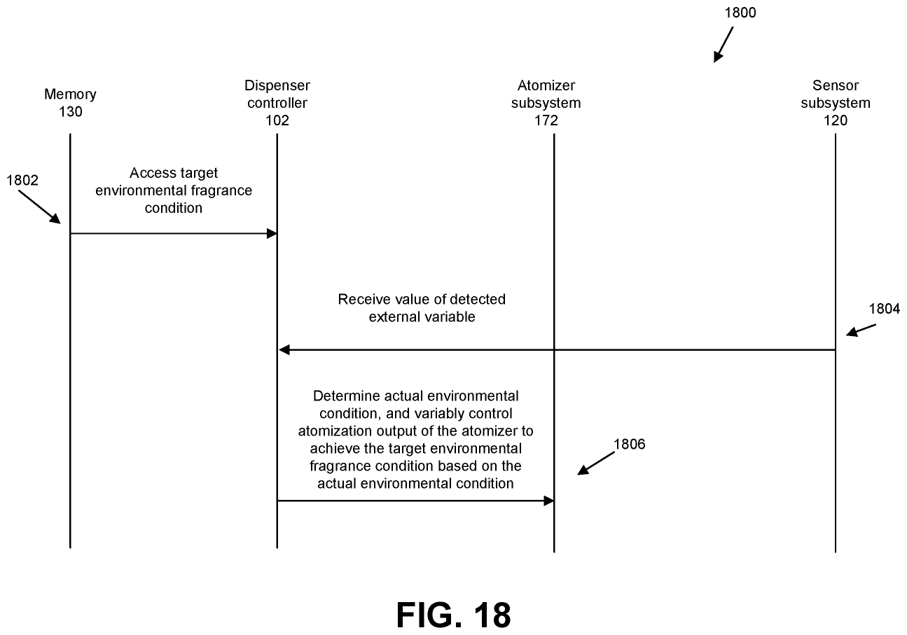

FIG. 18 is a flow chart depicting a method 1800 of variably controlling an output of the liquid fragrance dispenser.

At 1802, the dispenser controller 102 accesses data representative of a target environmental fragrance condition. In some embodiments, for example, the dispenser controller 102 reads the memory 130 to access data representative of a target environmental fragrance condition to determine the target environmental fragrance condition.

At 1804, the dispenser controller 102 receives data representative of the value of the external variable detected by the sensor subsystem 120.

At 1806, the dispenser controller 102 determines an actual environmental condition based on the data representative of the atomization output of the atomizer subsystem 172 and the value of the external variable detected by the sensor subsystem 102, and determines the output of the atomizer subsystem 172 to achieve the target environmental fragrance condition. Based on this determination, the dispenser controller 102 variably controls the output of the atomizer subsystem 172, for example, the duration of operation or atomization output of the atomizer subsystem 172, to achieve the target environmental fragrance condition based on the actual environmental condition. In some embodiments, the target environmental fragrance condition is achieved further based on previous atomization output of the atomizer.

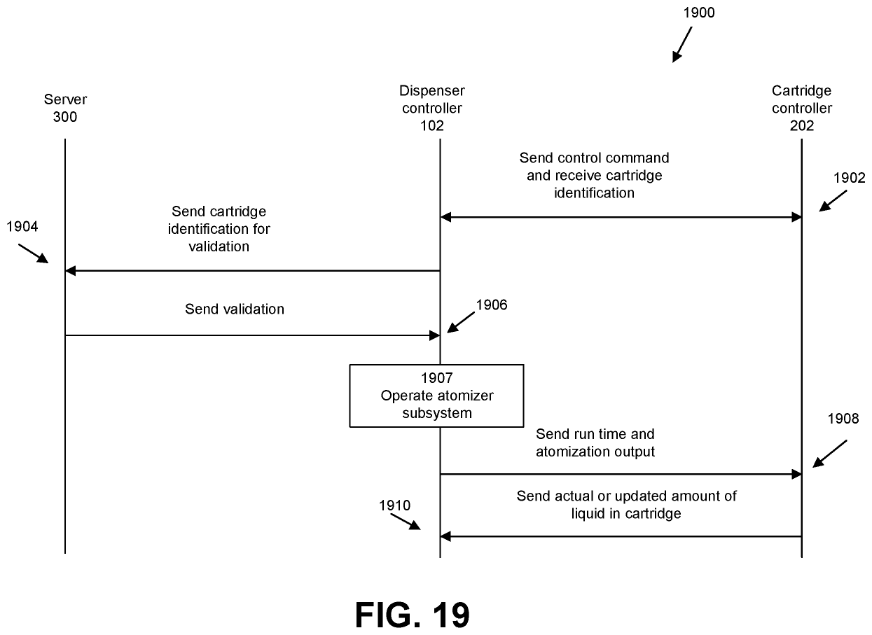

FIG. 19 is a flow chart depicting a method 1900 of determining an amount of liquid fragrance in a liquid fragrance cartridge.

At 1902, while the liquid fragrance cartridge 200 and the liquid fragrance dispenser 100 are in operable communication, the dispenser controller 102 sends a control command to the cartridge controller 202 to transmit data representative of a cartridge identification, and in response, the dispenser controller 102 receives the cartridge identification from the cartridge controller 202.

At 1904, the dispenser controller 102 sends the cartridge identification to the server 300 to validate the liquid fragrance cartridge 200. In some embodiments, the server 300 compares the cartridge identification with a list of cartridge identifications stored in the memory 302 to validate the liquid fragrance cartridge 200.

At 1906, upon validation of the liquid fragrance cartridge 200, the server 300 sends a validation to the dispenser controller 102 to indicate to the dispenser controller 102 that the liquid fragrance cartridge 200 is a valid cartridge (e.g. the cartridge 200 is manufactured from an approved manufacturer and is not a counterfeit cartridge 200).

At 1907, the liquid fragrance dispenser 100 is activated. In some embodiments, for example, the dispenser controller 102 sends a control command to the atomizer subsystem 172 to operate for a run time at an atomization output.

At 1908, upon validation of the liquid fragrance cartridge 200, the dispenser controller 102 transmits data representative of the run time and the atomization output of the atomizer subsystem 172 to the cartridge controller 202.

In some embodiments, the cartridge controller 202 determines a remaining amount of liquid in the liquid fragrance cartridge 200. The cartridge controller 202 sends a control command to the memory 216 for the memory 216 to transmit data to the cartridge controller 202 representative of the present amount of liquid in the liquid fragrance cartridge 200, determines the amount of liquid that was dispersed based on the run time and atomization output of the atomizer subsystem 172, and determines the remaining amount of liquid in the liquid fragrance cartridge 200 by taking the difference between the present amount of liquid in the liquid fragrance cartridge 200 and the amount of dispersed liquid. In some embodiments, the cartridge controller 202 transmits data representative of the remaining amount of liquid in the liquid fragrance cartridge 200 to the memory 216 to be stored in the memory 216.

In some embodiments, the server 300 is configured to determine the remaining amount of liquid in the liquid fragrance cartridge 200 by receiving the data representative of the present amount of liquid in the liquid fragrance cartridge 200 and the run time and atomization output of the atomizer subsystem 172. In some embodiments, for example the remaining amount of liquid in the liquid fragrance cartridge 200 is saved to the memory 302. In some embodiments, the server 300 is configured to transmit data representative of remaining amount of liquid in the liquid fragrance cartridge 200 to the dispenser controller 102.

In some embodiments, the dispenser controller 102 is configured to determine the remaining amount of liquid in the liquid fragrance cartridge 200 by receiving the data representative of the present amount of liquid in the liquid fragrance cartridge 200 and the run time and atomization output of the atomizer subsystem 172. In some embodiments, for example the remaining amount of liquid in the liquid fragrance cartridge 200 is saved to the memory 130. In some embodiments, the dispenser controller 102 is configured to transmit data representative of remaining amount of liquid in the liquid fragrance cartridge 200 to the server 300.

At 1910, the dispenser controller 102 sends a control command to the cartridge controller 202, and in response, the cartridge controller 202 sends data representative of the remaining amount of liquid in the liquid fragrance cartridge 200 to the dispenser controller 102. In some embodiments, the dispenser controller 102 sends the data representative of the remaining amount of liquid in the liquid fragrance cartridge 200 to the server 300. In this regard, the liquid fragrance cartridge 200 is configured to be in operable communication with, and be compatible to be used with, other liquid fragrance dispensers 100.

In some embodiments, as depicted in FIG. 12, the liquid fragrance dispensing system 10 includes more than one liquid fragrance cartridge 200. In such embodiments, each of the liquid fragrance cartridges 200 are independently controllable by the dispenser controller 102 or the server 300, and that there is an atomizer 174 for each one of the liquid fragrance cartridges 200. In this regard, the liquid fragrance dispensing system 10 is configured to variably control an output of the liquid fragrance dispenser 100 by independently controlling the output of each of the atomizers 174, and thus, independently controlling the dispersion of the liquid fragrance contained in the liquid fragrance cartridges 200, such that the liquid fragrance dispensing system 10 can mix the fragrances contained in the liquid fragrance cartridges 200 to define the environmental condition, and the user has a choice of scents to disperse.

In some embodiments, where the liquid fragrance dispensing system 10 includes more than one liquid fragrance cartridge 200, for example, two liquid fragrance cartridges 200, the RF module 108 of the liquid fragrance dispenser 100 includes one antenna for each one of the liquid fragrance cartridges 200, such that data communication between the dispenser controller 102 and the cartridge controller 202 of each of the plurality of liquid fragrance cartridges 200 may be achieved, independently, via the corresponding antenna of the plurality of antennas of the RF module 108. For example, where there are two liquid fragrance cartridges 200, the RF module 108 includes two antennas. The first antenna is for communication between the dispenser controller 102 and the cartridge controller 202 of the first liquid fragrance cartridge 200, and the second antenna is for communication between the dispenser controller 102 and the cartridge controller 202 of the second liquid fragrance cartridge 200. In this regard, for example, the first fragrance cartridge 200 is identified by the dispenser controller 102 via the first antenna, and the second fragrance cartridge 200 is identified by the dispenser controller 102 via the second antenna. In some embodiments, the first antenna is disposed relatively proximate to a first cavity 188 and relatively farther from a second cavity 188, and the second antenna is disposed relatively farther from the first cavity 188 and relatively proximate to the second cavity 188, such that the cavity 188 in which a liquid fragrance cartridge 200 is inserted is identifiable by the dispenser controller 102 based on the data communication via the first or second antenna.

In some embodiments, the liquid fragrance dispensing system 10 includes more than one liquid fragrance dispenser 100. In such embodiments, each of the liquid fragrance dispensers 100 are operable together. In some embodiments, the schedule of operation that is input by the user via the user equipment 400, or that is learned by the liquid fragrance dispensing system 10 via, for example, machine learning, is transmitted by the server 300 to each of the liquid fragrance dispensers 100, such that the liquid fragrance dispensers 100 operate based on a single schedule of operation. In some embodiments, the operating parameters that are input by the user via the user equipment 400, or that are learned by the liquid fragrance dispensing system 10 via, for example, machine learning, such as a target environmental fragrance condition, are transmitted by the server 300 to each of the liquid fragrance dispensers 100. In this regard, each of the liquid fragrance dispensers 100 can detect the value of the external variable on an environment of the liquid fragrance dispensing system 10 and variably control an output of the liquid fragrance dispenser 100, for example, control the duration of operation or atomization output of the respective atomizer subsystems 172, based on the same operating parameters, such as the same target environmental fragrance condition.

In some embodiments, the user, via the user equipment 400, can send a control command to the server 300 to synchronize the liquid fragrance dispensers 100 in the liquid fragrance dispensing system 10.

Through the descriptions of the example embodiments, the example embodiments may be implemented by using hardware only or by using software and a necessary universal hardware platform. Based on such understandings, the technical solution of some example embodiments may be embodied in the form of a software product. The software product may be stored in a non-volatile or non-transitory storage medium, which can be a compact disk read-only memory (CD-ROM), USB flash disk, or a removable hard disk. The software product includes a number of instructions that enable a computer device (personal computer, server, or network device) to execute the methods provided in the example embodiments. For example, such an execution may correspond to a simulation of the logical operations as described herein. The software product may additionally or alternatively include number of instructions that enable a computer device to execute operations for configuring or programming a digital logic apparatus in accordance with example embodiments.

Example apparatuses and methods described herein, in accordance with example embodiments, can be implemented by one or more controllers. The controllers can comprise hardware, software, or a combination of hardware and software, depending on the particular application, component or function. In some example embodiments, the one or more controllers can include analog or digital components, and can include one or more processors, one or more non-transitory storage mediums such as memory storing instructions executable by the one or more processors, one or more transceivers (or separate transmitters and receivers), one or more signal processors (analog and/or digital), and/or one or more analog circuit components.

In the described methods or block diagrams, the boxes may represent events, steps, functions, processes, modules, messages, and/or state-based operations, etc. Although some of the above examples have been described as occurring in a particular order, it will be appreciated by persons skilled in the art that some of the steps or processes may be performed in a different order provided that the result of the changed order of any given step will not prevent or impair the occurrence of subsequent steps. Furthermore, some of the messages or steps described above may be removed or combined in other embodiments, and some of the messages or steps described above may be separated into a number of sub-messages or sub-steps in other embodiments. Even further, some or all of the steps may be repeated, as necessary. Elements described as methods or steps similarly apply to systems or subcomponents, and vice-versa. Reference to such words as “sending” or “receiving” could be interchanged depending on the perspective of the particular device.

The above discussed embodiments are considered to be illustrative and not restrictive. Example embodiments described as methods would similarly apply to systems, and vice-versa.

Variations may be made to some example embodiments, which may include combinations and sub-combinations of any of the above. The example embodiments presented above are merely examples and are in no way meant to limit the scope of this disclosure. Variations of the innovations described herein will be apparent to persons of ordinary skill in the art, such variations being within the intended scope of the present disclosure. In particular, features from one or more of the above-described embodiments may be selected to create alternative embodiments comprised of a sub-combination of features which may not be explicitly described above. In addition, features from one or more of the above-described embodiments may be selected and combined to create alternative embodiments comprised of a combination of features which may not be explicitly described above. Features suitable for such combinations and sub-combinations would be readily apparent to persons skilled in the art upon review of the present disclosure as a whole. The subject matter described herein intends to cover and embrace all suitable changes in technology.

The specification and drawings are, accordingly, to be regarded simply as an illustration, and are contemplated to cover any and all modifications, variations, combinations or equivalents. Certain adaptations and modifications of the described embodiments can be made. Therefore, the above discussed embodiments are considered to be illustrative and not restrictive.

1. A liquid fragrance dispensing system, comprising:
a liquid fragrance dispenser, comprising:
an atomizer that is configured to atomize a liquid fragrance in fluid communication with the atomizer and output the atomized liquid fragrance;
a dispenser memory; and
a dispenser controller for controlling operation of the atomizer, and for executing instructions stored in the dispenser memory that, when executed, causes the dispenser controller to detect a value of an external variable of an environment of the liquid fragrance dispensing system, the external variable having a plurality of possible values, and in response, variably control an output of the liquid fragrance dispenser.

2. The system of claim 1, comprising a sensor subsystem that is configured to sense the external variable and provide data in relation to the sensed external variable, and the dispenser controller is configured to receive the data from the sensor subsystem for the detecting of the value of the external variable.

3. The system of claim 2, wherein the output of the liquid fragrance dispenser is the atomization output of the atomizer, wherein the dispenser controller is configured to:
determine a target environmental fragrance condition information;
determine an actual environmental condition based on the amount of atomization output of the atomizer and the value of the external variable detected by the sensor subsystem;
wherein the variably control includes the dispenser controller being configured to variably control the atomization output of the atomizer to achieve the target environmental fragrance condition based on the actual environmental condition.

4. The system of claim 3, wherein:
the sensor subsystem includes a temperature sensor;
the actual environmental condition includes temperature; and
wherein the target environmental fragrance condition is achieved further based on the temperature.

5. The system of claim 3, wherein:
the sensor subsystem includes a humidity sensor;
the actual environmental condition includes humidity; and
wherein the target environmental fragrance condition is achieved further based on the humidity.

6. The system of claim 1, further comprising an accelerometer, and the dispenser controller is configured to:
determine acceleration threshold information;
receive, from the accelerometer, data representative of the acceleration of the housing detected by the accelerometer;
perform a comparison of the acceleration threshold information and the acceleration of the liquid fragrance dispenser; and
deactivate or not activate the atomizer when the acceleration of the liquid fragrance dispenser exceeds the acceleration threshold information.

7. The system of claim 3, wherein the target environmental fragrance condition is achieved further based on previous atomization output of the atomizer.

8. The system of claim 2, wherein the liquid fragrance dispenser further comprises a user interface subsystem having a lighting subsystem, wherein the output of the liquid fragrance dispenser includes light output from the lighting subsystem, wherein the sensor subsystem includes an occupancy detection sensor, and wherein the dispenser controller is configured to:
receive, from the occupancy detection sensor, data representative of an object's proximity detected by the occupancy detection sensor; and
wherein the variably control includes the dispenser controller being configured to variable activate the light output of the lighting subsystem based on the object's proximity.

9. The system of claim 2, wherein the output of the liquid fragrance dispenser is the atomization output of the atomizer, wherein the dispenser controller is configured to:
receive a control command in response to a determination of a proximity of a user equipment and the liquid fragrance dispenser based on GPS coordinates of the user equipment and GPS coordinates of the liquid fragrance dispenser; and
wherein the variably control includes the dispenser controller being configured to variably control the atomization output of the atomizer based on the proximity.

10. The system of claim 2, wherein the liquid fragrance dispenser further comprises a user interface subsystem having a lighting subsystem, wherein the output of the liquid fragrance dispenser includes lighting intensity from the lighting subsystem, wherein the sensor subsystem includes an ambient light sensor, and the controller is configured to:
receive, from the ambient light sensor, data representative of an intensity of the ambient light detected by the ambient light sensor; and
wherein the variably control includes the dispenser controller being configured to variably control the lighting intensity of the lighting subsystem in general proportion to the intensity of the ambient light.

11. The system of claim 1, wherein said variably controlling is based on a characteristic of the liquid fragrance.

12. The system of claim 11, wherein the characteristic includes at least one of: a fluid type of the liquid fragrance, an odor intensity scale of the liquid fragrance, a fragrance decay rate of the liquid fragrance, a concentration of a solute in the liquid fragrance, or a viscosity of the liquid fragrance.

13. The system of claim 2, wherein the liquid fragrance dispenser further comprises a wireless communication subsystem, the system further comprising:
a liquid fragrance cartridge that is releasably couplable to the atomizer, the liquid fragrance cartridge comprising:
a container that defines a cavity to contain the liquid fragrance;
a port for providing fluid communication to the cavity of the container;
a cartridge memory for storing information representative of the liquid fragrance cartridge;
a wireless communication subsystem; and
a cartridge controller for executing instructions stored in the cartridge memory that, when executed, causes the cartridge controller to wirelessly transmit data representative of the liquid fragrance cartridge to the liquid fragrance dispenser;
wherein the dispenser controller is configured to:
receive the data representative of the liquid fragrance cartridge transmitted by the cartridge controller.

14. The system of claim 13, wherein the variably control is based on the data representative of the liquid fragrance cartridge.

15. The system of claim 13, wherein the data representative of the liquid fragrance cartridge received by the dispenser controller from the cartridge controller includes is a unique identification of the liquid fragrance cartridge, wherein the dispenser controller is configured to:
validate the unique identification of the liquid fragrance cartridge from the dispenser memory or through communication with an external device.

16.-27. (canceled)

28. A liquid fragrance cartridge, comprising:
a container that defines a cavity to contain a liquid fragrance;
a port for providing fluid communication to the cavity of the container;
a memory for storing information representative of the liquid fragrance cartridge;
a wireless communication subsystem; and
a controller for executing instructions stored in the memory that, when executed, causes the controller to wirelessly transmit data representative of the liquid fragrance cartridge.

29.-34. (canceled)

35. The cartridge of claim 0, wherein the memory stores information representative of a unique identification of the liquid fragrance cartridge, and wherein the transmit data includes transmitting data representative of the unique identification.

36. The cartridge of claim 0, wherein the controller is configured to receive data representative of confirmation of validity of the unique identification of the liquid fragrance cartridge.

37. The system of claim 0, wherein the controller is configured to perform the transmitting of the data representative of the unique identification of the liquid fragrance cartridge using a short range communication protocol.

38.-43. (canceled)

44. A non-transitory computer-readable medium containing instructions executable by a controller of a liquid fragrance dispenser, the liquid fragrance dispenser having an atomizer that is configured to atomize a liquid fragrance in fluid communication with the atomizer and output the atomized liquid fragrance, the method comprising:
instructions for detecting a value of an external variable of an environment of the liquid fragrance dispensing system, the external variable having a plurality of possible values; and
instructions for, in response to the detecting, variably control an output of the liquid fragrance dispenser.
